(19) United States
(12) Patent Application Publication
(10) Pub. No.: US 2023/0285188 A1
(43) Pub. Date: Sep. 14, 2023

(54) GLAUCOMA TREATMENT VIA INTRACAMERAL OCULAR IMPLANTS

(71) Applicant: Allergan, Inc., Irvine, CA (US)

(72) Inventors: Tomas Navratil, Morrisville, NC (US); Sanjib Das, Morrisville, NC (US); Andres Garcia, Morrisville, NC (US); Janet Tully, Morrisville, NC (US)

(21) Appl. No.: 17/819,284

(22) Filed: Aug. 11, 2022

Related U.S. Application Data
(63) Continuation of application No. 17/456,314, filed on Nov. 23, 2021, now abandoned.
Application No. 17/456,314 is a continuation of application No. 16/637,676, filed on Feb. 7, 2020, now abandoned.
(60) Provisional application No. 62/352,408, filed on Jun. 20, 2016.
(60) Provisional application No. 62/329,736, filed on Apr. 29, 2016.
(60) Provisional application No. 62/321,581, filed on Apr. 12, 2016.
(60) Provisional application No. 62/277,251, filed on Jan. 11, 2016.
(60) Provisional application No. 62/237,443, filed on Oct. 5, 2015.
(60) Provisional application No. 62/196,209, filed on Jul. 23, 2015.

(51) Int. Cl. A61K 9/00; A61F 9/007; A61K 31/5575

(52) U.S. Cl. CPC A61K 9/0051; A61F 9/00781; A61K 31/5575; A61P 27/02

(57) ABSTRACT

The disclosure teaches methods of utilizing precisely engineered biodegradable drug delivery systems to treat intraocular conditions. In aspects, the disclosure provides methods of treating elevated intraocular pressure with intracameral implants administered to the anterior region of an eye. Furthermore, the disclosure provides for methods of lowering intraocular pressure in a subject, by administering intracameral implants that maintain a multi-month sustained level of travoprost acid in the aqueous humor of said subject’ eye, which is at least 8× lower than the EC50 values of travoprost acid on its molecular target, but yet still achieves clinically significant lowering of IOP.

### CROSS-REFERENCE TO RELATED APPLICATIONS

This application is continuation of U.S. Pat. Application Serial No. 16/637,676, filed Feb. 7, 2020, which is a national stage application under 371 U.S.C. § 371 of PCT Application PCT/US2016/043951, filed Jul. 25, 2016 which claims priority to: U.S. Provisional Application No. 62/196,209, filed Jul. 23, 2015; U.S. Provisional Application No. 62/237,443, filed Oct. 5, 2015; U.S. Provisional Application No. 62/277,251, filed Jan. 11, 2016; U.S. Provisional Application No. 62/321,581, filed Apr. 12, 2016; U.S. Provisional Application No. 62/329,736, filed Apr. 29, 2016; and U.S. Provisional Application No. 62/352,408, filed Jun. 20, 2016, which are incorporated by reference herein in their entireties and serve as the basis of a priority and/or benefit claim for the present application.

### FIELD

The present disclosure relates to the field of treating ocular conditions via the utilization of ocular implant delivery vehicles to administer pharmaceutical agents to targeted anatomical regions of the eye.

### BACKGROUND

Glaucoma is a progressive optic neuropathy affecting more than three million Americans over the age of 39 and is a leading cause of blindness in adults over age 60. According to the National Eye Institute, more than 120,000 Americans are blind due to glaucoma (Quigley HA, Vitale S. “Models of open-angle glaucoma prevalence and incidence in the United States,” Invest Ophthalmol & Visual Sci. 1997, 38(1):83-91.).

Elevated intraocular pressure (IOP) is the most important risk factor for the development of glaucoma and is a result of abnormally high resistance to aqueous humor drainage through the trabecular meshwork (TM), a multi-laminar array of collagen beams covered by endothelial-like cells.

Due to limited understanding of the pathophysiology of the optic neuropathy characteristic of glaucoma, current glaucoma therapies are focused on reducing IOP. The prostaglandin analogues (PGAs) are currently the most prescribed class of topical therapies for ocular hypertension or glaucoma in the United States. However, their use has been limited by several shortcomings.

First, the compliance with existing glaucoma topical therapies is generally low, with 30% to 60% of patients discontinuing the therapy within the first year of treatment.

Second, topical ophthalmic agents currently in use have local and systemic side effects. For example, these agents have a relatively high incidence of hyperemia accompanied by drug level peaks and troughs in the aqueous humor and the surrounding tissues, which potentially leads to 24 hour IOP fluctuations that may contribute to accelerated loss of visual field in susceptible patients (Caprioli J, Roht V. “Intraocular Pressure: Modulation as treatment for Glaucoma,” Am J Ophthalmol. 2011;152(3):340-344.)

Third, topical administration of currently approved formulations of PGAs, such as TRAVATAN Z®, to the from of the eye is not efficacious and results in only a small fraction of the total dose reaching the site of action due to low efficiency of transport through the cornea.

Lastly, the combination of these factors has been shown to increase the cost of patient care due to faster disease progression.

Therefore, there is a great need in the medical field for an alternative treatment using a sustained-release delivery system with an improved safety and efficacy profile. To date, there are no United States Food and Drug Administration (FDA) approved glaucoma therapies providing sustained release of a pharmacological agent directly to the desired site of action. Therefore, a sustained release pharmaceutical formulation administered directly to the anterior chamber of an eye would likely improve both compliance and the adverse event profile of current IOP-lowering drugs. Moreover, any extended release implant is highly dependent on the selection of polymers, co-polymers, drug-polymer interaction, load uniformity, porosity, size, surface-area to volume ratio, and the like for providing its drug release and degradation characteristics and the manufacturing techniques used in the prior art implants can induce inherent drawbacks in each of these parameters.

### BRIEF SUMMARY

The present disclosure addresses a crucial need in the art, by providing a sustained-release pharmaceutical formulation that may be directly administered to the anterior chamber of an eye and that does not suffer from the drawbacks of the current art.

Moreover, the present disclosure provides ocular implants with highly uniform, tunable and reproducible size, shape, loading, composition, and load distribution, which provide implants having a desired extended drug release profile suitable for treating desired indications. In a particular embodiment, the implant is utilized to treat an ocular indication of an increased ocular pressure.

The biodegradable drug delivery systems taught herein arc, in some embodiments, engineered using a Particle Replication in Non-wetting Template (PRINT®) technology. The PRlNT® Technology utilized in some embodiments allows for uniform size, shape, and dose concentration in the disclosed drug delivery systems,

In some embodiments, the ocular implants comprise at least one therapeutic agent selected from the group consisting of a prostaglandin, prostaglandin prodrug, prostaglandin analogue, and prostamide, pharmaceutically acceptable salts thereof, and mixtures thereof. In particular embodiments, the therapeutic agent is selected from the group consisting of latanoprost, travoprost, bimatoprost, tafluprost, and unoprostone isopropyl. In one embodiment, the at least one therapeutic agent comprises travoprost.

Further, the disclosure provides methods of utilizing the taught precisely engineered biodegradable drug delivery systems to treat, inter alia, conditions of the eye.

Conditions treatable according to the present disclosure include glaucoma, elevated intraocular pressure, and ocular hypertension.

In one aspect, the disclosure provides for newly identified, significantly lower levels of PGA in aqueous humor sufficient for IOP lowering when achieved via sustained release of PGA. That is, the inventors have surprisingly discovered that when administering a prostaglandin analog (PGA), e.g. travoprost, directly into the anterior chamber of human subjects in a sustained release manner, the levels of PGAs in the aqueous humor needed to lower lOP in human subjects are significantly lower than PGA levels previously considered as necessary for lOP-lowering effect in humans.

In some embodiments, the level of PGA in the aqueous humor achieved using the present implants is from about 0.001 nMol/L to about 2 nMol/L, from about 0.01 nMol/L to about 1.4 nMol/L, from about 0.01 nMol/L to about 1.3 nMol/L, from about 0.01 nMol/L to about 1.2 nMol/L, from about 0.01 nMol/L to about 1.1 nMol/L, from about 0.01 nMol/L to about 1.0 nMol/L, from about 0.01 nMol/L to about 0.9 nMol/L, from about 0.01 nMol/L to about 0.8 nMol/L, from about 0.01 nMol/L to about 0.7 nMol/L, from about 0.01 nMol/L to about 0.6 nMol/L, from about 0.01 nMol/L to about 0.5 nMol/L, from about 0.01 nMol/L to about 0.5 nMol/L, from about 0.01 nMol/L to about 0.4 nMol/L, from about 0.01 nMol/L to about 0.3 nMol/L, from about 0.01 nMol/L to about 0.2 nMol/L, from about 0.01 nMol/L to about 0.1 nMol/L, from about 0.01 nMol/L to about 0.09 nMol/L, from about 0.01 nMol/L to about 0.8 nMol/L, from about 0.01 nMol/L to about 0.7 nMol/L from about 0.01 nMol/L to about 0.06 nMol/L from about 0.01 nMol/L to about 0.05 nMol/L, from about 0.01 nMol/L to about 0.04 nMol/L, from about 0.01 nMol/L to about 0.03 nMol/L, including all values and subranges in between. In particular embodiments, the level of PGA in the aqueous humor is less than or equal to about 0.051 nMmol/L. In some aspects, the level of PGA in the aqueous humor is from about 0.0327 to about 0.1793 nMol/L In particular embodiments, the level of PGA in the aqueous humor is less than or equal to about 0.165 nMol/L. In other aspects, the level of PGA in the aqueous humor is from about 0.0766 to about 0.3795 nMol/L.

In some embodiments, the level of PGA in the aqueous humor is from about 0.03 nMol/L to about 1.4 nMol/L, from about 0.03 nMol/L to about 1.3 nMol/L. from about 0.03 nMol/L to about 1.2 nMol/L, from about 0.03 nMol/L to about 1.1 nMol/L, from about 0.03 nMol/L to about 1.0 nMol/L, from about 0.03 nMol/L to about 0.9 nMol/L, from about 0.03 nMol/L to about 0.8 nMol/L, from about 0.03 nMol/L to about 0.7 nMol/L, from about 0.03 nMol/L to about 0.6 nMol/L, from about 0.03 nMol/L to about 0.5 nMol/L, from about 0.03 nMol/L to about 0.5 nMol/L, from about 0.03 nMol/L to about 0.4 nMol/L, from about 0.03 nMol/L to about 0.3 nMol/L, from about 0.03 nMol/L to about 0.2 nMol/L, from about 0.03 nMol/L to about 0.1 nMol/L, from about 0.03 nMol/L to about 0.09 nMol/L, from about 0.03 nMol/L to about 0.08 nMol/L, from about 0.03 nMol/L to about 0.07 nMol/L, from about 0.03 nMol/L to about 0.06 nMol/L, from about 0.03 nMol/L to about 0.05 nMol/L, including all values and subranges in between.

In some embodiments, the level of PGA in the aqueous humor is from about 0.05 nMol/L to about 0.2 nMol/L, from about 0.05 nMol/L to about 0.19 nMol/L, from about 0.05 nMol/L to about 0.18 nMol/L, from about 0.05 nMol/L to about 0.17 nMol/L, from about 0.05 nMol/L to about 0.16 nMol/L, from about 0.05 nMol/L to about 0.15 nMol/L, from about 0.05 nMol/L to about 0.14 nMol/L, from about 0.05 nMol/L to about 0.13 nMol/L, from about 0.05 nMol/L to about 0.12 nMol/L, from about 0.05 nMol/L to about 0.11 nMol/L, from about 0.05 nMol/L to about 0.10 nMol/L, or about 0.06 nMol/L to about 0.2 nMol/L, from about 0.06 nMol/L to about 0.19 nMol/L. from about 0.06 nMol/L to about 0.18 nMol/L, from about 0.06 nMol/L to about 0.17 nMol/L, from about 0.06 nMol/L to about 0.16 nMol/L, from about 0.06 nMol/L to about 0.15 nMol/L, from about 0.06 nMol/L to about 0.14 nMol/L, from about 0.06 nMol/L to about 0.13 nMol/L, from about 0.06 nMol/L to about 0.12 nMol/L, from about 0.06 nMol/L to about 0.11 nMol/L, from about 0.06 nMol/L to about 0.10 nMol/L from, including all values and subranges in between. In certain embodiments, the level of PGA in the aqueous humor is from about 0.0327 nMol/L to about 0.380 nMol/L. In certain embodiments, the level of PGA in the aqueous humor is from about 0.0327 nMol/L to about 0.1793 nMol/L. In certain embodiments, the level of PGA in the aqueous humor is from about 0.0766 nMol/L to about 0.380 nMol/L, In certain embodiments, the level of PGA in the aqueous humor is in the range of about 0.051 nMmol/L to about 0.165 nMol/L.

In embodiments. IOP is reduced below a baseline by about 1% to about 100%, or about 10% to about 90%, or about 10% to about 80%, or about 10% to about 70%, or about 10% to about 60%, or about 10% to about 50%, or about 10% to about 50%, or about 10% to about 30%, or about 20% o to about 90%or about 20% to about 80%, or about 20% to about 70%, or about 20% to about 60%, or about 20% to about 50%, or about 20% o to about 40%, or about 20% to about 30%.

In embodiments. IOP is reduced by an amount in the range of about 1 mmHg to about 15 mmHg, or about 3 mmHg to about 15 mmHg, or about 5 mmHg to about 15 mmHg. In embodiments, IOP is reduced below about 25 mmHg, or about 24 mmHg, or about 23 mmHg, or about 22 mmHg, or about 21 mmHg, or about 20 mmHg, or about 19 mmHg, or about 18 mmHg, or about 17 mmHg, or about 16 mmHg, or about 15 mmHg, or about 14 mmHg, or about 13 mmHg, or about 12 mmHg, or about 11 mmHg, or about 10 mmHg

Additionally and surprisingly, the levels of PGA sufficient for IOP lowering are also far below the EC₅₀ levels of these PGAs on their molecular target, the FP receptor (see FIG. 5 for IOP lowering effects in human subjects). In some embodiments, the level of PGA is reduced below the EC₅₀ by about 1% to about 100%, or about 10% to about 99%, or about 15% to about 99%, or about 20% to about 99%o, or about 25% to about 99%, or about 30% to about 99%, or about 35% to about 99%, or about 40% to about 99%, or about 45% to about 99%, to about 50% to about 99%, or about 55% or about 99%, or about 60% to about 99%, or about 65% to about 99%, or about 70% to about 99%, or about 750% to about 99%, or about 80% to about 99%, or about 85% to about 99%, or about 90% to about 99%, or about 95% to about 99%, including all values and subranges in between. In some embodiments, the level of PGA is reduced below the EC₅₀ by at least about 99%, at least about 95%, at least about 90%, at least about 85%, at least about 80%, at least about 70%, about 60%, at least about 50%, at least about 40%, at least about 30%, at least about 20%, or at least about 10%.

In some embodiments, lOP-lowering was demonstrated in human subjects at PGA levels in aqueous humor of from about 2X to about 50X, or about 2X, about 3X, about 4X, about 5X, about 6X, about 7X, about 8X, about 9X, about 10X, about 11X, about 12X, about 13X, about 14X, about 15X, about 16X, about 17X, about 18X, about 19X, about 20X, about 21X, about 22X, about 23X, about 24X, about 25X, about 26X, about 27X, about 28X, about 29X, or about 30X below the EC₅₀ values of PGA on its molecular target, the FP receptor

In embodiments, the implants disclosed herein can be formulated to provide a non-linear release of a therapeutic agent (e.g., initial burst and subsequent fluctuations in the release of the therapeutic agent). Surprisingly, clinically significant lowering of IOP was maintained (e.g., at least about 7 months) with implants formulated to exhibit a non-linear release of a prostaglandin analog. In some embodiments, the implants may be formulated to release the therapeutic agent below the EC₅₀ of the therapeutic agent on its molecular target and, surprisingly, achieve clinically significant lowering of IOP for at least about 7 months. In embodiments, the prostaglandin analog concentration in the aqueous humor can fluctuate by about ± 5%, ± 10%, ± 15%, ± 20%, ± 25%, ± 30%, ± 35%, ± 40%, ± 45%, or ± 50% while maintaining levels sufficient for clinically significant lowering of IOP. For example, in some embodiments in which the PGA is travoprost, the concentration of travoprost acid in the aqueous humor is 0.051 nMol/L and fluctuates by about ± 50% (e.g., ±40%, ±30%, ± 25%, ± 20%, ± 15%, ± 10%, or ± 5%). In other embodiments, the concentration of travoprost acid in the aqueous humor is 0.165 nMol/L and fluctuates by about ± 50% (e g., ±40%, ±30%, ±25%, ± 20%, ± 15%, ± 10%, or ± 5%).

In other embodiments, the implants disclosed herein can be formulated to provide a linear release of a therapeutic agent. In such embodiments, clinically significant lowering of IOP is maintained (e.g., at least about 7 months) with implants formulated to exhibit a linear release of a therapeutic agent. In some embodiments, the implants may be formulated to release the therapeutic agent below the EC₅₀ of the therapeutic agent on its molecular target and, surprisingly, achieve clinically significant lowering of IOP for at least about 7 months. In some embodiments in which the PGA is travoprost, the concentration of travoprost acid in the aqueous humor is about 0.051 nMol/L ± 50% In other embodiments, the concentration of travoprost acid in the aqueous humor is about 0.165 nMol/L ± 50%.

In certain embodiments, the methods provide for IOP lowering effects for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 2 years, at least about 3 years, at least about 4 years, or at least about 5 years

In embodiments, clinically significant IOP lowering is achieved within 15 days after administration of an implant, e.g., within 14 days, within 13 days, within 12 days, within 11 days, within 10 days, within 9 days, within 8 days, within 7 days, within 6 days, within 5 days, within 4 days, within 3 days, within 2 days, or within 1 day

In certain embodiments, the intracameral implants are designed to provide PGA levels which are below the EC₅₀ levels of these PGAs on their molecular target, the FP receptor. In certain embodiments, PGA levels in the aqueous humor may fluctuate by about ± 5%, about ± 10%, about ± 15%, about ± 20%, about ± 25%, or about ± 30%. That is, PGA levels in the aqueous humor may fluctuate (e.g., by as much as ± 30%) while maintaining reduced IOP. For example, in some embodiments, a PGA (e.g., travoprost acid) concentration in the aqueous humor of about 0.051 nMol/L ±50% is maintained for at least 7 months In embodiments, the PGA (e.g, travoprost acid) concentration in the aqueous humor fluctuates within ±5%, ±10%, ±15%, ±20%, ± 25%, ±30%, ±40%, or ±50% of 0.051 nMol/L. Thus, in certain embodiment, the PGA (e.g., travoprost acid) concentration in the aqueous humor is in the range of about 0.0327 to about 0.179 nMol/L. In embodiments, a PGA (e g., travoprost acid) concentration in the aqueous humor of about 0.165 nMol/L ±50% is maintained for at least 7 months. In embodiments, the PGA (e.g, travoprost) concentration in the aqueous humor fluctuates within ±5%, ±10%, ±15%, ±20%,± 25%, ±30%, ±40%, or ±50% of 0.165 nMol/L. Thus, in certain embodiment, the PGA (e.g., travoprost acid) concentration in the aqueous humor is in the range of about 0.0766 to about 0.380 nMol/L.

Thus, in one embodiment, a robust IOP-lowering was demonstrated in human subjects at travoprost acid levels in aqueous humor 8 to 28 x lower than the EC₅₀ values of travoprost acid on its molecular target, the FP receptor. Based on this finding, the inventors identified new target levels of PGAs in the aqueous humor that are particularly useful to treatment of ocular hypertension in glaucoma patients, when achieved via sustained release formulations of PGAs, and that were previously considered sub-therapeutic and not eliciting the desired IOP-lowering treatment effect

Furthermore, the inventors similarly identified new target levels in the aqueous humor for other IOP-lowering agents that are particularly useful for treatment of ocular hypertension in glaucoma patients, when achieved via sustained release formulations of these agents, and that were previously considered sub-therapeutic and not eliciting the desired lOP-lowering treatment effect. The new target levels in the aqueous humor when achieved via sustained release formulations were identified for these agents beta-blockers such as timolol, alpha-adrenergic agents such as brimonidine, carbonic anhydrase inhibitors such as brinzolamide, EP receptor agonists, rho kinase inhibitors, PGAs with no donating groups, and others.

These findings enable IOP lowering and consequent prevention or slowdown of progressive vision loss in glaucoma patients at these newly identified, significantly lower levels of PGA in aqueous humor sufficient for IOP lowering when achieved via sustained release formulations, with significant advantages and benefits for the patients suffering from glaucoma.

These newly identified, significantly lower levels of PGA in aqueous humor sufficient for IOP lowering when achieved via sustained release formulations have the following advantages: I) they are dramatically dose sparing in that they enable discovery and development of new therapeutic products that contain significantly lower dose of PGA compared to other PGA formulations including topical ophthalmic formulations of PGAs needed for IOP-lowering treatment over the same time period; 2) they lead to lower tissue exposures and thus offer potential for improved safety over existing approaches; 3) they enable discovery and development of new IOP-lowering sustained release PGA products that lower IOP over longer period of time compared to previous PGA products containing the same dose of PGA; 4) they enable the discovery and development of new IOP-lowering sustained release PGA products that are smaller in size and thus can be administered via a smaller needle when injected or into a smaller anatomical space inside the eye or in the vicinity of the eye; and 5) they decrease the potential for tachyphylaxis or loss of treatment effect occurring due to, but not limited to, receptor internalization, degradation, and decrease of the overall receptor copy number when PGA receptors and other molecular targets are exposed to the PGAs.

Thus, one embodiment of the disclosure provides for a method for lowering intraocular pressure in a human subject in need thereof, comprising: a) administering at least one intracameral implant to the anterior chamber of said subject’s eye, wherein said intracameral implant comprises a biodegradable polymer matrix and at least one prostaglandin analog homogeneously dispersed therein, and wherein said intracameral implant achieves a prostaglandin analog concentration in the aqueous humor of about 0.051 nMol/L to about 0.165 nMol/L, and wherein the intraocular pressure in said subject’s eye is lowered. In certain aspects, the prostaglandin analog is travoprost and travoprost acid is maintained at the aforementioned levels in the aqueous humor.

In another embodiment, the disclosure provides for a method for lowering intraocular pressure in a subject’s eye, comprising: a) administering travoprost to the anterior chamber of said subject’s eye, such that a level of travoprost acid is achieved in the aqueous humor of said subject’ eye, which is at least 8x lower than the EC₅₀ value of travoprost acid on its molecular target, and wherein clinically significant lowering of IOP is sustained. In some aspects, the travoprost is administered via an intracameral implant.

In another embodiments, the disclosure provides for reducing

In another embodiment, the disclosure provides for a method for lowering intraocular pressure in a subject’s eye, comprising: a) administering travoprost to the anterior chamber of said subject’s eye, such that a level of travoprost acid is achieved in the aqueous humor of said subject’ eye, which is at least 28x lower than the EC₅₀ value of travoprost acid on its molecular target, and wherein clinically significant lowering of IOP is sustained. In some aspects, the travoprost is administered via an intracameral implant Accordingly, in embodiments disclosed herein, the method for lowering intraocular pressure comprises: administering at least one intracameral implant to the anterior chamber of said subject’s eye, wherein said intracameral implant comprises a biodegradable polymer matrix and at least one therapeutic agent homogenously dispersed therein. In certain embodiments, the biodegradable polymer matrix comprises as a % w/w of the overall intracameral implant composition about 5% to about 95% w/w, or about 5%to about 90% w/w, or about 5% to about 80%, or about 5% to about 70%, or about 5% to about 60%, or about 10% to about 90% w/w, or about 10% to about 80%, or about 10% to about 70%, or about 10% to about 60%, or about 20% to about 90%, or about 20% to about 80%, or about 20% to about 70%, or about 20% to about 60%, or about 30% to about 90%, or about 30% to about 80%, or about 30% to about 70%, or about 30% to about 60%, or about 40% to about 90%, or about 40% to about 80%, or about 40% to about 70%, or about 40% to about 60%, or about 50% to about 90%, or about 50% to about 80%, or about 50% to about 70%, or about 50% to about 60%, or about 60% to about 90%, or about 60% to about 85%, or about 65% to about 85%, or about 60% to about 80%, or about 60% to about 70%; or about 45% to about 80%, or about 45% to about 75%, or about 45% to about 70%, or about 45% to about 65%, or about 45% to about 60%, or about 45% to about 55%, or about 45% to about 50%, or about 70% to about 80%, or about 65% to about 85%, or about 85% to about 95%, or about 92.5% to about 95%, or about 55% to about 70% w/w of the intracameral implant composition.

In certain embodiments, the biodegradable polymer matrix comprises as a % w/w of the intracameral implant: about 10% to about 90% w/w, or about 10% to about 80%, or about 10% to about 70%, or about 10% to about 60%, or about 20% to about 90%, or about 20% to about 80%, or about 20% to about 70%, or about 20% to about 60%, or about 30% to about 90%, or about 30% to about 80%, or about 30% o to about 70%, or about 30% to about 60%, or about 40% to about 90%, or about 40% to about 80%, or about 40% to about 70%, or about 40% to about 60%, or about 50% to about 90%, or about 50% to about 80%, or about 50% to about 70%, or about 50% to about 60%, or about 60% to about 90%, or about 60% to about 80%, or about 60% to about 75%, or about 60% to about 70%, or about 65% to about 75%, or about 68% to about 71%, or about 70%, or about 50 % to about 70%, or about 55% to about 65%, or about 55% to about 61%, w/w of the pharmaceutical composition.

In certain embodiments, the biodegradable polymer matrix includes a first polymer. In aspects, the first polymer comprises as a % w/w of the biodegradable polymer matrix: about 1% to about 100%, or about 1% to about 90% w/w, or about 1% to about 80%, or about 1% to about 70%, or about 1% to about 60%, or about 1% to about 50%, or about 1% to about 40%, or about 1% to about 30%, or about 1% to about 20%, or about 1% to about 15%, or about 1% to about 10%, or about 1% to about 5%, or about 20% to about 90%, or about 25% to about 80%, or about 30% to about 70%, or about 20% to about 40%, or about 25% to about 35%, including all values and subranges in between In aspects, the first polymer comprises as a % w/w of the biodegradable polymer matrix: about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%. In aspects, the first polymer is a PLA polymer. In aspects, the PLA polymer is R 208 S. In aspects, the PLA polymer (e.g., R 203 S) can be present as the sole polymer in the biodegradable polymer matrix. In aspects, the PLA polymer (e.g., R 203 S) can be present in a mixture of polymers in the biodegradable polymer matrix.

In certain embodiments, the biodegradable polymer matrix includes a first polymer. In aspects, the first polymer comprises as weight of the biodegradable polymer matrix: about 1 µg to about 1,000 µg, about 1 µg to about 500 µg, or about 1 µg to about 400 µg, or about 1 µg to about 300 µg, or about 1 µg to about 200 µg, or about 1 µg to about 100 µg, or about 1 µg to about 90 µg, or about 1 µg to about 80 µg, or about 1 µg to about 70 µg, or about 1 ug to about 60 µg, or about 1 µg to about 50 µg, or about 1 µg to about 40 µg, or about 1 µg to about 30 µg, or about 1 µg to about 20 µg, or about 1 µg to about 10 µg, including all values and subranges in between. In aspects, the first polymer comprises as weight of the biodegradable polymer matrix: about 5 µg to about 70 µg, or about 5 µg to about 15 µg, or about 7 µg to about 12 ug, or about 8 to about 10 µg, or about 9 µg, or about 25 µg to about 35 µg, or about 26 µg to about 32 µg, or about 26 µg to about 30 µg, or about 28 µg. In aspects, the first polymer is a PLA polymer, including all values and subranges in between. In aspects, the PLA polymer is R 203 S. In aspects, the PLA polymer (e.g., R 203 S) can be present as the sole polymer in the biodegradable polymer matrix. In aspects, the PLA polymer (e.g., R 203 S) can be present in a mixture of polymers in the biodegradable polymer matrix.

In certain embodiments, the biodegradable polymer matrix includes a second polymer. In aspects, the second polymer comprises as a % w/w of the biodegradable polymer matrix; about 1% to about 100%, or about 1% to about 90% w/w, or about 1% to about 80%, or about 1% to about 70%, or about 1% to about 60%, or about 1% to about 50%, or about 1% to about 40%, or about 1% to about 30%, or about 1% to about 20%, or about 1% to about 15%, or about 1% to about 10%, or about 1% to about 5%, or about 20% to about 90%, or about 25% to about 80%, or about 30% to about 70%, or about 50% to about 90%, or about 60% to about 80%, or about 65% to about 75%. In aspects, the second polymer comprises as a % w/w of the biodegradable polymer matrix; about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%. In embodiments, the second polymer is a PLA polymer. In aspects, the PLA polymer is R 208. In embodiments, the PLA polymer (e.g., R 208) can be present in a mixture of polymers in the biodegradable polymer matrix. In aspects, the second polymer is a PLGA polymer In aspects, the PLGA polymer is RG 750S. In aspects, the PLGA polymer can be present in a mixture of polymers in the biocompatible polymer matrix, e.g., in a PLGA/PLA mixture.

In certain embodiments, the biodegradable polymer matrix includes a second polymer. In aspects, the second polymer comprises as weight of the biodegradable polymer matrix: about 1 µg to about 1,000 µg, about 1 µg to about 500 µg, or about 1 µg to about 400 µg, or about 1 ug to about 300 ug, or about 1 µg to about 200 µg, or about 1 µg to about 100 µg, or about 1 µg to about 50 µg, or about 1 µg to about 40 µg, or about 1 µg to about 30 µg, or about 1 µg to about 20 µg, or about 1 µg to about 10 µg, or about 1 to about 5 µg. In aspects, the second polymer comprises as weight of the biodegradable polymer matrix; about 10 µg to about 70 µg, or about 10 µg to about 30 µg, or about 12 µg to about 25 µg, or about 15 µg to about 20 µg, or about 18 µg to about 19 µg, or about 50 µg to about 75 µg, or about 55 µg to about 70 µg, or about 55 µg to about 65 µg, or about 55 µg to about 60 µg, or about 58 µg. In aspects, the second polymer is a PLA polymer. In aspects, the PLA polymer is R 208. In aspects, the PLA polymer (e.g., R 208) can be present as the sole polymer in the biodegradable polymer matrix. In aspects, the second polymer is a PLGA polymer. In aspects, the PLGA polymer is RG 705 S. In aspects, the PLGA polymer can be present in a mixture of polymers in the biocompatible polymer matrix, e.g., in a PLGA/PLA mixture.

In certain embodiments, the biodegradable polymer matrix includes a first polymer and a second polymer. In aspects, the first polymer and the second polymer comprise as a % w/w ratio of the biodegradable polymer matrix: about 1%/99% to about 99%/1%, or about 5%/95% to about 95%/5%, or about 10%/90% to about 90%/10%, or about 15%/85% to about 85%/15%, or about 20%/80% to about 80%/20%, or about 25%/75% to about 75%/25%, or about 30%/70% to about 70%/30%, or about 35%/65% to about 65%/35%, or about 40%/60% to about 60%/40%, or about 45%/55% to about 55%/45%, or about 50%/50% In embodiments, the first polymer and the second polymer comprises as a % w/w ratio of the biodegradable polymer matrix: about 30%/70% or about 33%/67%. In embodiments, biodegradable polymer matrix contains a mixture of polymers comprising as a wt% per implant i) 22 +/- 5% of a biodegradable poly(D.L-lactide) homopolymer having an inherent viscosity of 0.25 to 0.35 dL/g measured at 0.1% w/v in CHCl₃ at 25° C. with a Ubbelhode size 0c glass capillary viscometer; and ii) 45 +/-5% of a biodegradable poly(D,L-lactide) homopolymer having an inherent viscosity of 1.8 to 2.2 dL/g measured at 0.1% w/v in CHCl₃ at 25° C. with a Ubbelhode size 0c glass capillary viscometer. In embodiments, the first polymer and the second polymer are PLA polymers. In embodiments, the first polymer is a R 203 S polymer, and the second polymer is a R 208 polymer

In certain embodiments, the biodegradable polymer matrix includes a first polymer and a second polymer. In aspects, the first polymer and the second polymer comprise as a % w/w ratio of the biodegradable polymer matrix: about 1%/99% to about 99%/1%, or about 5%/95% to about 95%/5%, or about 10%/90% to about 90%/10%, or about 15%/85% to about 85%/15%, or about 20%/80% to about 80%/20%, or about 25%/75% to about 75%/25%, or about 30%/70% to about 70%/30%, or about 35%/65% to about 65%/35%, or about 40%/60% to about 60%/40%, or about 45%/55% to about 55%/45%, or about 50%/50%. In embodiments, the first polymer and the second polymer comprises as a % w/w ratio of the biodegradable polymer matrix: about 10%/90% or about 20%/80%. In embodiments, the biodegradable polymer matrix contains a mixture of polymers comprising as a wt % per implant: i) 9 +/- 5% of ester end-capped biodegradable poly(D,L-lactide-coglycolide) copolymer having an inherent viscosity of 0.8 to 1.2 dL/g measured at 0.1% w/v in CHCl₃ at 25° C. with a Ubbelhode size 0c glass capillary viscometer; and ii) 49 +/- 5% of ester end-capped biodegradable poly(D,L-lactide) homopolymer having an inherent viscosity of 1.8 to 2.2 dL/g measured at 0.1% w/v in CHCl₃ at 25° C. with a Ubbelhode size 0c glass capillary viscometer. In embodiments, the first polymer is a PLGA polymer and the second polymer is a PLA polymer. In embodiments, the first polymer is a RG 750 S polymer, and the second polymer is a R 208 polymer.

In certain embodiments, the biodegradable polymer matrix is comprised of a first polymer and a second polymer In aspects, the first polymer and the second polymer respectively comprises as a weight of the biodegradable polymer matrix: about 1 µg to about 1000 µg and about 1 µg to about 1000 µg; or about 1 µg to about 100 µg and about 500 µg to 1000 µg; or about 3 µg to about 50 µg and about 10 µg to 100 µg; or 3 µg to about 30 µg and about 10 µg to 50 µg, or about 5 µg to 15 µg and about 15 µg to about 25 µg; or about 7 µg to about 12 µg and about 16 µg to about 20 µg, or about 10 µg to about 50 µg and about 25 µg to about 100 µg, or about 15 µg to about 40 µg and about 30 µg to about 75 µg; or about 20 µg to about 35 µg and about 40 µg to about 65 µg; or about 25 µg to about 30 µg and about 50 µg to about 65 µg; or about 9 ug and about 18 µg; or about 28 µg and about 57 µg; or about 2 µg to about 7 µg and about 30 µg to about 40 µg.

In certain embodiments, the biodegradable polymer matrix includes a third polymer. In aspects, the third polymer comprises as a % w/w of the biodegradable polymer matrix: about 1% to about 99%, or about 1% to about 90% w/w, or about 1% to about 80%, or about 1% to about 70%, or about 1% to about 60%, or about 1% to about 50%, or about 1% to about 40%, or about 1% to about 30%, or about 1% to about 20%, or about 1% to about 10%; or 10% to about 100%, or about 10% to about 90% w/w, or about 10% to about 80%, or about 10% to about 70%, or about 10% to about 60%, or about 10% to about 50%, or about 10% to about 40%, or about 10% to about 30%, or about 10% to about 20%; or 20% to about 100%, or about 20% to about 90% w/w, or about 20% to about 80%, or about 20% to about 70%, or about 20% to about 60%, or about 20% to about 50%, or about 20% to about 40%, or about 20% to about 30%; or 30% to about 100%, or about 30% to about 90% w/w, or about 30% to about 80%, or about 30% to about 70%, or about 30% to about 60%, or about 30% to about 50%, or about 30% to about 40%; or 40% to about 100%, or about 40% to about 90% w/w, or about 40% to about 80%, or about 40% to about 70%, or about 40% to about 60%, or about 40% to about 50%; or 50% to about 100%, or about 50% to about 90% o w/w, or about 50% to about 80%, or about 50% to about 70%, or about 50% to about 60%, or 60% to about 100%, or about 60% to about 90% w/w, or about 60% to about 80%, or about 60% to about 70%, or 70% to about 100%, or about 70% to about 90% w/w, or about 70% to about 80%, or 80% to about 100%, or about 80% to about 90% w/w; or 90% to about 100%; or about 2% to about 9%, or about 3% to about 8%, or about 4% to about 8%, or about 5% to about 7%; or about 5%; or about 7%, or about 15%; or about 40%; or about 50%; or about 60%; or about 70%; or about 85%; or about 90%; or about 95%, including all values and subranges in between. In aspects, the third polymer is a PLGA polymer. In aspects, the PLGA polymer is RG 502 S.

In certain embodiments, the biodegradable polymer matrix includes a third polymer. In aspects, the third polymer comprises as a % w/w of the pharmaceutical composition: about 1% to about 99%, or about 1% to about 90% w/w, or about 1% to about 80%, or about 1% to about 70%, or about 1% to about 60%, or about 1% to about 50%, or about 1% to about 40%, or about 1% to about 30%, or about 1% to about 20%, or about 1% to about 10%; or 10% to about 100%, or about 10% to about 90% w/w, or about 10% to about 80%, or about 10% to about 70%, or about 10% to about 60%, or about 10% to about 50%, or about 10% to about 40%, or about 10% to about 30%, or about 10% to about 20%; or about 15% to about 100%, or about 15% to about 95%, or about 15% to about 90%, or about 15% to about 85%, or about 15% to about 80%, or about 15% to about 70%, or about 15% to about 60%, or about 15% to about 50%, or about 15% to about 40%, or about 15% to about 30%, or about 15% to about 20%, or 20% to about 100%, or about 20% to about 90% w/w, or about 20% to about 80%, or about 20% to about 70%, or about 20% to about 60%, or about 20%to about 50%, or about 20% to about 40%, or about 20% to about 30%; or 30% to about 100%, or about 30% to about 90% w/w, or about 30%to about 80%, or about 30%to about 70%, or about 30% to about 60%, or about 30%to about 50%, or about 30% to about 40%; or 40% to about 100%, or about 40% to about 90% w/w, or about 40% to about 80%, or about 40% to about 70%, or about 40% to about 60%, or about 40% to about 50%; or 50% to about 100%, or about 50% to about 90% w/w, or about 50% to about 80%, or about 50%, to about 70%, or about 50%, to about 60%; or 60% to about 100%, or about 60% to about 90% w/w, or about 60% to about 80%, or about 60% to about 70%; or about 2% to about 9%, or about 3% to about 8%, or about 4% to about 8%, or about 5% to about 7%; including all values and subranges in between. In embodiments, the third polymer is a PLGA polymer. In embodiments, the PLGA polymer is RG 502 S. In aspects, the PLGA polymer can be present as a mixture of polymers in the biodegradable polymer matrix.

In certain embodiments, the biodegradable polymer matrix includes a first polymer, a second polymer, and a third polymer. In aspects, the first polymer, the second polymer, and the third polymer comprise as a % w/w ratio of the pharmaceutical composition about 1%/99% to about 99%/1%, or about 5%/95% to about 95%/5%, or about 10%/90% to about 90%/10%, or about 15%/85% to about 85%/15%, or about 20%/80% to about 80%/20%, or about 25%/75% to about 75%/25%, or about 30%/70% to about 70%/30%, or about 35%/65% to about 65%/35%, or about 40%/60% to about 60%/40%, or about 45%/55% to about 55%/45%, or about 50%/50%.

In embodiments in which the biodegradable polymer matrix includes a first polymer, a second polymer, and a third polymer, said polymers can be present in the biodegradable polymer matrix at the following ratios: from 1:1:1 to 100:1:1 to 1:100:1to 1:1:100; or from 10:1:1 to 1:10:1 to 1:1:10; or from 5:1:1: to 1:5:1 to 1:1:5; or from 2:1:1 to 1:2:1to 1:1:2, including all values and subranges in between.

In certain embodiments, the biodegradable polymer matrix includes a third polymer. In aspects, the third polymer comprises as a weight of the biodegradable polymer matrix: about 1 µg to about 1,000 µg, about 1 µg to about 500 µg, or about 1 µg to about 400 µg, or about 1 µg to about 300 µg, or about 1 µg to about 250 µg, or about 1 µg to about 200 µg, or about 1 µg to about 150 µg, or about 1 µg to about 100 µg, or about 1 µg to about 50 µg, or about 1 µg to about 40 µg, or about 1 µg to about 30 µg, or about 1 µg to about 20 µg, or about 1 µg to about 10 µg, or about 1 to about 5 µg, or about 3 µg to about 9 µg, including all values and subranges in between. In aspects, the third is a PLGA polymer. In aspects, the PLGA polymer is RG 502 S. In aspects, the PLGA polymer can be present as a mixture of polymers in the biodegradable polymer matrix.

In one embodiment, the biodegradable polymer matrix contains a mixture of polymers comprising (i) 7 ± 5% of an ester end-capped biodegradable poly(D,L-lactide-co-glycolide) copolymer having an inherent viscosity at 25° C. in 0.1% w/v CHCl₃ of approximately 0.16 to approximately 0.24 dL/g, (ii) 45 ± 5% of an ester end-capped biodegradable poly(D,L-lactide) homopolymer having an inherent viscosity at 25° C. in 0.1% w/v CHCl₃ of approximately 0.25 to approximately 0.35 dL/g, and (iii) 15 ± 5% an ester end-capped biodegradable poly(D,L-lactide) homopolymer having an inherent viscosity at 25° C. in 0.1% w/v CHCl₃ of approximately 1.8 to approximately 2.2 dL/g.

In certain embodiments, the intracameral implant comprises as a biodegradable polymer matrix content: about 1 µg to about 1000 µg, or about 1 µg to about 900 µg, or about 1 µg to about 800 µg, or about 1 µg to about 700 µg, or about 1 µg to about 600 µg, or about 1 µg to about 500 µg, or about 1 µg to about 450 µg, or about 1 µg to about 400 µg, or about 1 µg to about 350 µg, or about 1 µg to about 300 µg, or about 1 µg to about 250 µg, or about 1 µg to about 200 µg, or about 1 µg to about 150 µg, or about 1 µg to about 100 µg, or about 1 µg to about 90 µg, or about 1 µg to about 80 µg, or about 1 µg to about 70 µg, or about 1 µg to about 60 µg, or about 1 µg to about 50 µg, or about 1 µg to about 40 µg, or about 1 µg to about 30 µg, or about 1 µg to about 20 µg, In certain embodiments, the intracameral implant comprises as a biodegradable polymer matrix content: about 10 µg to about 100 µg, or about 10 µg to about 90 µg, or about 20 µg to about 90 µg, or about 25 µg to about 90 µg, or about 27 µg to about 85 µg, or about 27 µg, or about 85 µg.

In certain embodiments, the therapeutic agent comprises as a % w/w of the intracameral implant composition: about 1% to about 90%, or about 1% to about 80%, or about 1% to about 70%, or about 1% to about 60%, or about 1% to about 55%, or about 1% to about 50%, or about 1% to about 45%, or about 1% to about 40%, or about 1% to about 35%, or about 1% to about 30%, or about 1% to about 25%, or about 1% to about 20%, or about 1% to about 15%, or about 1% to about 10%, or about 1% to about 5%, or about 5% to about 90%, or about 5% to about 80%, or about 5% to about 70%, or about 5% to about 60%, or about 5% to about 55%, or about 5% to about 50%, or about 5% to about 45%, or about 5% to about 40%, or about 5% to about 35%, or about 5% to about 30%, or about 5% to about 25%, or about 5% to about 20%, or about 5% to about 15°0, or about 5% to about 10%, or about 10% to about 90%, or about 10% to about 80%, or about 10% to about 70%, or about 10% to about 60%, or about 10% to about 55%, or about 10% to about 50%; or about 10% to about 45%, or about 10% to about 40%, or about 10% to about 35%, or about 10% to about 30%, or about 10% to about 25%, or about 10% to about 20%, or about 10% to about 15%, or about 15 % to about 90%, or about 15% to about 80%, or about 15% to about 70%, or about 15% to about 60%, or about 15% to about 55%, or about 15% to about 50%, or about 15% to about 45°, or about 15% to about 40%, or about 15% to about 35%, or about 15% to about 30%, or about 15% to about 25%, or about 15% to about 20%, or about 20% to about 90%, or about 20% to about 80%, or about 20% to about 70%, or about 20% to about 60%, or about 20% to about 55%, or about 20% to about 50%; or about 20% to about 45%, or about 20% to about 40%, or about 20% to about 35%, or about 20% to about 30%, or about 20% to about 25%, or about 30% to about 90%, or about 30% to about 80%, or about 30% to about 70%, or about 30% to about 60%, or about 30% to about 55%, or about 30% to about 50%, or about 30% to about 45%or about 30% to about 40%, or about 30% to about 35%, or about 40% to about 90%, or about 40% to about 80%, or about 40% to about 70%, or about 40% to about 60%, or about 40% to about 55%, or about 40% to about 50%, or about 40% to about 45%,or about 45% to about 90%, or about 45% to about 80%, or about 45% to about 75%, or about 45%to about 70%, or about 45% to about 65%, or about 45% to about 60%, or about 45% to about 55%, or about 45% to about 50%,or about 50% to about 90%, or about 50% to about 80%, or about 50% to about 70%, or about 50% to about 60%, or about 50% to about 55%, or about 25% to about 40%, or about 28% to about 35%, or about 30%, to about 33%, or about 39%to about 45%.

In certain embodiments, the intracameral implant composition comprises as a therapeutic agent content: of from about 1 µg to about 1000 µg; or about 1 µg to about 500 µg; or about 1 µg to about 400 µg; or about 1 µg to about 300 µg; or about 1 µg to about 200 µg; or about 1 µg to about 100 µg: or about 1 µg to about 90 µg; or about 1 ug to about 80 µg; or about 1 µg to about 70 µg; or about 1 µg to about 60 µg; or about 1 µg to about 50 µg; or about 1 µg to about 40 ug; or about 1 µg to about 30 µg; or about 1 µg to about 20 µg; or about 1 µg to about 10 µg or about 10 µg to about 100 µg; or about 10 µg to about 50 µg; or about 10 µg to about 35 µg; or about 10 µg to about 31 ug; or about 14 µg to about 26 µg; or about 20 µg to about 40 µg; or about 25 µg to about 35 ug; or about 28 µg to about 31 ug; or about 14 µg; or about 19 µg; or about 26 ug; or about 29 µg; or about 42 µg.

In some embodiments, the ocular implant is a rod-shaped implant comprising a shortest dimension of between about 150 to about 225 µm and a longest dimension of between about 1,500 to about 3,000 µm in length.

In other embodiments, the intracameral implant is a rod-shaped implant selected from the group consisting of a rod-shaped implant having dimensions of about 180 µm × about 132 µm × about 1,418 µm ± 20% of each dimension; a rod-shaped implant having dimensions of about 225 µm × about 225 µm × about 2,925 µm ± 20% of each dimension; a rod-shaped implant having dimensions of about 200 µm x about 200 µm × about 1,500 µm ± 20% of each dimension, a rod-shaped implant having dimensions of about 150 µm × about 150 µm × about 1,500 µm ± 20% of each dimension; a rod-shaped implant having dimensions of about 210 µm × about 200 µm × about 1,500 µm ± 20% of each dimension.

In other embodiments, the intracameral implant is a rod-shaped implant selected from the group consisting of: a rod-shaped implant having dimensions of about 190 µm × about 130 µm × about 1,500 µm ± 10% of each dimension; a rod-shaped implant having dimensions of about 225 µm × about 225 µm × about 2,925 µm ± 10% of each dimension, a rod-shaped implant having dimensions of about 200 µm × about 200 µm × about 1,500 µm ±10% of each dimension; a rod-shaped implant having dimensions of about 150 µm × about 150 µm about 1,500 µm ± 10% of each dimension; a rod-shaped implant having dimensions of about 210 µm x about 200 µm × about 1,500 µm ±10% of each dimension.

In other embodiments, the intracameral implant is a rod-shaped implant selected from the group consisting of: a rod-shaped implant having dimensions of about 190 µm × about 130 µm × about 1,500 µm ±5% of each dimension; a rod-shaped implant having dimensions of about 225 µm × about 225 µm × about 2,925 µm ± 5% of each dimension; a rod-shaped implant having dimensions of about 200 µm × about 200 µm × about 1,500 µm ±5% of each dimension, a rod-shaped implant having dimensions of about 150 µm × about 150 µm × about 1,500 µm ± 5% of each dimension; a rod-shaped implant having dimensions of about 210 µm × about 200 µm × about 1,500 µm ± 5% of each dimension.

In some aspects, the disclosure provides a pharmaceutical composition for treating an ocular condition, wherein the composition is fabricated as a rod-shaped ocular implant having dimensions of 190 µm × 130 µm × 1,500 µm (W x H x L) ±100 µm of each dimension, or a rod-shaped ocular implant having dimensions of 225 µm × 225 µm × 2,925 µm (W x H × L) ± 100 µm of each dimension, or a rod-shaped ocular implant having dimensions of 200 µm × 200 µm × 1500 µm (W × H × L) ± 100 µm of each dimension; or a rod-shaped ocular implant having dimensions of 210 µm × about 200 µm × about 1,500 µm (W × H × L) ± 100 µm of each dimension.

In some aspects, the disclosure provides a pharmaceutical composition for treating an ocular condition, wherein the composition is fabricated as a rod-shaped ocular implant having dimensions of 190 µm × 130 µm × 1,500 µm (W × H × L) ± 50 µm of each dimension, or a rod-shaped ocular implant having dimensions of 225 µm × 225 µm × 2,925 µm (W × H × L) ± 50 µm of each dimension, or a rod-shaped ocular implant having dimensions of 200 µm × 200 µm × 1500 µm (W × H × L) ±50 µm of each dimension; or a rod-shaped ocular implant having dimensions of 210 µm × about 200 µm × about 1,500 µm(W x HL) ± 50 µm of each dimension.

In some aspects, the disclosure provides a pharmaceutical composition for treating an ocular condition, wherein the composition is fabricated as a tod-shaped ocular implant having dimensions of 190 µm × 100 µm × 1,500 µm (W H L) ±40 µm of each dimension, or a rod-shaped ocular implant having dimensions of 225 µm × 225 µm × 2,925 µm (W × H × L) ± 40 µm of each dimension or a rod-shaped ocular implant having dimensions of 200 µm × 200 µm × 1500 µm (W × H × L) ±40 µm of each dimension: or a rod-shaped ocular implant having dimensions of 210 µm × about 200 µm × about 1,500 µm (W × H × L) ± 40 µm of each dimension.

In some aspects, the disclosure provides a pharmaceutical composition for treating an ocular condition, wherein the composition is fabricated as a rod-shaped ocular implant having dimensions of 190 µm × 130 µm ×1,500 µm (W × H × L) ± 30 µm of each dimension, or a rod-shaped ocular implant having dimensions of 225 µm × 225 µm × 2,925 µm (W × H × L) ± 30 µm of each dimension or a rod-shaped ocular implant having dimensions of 200 µm × 200 µm × 1500 µm (W × H × L) ± 30 µm of each dimension; or a rod-shaped ocular implant having dimensions of 210 µm × about 200 µm × about 1,500 µm (W × H × L) ± 30 µm of each dimension.

In some aspects, the disclosure provides a pharmaceutical composition for treating an ocular condition, wherein the composition is fabricated as a rod-shaped ocular implant having dimensions of 190 µm ×130 µm × 1,500 µm (W x H x L) ± 20 µm of each dimension, or a rod-shaped ocular implant having dimensions of 225 µm × 225 µm × 2,925 µm (W × H × L) ± 20 µm of each dimension, or a rod-shaped ocular implant having dimensions of 200 µm × 200 µm × 1500 µm (W × H × L) ± 20 µm of each dimension; or a rod-shaped ocular implant having dimensions of 210 µm × about 200 µm × about 1,500 µm (W × H × L) ± 20 µm of each dimension.

In some aspects, the disclosure provides a pharmaceutical composition for treating an ocular condition, wherein the composition is fabricated as a rod-shaped ocular implant having dimensions of 190 µm × 130 µm × 1,500 µm (W × H × L) ± 10 µm of each dimension, or a rod-shaped ocular implant having dimensions of 225 µm × 225 µm × 2,925 µm (W × H × L) ± 10 µm of each dimension, or a rod-shaped ocular implant having dimensions of 200 µm × 200 µm × 1500 µm (W × H × L) ± 10 µm of each dimension; or a rod-shaped ocular implant having dimensions of 210 µm × about 200 µm × about 1,500 µm (W × H × L) ± 10 µm of each dimension

In some aspects, the disclosure provides a pharmaceutical composition for treating an ocular condition, wherein the composition is fabricated as a rod-shaped ocular implant having dimensions of 190 µm × 130 µm × 1,500 µm (W × H × L) ± 5 µm of each dimension, or a rod-shaped ocular implant having dimensions of 225 µm × 225 µm × 2,925 µm (W × H × L) ± 5 µm of each dimension or a rod-shaped ocular implant having dimensions of 200 µm × 200 µm × 1500 µm (W × H × L) ± 5 µm of each dimension; or a rod-shaped ocular implant having dimensions of 210 µm × about 200 µm × about 1,500 µm (W × H × L) ± 5 µm of each dimension.

In some aspects, the disclosure provides a pharmaceutical composition for treating an ocular condition, wherein the composition is fabricated as a rod-shaped ocular implant having dimensions of 190 µm × 130 µm × 1,500 µm (W × H × L) ± 10% of each dimension, or a rod-shaped ocular implant having dimensions of 225 µm × 225 µm × 2,925 µm (W × H × L) ± 10% of each dimension, or a rod-shaped ocular implant having dimensions of 200 µm × 200 µm × 1500 µm (W × H × L) ± 10% of each dimension; or a rod-shaped ocular implant having dimensions of 210 µm × about 200 µm × about 1,500 µm (W × H × L) ± 10% of each dimension.

In some aspects, the disclosure provides a pharmaceutical composition for treating an ocular condition, wherein the composition is fabricated as a rod-shaped ocular implant having dimensions of 190 µm × 130 µm × 1,500 µm (W × H × L) ± 5% of each dimension, or a rod-shaped ocular implant having dimensions of 225 µm × 225 µm × 2,925 µm (W × H × L) ± 5% of each dimension, or a rod-shaped ocular implant having dimensions of 200 µm × 200 µm × 1500 µm (W × H × L) ± 5% of each dimension or a rod-shaped ocular implant having dimensions of 210 µm × about 200 µm × about 1,500 µm (W × H × L) ± 5% of each dimension.

In embodiments, the implants do not substantially swell after administration to the eye of a patient in need thereof. In particular embodiments, the implant does not swell in any dimension by more than about 20%, about 15%, about 10%, about 9%, about 8%, about 7%, about 6%, about 5%, about 4%, about 3%, about 2%, about 1%. In particular embodiments, the implant does not swell in any dimension by more than about 100 µm, about 90 µm, about 80 µm, about 70 µm, about 60 µm, about 50 µm, about 40 µm, about 30 µm, about 20 µm, about 10 µm, or about 50 µm or less. Thus, when referring to an “intracameral implant that does not substantially swell,” it is meant that said implant does not swell to such a degree that it would be incompatible with the human iridocorneal angle.

Delivery of such implants disclosed herein include delivery through a 27 gauge needle or smaller. In aspects, the needles can be thin-walled or ultra-thin walled.

In one embodied delivery method the needle is a 28 gauge, 29 gauge, 30 gauge, 31 gauge, 32 gauge, 33 gauge, or 34 gauge needle. In aspects, the needles can be thin-walled or ultra-thin walled.

Provided herein are methods for lowering intraocular pressure in a subject in need thereof comprising administering at least one intracameral implant to the anterior chamber of said subject’s eye, wherein a therapeutic agent is released at a concentration below an EC₅₀) calculated for said therapeutic agent when administered without said intracameral implant, whereby the intraocular pressure in said subject’s eye is lowered. In embodiments, said intracameral implant comprises a biodegradable polymer matrix and at least one therapeutic agent homogenously dispersed therein. In embodiments, the intracameral implant achieves a sustained release of said therapeutic agent into the aqueous humor. In embodiments, the therapeutic agent is selected from the group consisting of prostaglandin, prostaglandin analog (e.g., travoprost), prostamide, prostamide analog, and salts, solvates, esters, and prodrugs thereof, and combinations thereof. In embodiments, the therapeutic agent is released at a concentration at least about 10%, at least about 20%, at least about 30%, at least about 40%, at least about 50%, at least about 60%, at least about 70%, at least about 80%, or at least about 90% below the EC₅₀ calculated for said therapeutic agent when administered without said intracameral implant. In embodiments, the intraocular pressure is lowered for at least 7 months. In such embodiments, the intraocular pressure is lowered by about 25% to about 30%, and the lowered intraocular pressure is maintained for at least about 7 months.

In still further embodiments, methods are provided for lowering intraocular pressure in a human subject in need thereof, comprising: administering at least one intracameral implant to the anterior chamber of said subject’s eye, wherein said intracameral implant achieves a prostaglandin concentration in the aqueous humor of about 0.051 nMol/L, and whereby the intraocular pressure in said subject’s eye is lowered. In embodiments, the intraocular pressure is lowered for at least 7 months. In embodiments, the prostaglandin analog concentration in the aqueous humor of about 0.051 nMol/L ±50% is maintained for at least 7 months. In embodiments, the prostaglandin analog concentration in the aqueous humor of about 0.051 nMol/L is achieved within about 10 days after administration (e.g., within about 9 days, or within about 8 days, or within about 7 days, or within about 6 days, or within about 5 days, or within about 4 days, or within about 3 days, or within about 2 days, or within about 1 day). In embodiments, the prostaglandin analog concentration in the aqueous humor fluctuates within ±5%, ±10%, ±15%, or ±20% of 0.051 nMol/L after attaining the concentration of about 0.051 nMol/L. In embodiments, the intraocular pressure is lowered by about 20% to about 50%. In embodiments, the intraocular pressure is lowered by about 20% to about 50%, and wherein the lowered intraocular pressure is maintained for at least 7 months. In embodiments, two intracameral implants are administered to said subject per eye. In such embodiments, the intracameral implants comprise as a travoprost content about 14 µg per implant.

In yet still further embodiments, methods are provided for lowering intraocular pressure in a human subject in need thereof, comprising: administering travoprost to the anterior chamber of said subject’s eye, wherein the travoprost acid concentration in the aqueous humor is about 0.051 nMol/L, and whereby the intraocular pressure in said subject’s eye is lowered. In embodiments, the intraocular pressure is lowered for at least 7 months. In embodiments, the travoprost acid concentration in the aqueous humor of about 0.051 nMol/L ± 50% is maintained for at least 7 months. In embodiments, the travoprost acid concentration in the aqueous humor of about 0.051 nMol/L is achieved with about 10 days after administration of travoprost (e.g., within about 9 days, or within about 8 days, or within about 7 days, or within about 6 days, or within about 5 days, or within about 4 days, or within about 3 days, or within about 2 days, or within about 1 day). In embodiments, the travoprost acid concentration in the aqueous humor fluctuates within ±5%, ±10%, ±15%, or ±20% of 0.051 nMol/L. In embodiments, the intraocular pressure is lowered by about 20% to about 50%. In embodiments, the intraocular pressure is lowered by about 20% to about 50%, and wherein the lowered intraocular pressure is maintained for at least 7 months. In embodiments, travoprost is administered via an intracameral implant. In embodiments, two intracameral implants are administered to said subject per eye. In such embodiments, the intracameral implants comprise as a travoprost content about 14 µg per implant.

In other further embodiments, methods are provided for lowering intraocular pressure in a human subject in need thereof, comprising: administering at least one intracameral implant to the anterior chamber of said subject’s eye, wherein said intracameral implant achieves a prostaglandin concentration in the aqueous humor of about 0.165 nMol/L, and whereby the intraocular pressure in said subject’s eye is lowered. In embodiments, the intraocular pressure is lowered for at least 7 months. In embodiments, the prostaglandin analog concentration in the aqueous humor of about 0.165 nMol/L ±50% is maintained for at least 7 months. In embodiments, the prostaglandin analog concentration in the aqueous humor of about 0.165 nMol/L is achieved within about 1 day after administration. In embodiments, the prostaglandin analog concentration in the aqueous humor fluctuates within ±5%, ±10%, ±15%, ±20%, ±30%, ±40%, or ±50% of 0.165 nMol/L after attaining the concentration of about 0.051 nMol/L. In embodiments, the intraocular pressure is lowered by about 20% to about 50%. In embodiments, the intraocular pressure is lowered by about 20% to about 50%, and wherein the lowered intraocular pressure is maintained for at least 7 months. In embodiments, two intracameral implants are administered to said subject per eye In such embodiments, the intracameral implants comprise as a travoprost content about 14 µg per implant

In still other embodiments, methods are provided herein for lowering intraocular pressure in a human subject in need thereof, comprising: administering travoprost to the anterior chamber of said subject’s eye, wherein a travoprost acid concentration in the aqueous humor of about 0.165 nMol/L, and whereby the intraocular pressure in said subject’s eye is lowered. In embodiments, the intraocular pressure is lowered for at least 7 months. In embodiments, a travoprost acid concentration in the aqueous humor of about 0.165 nMol/L ±50% is maintained for at least 7 months. In embodiments, the travoprost acid concentration in the aqueous humor of about 0.165 nMol/L is achieved with about 1 day after administration. In embodiments, the travoprost acid concentration in the aqueous humor fluctuates within ±5%, ±10%, ±15%, ±20%, ±30%, ±40%, or ±50% of 0.165 nMol/L of after attaining the concentration of about 0.165 nMol/L. In embodiments, the intraocular pressure is lowered by about 20% to about 50%. In embodiments, the intraocular pressure is lowered by about 20% to about 50%, and wherein the lowered intraocular pressure is maintained for at least 7 months. In embodiments, travoprost is administered via an intracameral implant. In embodiments, three intracameral implants are administered to said subject per eye In such embodiments, the intracameral implants comprise as a travoprost content about 14 µg per implant.

In embodiments, the disclosure provides a method for lowering intraocular pressure in a subject’s eye, comprising: administering travoprost to the anterior chamber of said subject’s eye, such that a level of travoprost acid is achieved between about 0.051 nMol/L to about 0.165 nMol/L, wherein the intraocular pressure in said subject’s eye is lowered. In embodiments, the intraocular pressure is lowered by at least about 20%. In embodiments, the level of travoprost acid of about 0.051 nMol/L to about 0.165 nMol/L is achieved within about 1 days after administration to said subject’s eye, wherein the level of travoprost acid fluctuates thereafter, and wherein clinically significant lowering of intraocular pressure is sustained. In embodiments, the travoprost acid concentration in the aqueous humor fluctuates within ±5%, ±10%, ±15%, ±20%, ±30%, ±40%, or ± 50% after attaining the concentration of about 0.051 nMol/L to about 0.165 nMol/L. In embodiments, the travoprost is administered via an intracameral implant.

In embodiments, a method for lowering intraocular pressure in a human subject in need thereof comprises: administering at least one intracameral implant to the anterior chamber of said subject’s eye, wherein said intracameral implant comprises a biodegradable polymer matrix and a prostaglandin analog homogeneously dispersed therein, and wherein said intracameral implant achieves a prostaglandin analog concentration in the aqueous humor of about 0.051 nMol/L to about 0.165 nMol/L, and whereby the intraocular pressure in said subject’s eye is lowered. In such embodiments, the intraocular pressure is lowered by about 20% to about 50%. In embodiments, lowered IOP is maintained for at least about 7 months. In embodiments, the prostaglandin analog is travoprost, and the intracameral implant achieves a travoprost acid concentration in the aqueous humor of about 0.051 nMol/L to about 0.165 nMol/L.

In embodiments, the disclosure provides a method for treating glaucoma in a human subject in need thereof comprising: administering at least one intracameral implant to the anterior chamber of said subject’s eye, wherein said intracameral implant comprises a biodegradable polymer matrix and travoprost homogeneously dispersed therein, and wherein said intracameral implant achieves a travoprost acid concentration in the aqueous humor of about 0.051 nMol/L to about 0.165 nMol/L, and whereby, the intraocular pressure in said subject’s eye is lowered. In such embodiments, the intraocular pressure is lowered by about at least about 20% (e.g., to about 50%). In embodiments, reduced IOP is maintained for at least about 7 months.

In embodiment, the disclosure provides a method for lowering intraocular pressure in a subject’s eye, comprising: administering travoprost to the anterior chamber of said subject’s eye, such that a level of travoprost acid is achieved in the aqueous humor of said subject’s eye, which is at least 8x lower than the EC₅₀ value of travoprost acid on its molecular target, and wherein clinically significant lowering of IOP is sustained. In embodiments, the level of travoprost acid achieved in the aqueous humor is about 28x lower than the EC₅₀ value of travoprost acid on its molecular target. In embodiments, the travoprost is administered via an intracameral implant.

In embodiments, the disclosure provides for a method for lowering intraocular pressure in a subject in need thereof, comprising: administering a sustained-release formulation of at least one intraocular pressure-reducing therapeutic agent to the anterior chamber of said subject’s eye; wherein said sustained-release formulation achieves a sustained release of said therapeutic agent into the aqueous humor, and wherein said therapeutic agent is released at a concentration below an EC₅₀ calculated for said therapeutic agent when administered without said sustained-release formulation, and whereby the intraocular pressure in said subject’s eye is lowered. In some embodiments, the intraocular pressure-reducing therapeutic agent is released at a concentration at least about 10%, at least about 20%, at least about 30%, at least about 40%, at least about 50%, at least about 60%, at least about 70%, at least about 80%, or at least about 90% below the EC₅₀ calculated for said therapeutic agent when administered without said sustained-release formulation. In some embodiments, the intraocular pressure-reducing therapeutic agent is travoprost.

In an aspect, one, two, three, four, five, six, seven, eight, nine, or more implants are provided in the method and are implanted. The plurality of implants may be implanted simultaneously into the eye of a patient, sequentially during the same treatment, or sequentially over a period of time during several treatments. In some aspects, a patient receives yearly implants.

In embodiments, at least one intracameral implant is administered to the anterior chamber of a subject’s eye. In embodiments which entail administering one intracameral implant, said implant comprises as a therapeutic agent content of from 14 µg to about 43 µg. In embodiments which entail administering two intracameral implants, each implant comprises as a therapeutic agent content of from 14 µg to about 43 µg, and the total amount of therapeutic agent administered is from 28 µg to about 86 µg. In embodiments which entail administering 3 intracameral implants, each implant comprises as a therapeutic agent content of from 14 µg to about 43 µg, and the total amount of therapeutic agent administered is from 42 µg to about 129 µg. In embodiments, the therapeutic agent is selected from the group consisting of prostaglandin, prostaglandin analog, prostamide, prostamide analog, and salts, solvates, esters, and prodrugs thereof, and combinations thereof. In a particular embodiment, the therapeutic agent is travoprost.

In another aspect, taught herein is a pharmaceutical composition for treating an ocular condition, comprising: a biodegradable implant comprising a polymer matrix comprising at least one polymer; and a therapeutic agent homogenously dispersed within the polymer matrix; wherein the implant comprises: a length within 10%, 7.5%, 5%, 2.5%, 2%, 1.5%, 1%, 0.5%, 0.25%, 0.1% of 2925 microns; a width within 10%, 7.5%, 5%, 2.5%, 2%, 1.5%, 1%, 0.5%, 0.25%, 0.1% of 225 microns; and a height within 10%, 7.5%, 5%, 2.5%, 2%, 1.5%, 1%, 0.5%, 0.25%, 0.1% of 225 microns. In some aspects, the implant degrades over a period not less than about 1 month, not less than about 2 months, not less than about 3 months, not less than about 4 months, not less than about 5 months, not less than about 6 months, not less than about 7 months in the anterior chamber of the eye. In some embodiments, the implant releases the therapeutic agent for at least about 1 month, at least about 2 months, at least about 3 months, at least about 4 months, at least about 5 months, at least about 6 months, at least about at least about 7 months, thereby maintaining a reduction in intraocular pressure.

In another aspect, taught herein is a pharmaceutical composition for treating an ocular condition, comprising: a biodegradable implant comprising a polymer matrix comprising at least one polymer; and a therapeutic agent homogenously dispersed within the polymer matrix; wherein the implant comprises: a length within 10%, 7.5%, 5%, 2.5%, 2%, 1.5%, 1%, 0.5%, 0.25%, 0.1% of 1500 microns; a width within 10%, 7.5%, 5%, 2.5%, 2%, 1.5%, 1%, 0.5%, 0.25%, 0.1% of 150 microns; and a height within 10%, 7.5%, 5%, 2.5%, 2%, 1.5%, 1%, 0.5%, 0.25%, 0.1% of 190 microns. In some aspects, the implant degrades over a period not less than about 1 month, not less than about 2 months, not less than about 3 months, not less than about 4 months, not less than about 5 months, not less than about 6 months, not less than about 7 months in the anterior chamber of the eye. In some embodiments, the implant releases the therapeutic agent for at least about 1 month, at least about 2 months, at least about 3 months, at least about 4 months, at least about 5 months, at least about 6 months, at least about at least about 7 months, thereby maintaining a reduction in intraocular pressure.

In another aspect, taught herein is a pharmaceutical composition for treating an ocular condition, comprising: a biodegradable implant comprising a polymer matrix comprising at least one polymer; and a therapeutic agent homogenously dispersed within the polymer matrix; wherein the implant comprises: a length within 10%, 7.5%, 5%, 2.5%, 2%, 1.5%, 1%, 0.5%, 0.25%, 0.1% of 1500 microns; a width within 10%, 75%, 5%, 2.5%, 2%, 1.5%, 1%, 0.5%, 0.25%, 0.1% of 210 microns; and a height within 10%, 7.5%, 5%, 2.5%, 2%, 1.5%, 1%, 0.5%, 0.25%, 0.1% of 200 microns. In some aspects, the implant degrades over a period not less than about 1 month, not less than about 2 months, not less than about 3 months, not less than about 4 months, not less than about 5 months, not less than about 6 months, not less than about 7 months in the anterior chamber of the eye. In some embodiments, the implant releases the therapeutic agent for at least about 1 month, at least about 2 months, at least about 3 months, at least about 4 months, at least about 5 months, at least about 6 months, at least about at least about 7 months, thereby maintaining a reduction in intraocular pressure.

Some embodiments entail administering one intracameral implant having a volume of 148,078,125 ± 10% cubic microns to an eye. Other embodiments entail administering two intracameral implants each having a volume of 148,078,125 ± 10% cubic microns to an eye. Yet other embodiments entail administering three intracameral implants each having a volume of 148,078,125 ± 10% cubic microns to an eye. Yet other embodiments entail administering three or more intracameral implants each having a volume of 148,078,125 ± 10% cubic microns to an eye. In some embodiments, each of the aforementioned intracameral implants having a volume of 148,078,125 ± 10% cubic microns contains a travoprost content of about 42.5 µg.

Some embodiments entail administering one intracameral implant having a volume of 37,050,000 ± 10% cubic microns to an eye. Other embodiments entail administering two intracameral implants each having a volume of 37,050,000 ± 10% cubic microns to an eye. Yet other embodiments entail administering three intracameral implants each having a volume of 37,050,000 ± 10% cubic microns to an eye. Yet other embodiments entail administering three or more intracameral implants each having a volume of 37,050,000 ± 10% cubic microns to an eye. In some embodiments, each of the aforementioned intracameral implants having a volume of 37,050,000 ± 10% cubic microns contains a travoprost content of about 14 µg to about 26 µg (e.g., about 14 µg, about 19 µg, or about 26 µg).

Some embodiments entail administering one intracameral implant having a volume of 63,000,000 ± 10% cubic microns to an eye. Other embodiments entail administering two intracameral implants each having a volume of 63,000,000 ± 10% cubic microns to an eye. Yet other embodiments entail administering three intracameral implants each having a volume of 63,000,000 ± 10% cubic microns to an eye. Yet other embodiments entail administering three or more intracameral implants each having a volume of 63,000,000 ± 10% cubic microns to an eye. In some embodiments, each of the aforementioned intracameral implants having a volume of 63,000,000 ± 10% cubic microns contains a travoprost content of about 30 µg to about 50 µg (e.g, about 31 µg or about 40 µg or about 45 µg).

In particular embodiments, the methods provide for lowering intraocular pressure provided herein, comprising administering a administering at least one intracameral implant to the anterior chamber of said subject’s eye, wherein said intracameral implant comprises: A) a biodegradable polymer matrix, and B) at least one therapeutic agent homogenously dispersed therein. In embodiments, the biodegradable polymer matrix contains a mixture of polymers, comprising as a wt % per implant: i) 22 +/- 5% of a biodegradable poly(D,L-lactide) homopolymer having an inherent viscosity of 0.25 to 0.35 dL/g measured at 0.1% w/v in CHCl₃ at 25° C. with a Ubbelhode size 0c glass capillary viscometer, and ii) 45 +/- 5% of a biodegradable poly(D,L-lactide) homopolymer having an inherent viscosity of 1.8 to 2.2 dL/g measured at 0.1% w/v in CHCl₃ at 25° C. with a Ubbelhode size 0c glass capillary viscometer. In embodiments, the therapeutic agent is selected from the group consisting of prostaglandin, prostaglandin analog (e.g., travoprost), prostamide, prostamide analog, and salts, solvates, esters, and prodrugs thereof, and combinations thereof. In embodiments, the therapeutic agent is present in an amount of about 10 µg to about 20 µg per implant. In embodiments, the implant is formulated to reduce intraocular pressure for at least 7 months. In embodiments, the implant is formulated to achieve IOP-lowering by releasing the therapeutic agent at a concentration which is below the EC₅₀ calculated for said therapeutic agent when administered without said intracameral implant (e.g., by about 10%, about 20%, about 30%, about 40%, about 50%, about 60%, about 70%, about 80%, about 90%, or about 95%).

In particular embodiments, the methods provide for lowering intraocular pressure provided herein, comprising administering at least one intracameral implant to the anterior chamber of said subject’s eye, wherein said intracameral implant comprises: A) a biodegradable polymer matrix; and B) travoprost. In embodiments, the biodegradable polymer matrix contains a mixture of polymers, comprising as a wt % per implant: i) 22 +/- 5% of a biodegradable poly(D,L-lactide) homopolymer having an inherent viscosity of 0.25 to 0.35 dL/g measured at 0.1% w/v in CHCl₃ at 25° C. with a Ubbelhode size 0c glass capillary viscometer; and ii) 45 +/- 5% of a biodegradable poly(D,L-lactide) homopolymer having an inherent viscosity of 1.8 to 2.2 dL/g measured at 0.1% w/v in CHCl₃ at 25° C. with a Ubbelhode size 0c glass capillary viscometer. In embodiments, the intracameral implant is about 190 × 130 × 1,500 µm ± 20% of each dimension. In embodiments, the travoprost is present in an amount of about 14.1 µg. In embodiments, the implant is formulated to lower intraocular pressure for at least 7 months. In embodiments, intraocular pressure is lowered by at least about 20% (e.g., to about 50%). In embodiments, the implant is formulated to achieve IOP-lowering by releasing the travoprost at a concentration which is below the EC₅₀ calculated for travoprost when administered without said intracameral implant. In embodiments, travoprost is released at a concentration at least about 10%, at least about 20%, at least about 30%, at least about 40%, at least about 50%, at least about 60%, at least about 70%, at least about 80%, or at least about 90% below the EC₅₀ calculated for travoprost when administered without said intracameral implant. In embodiments, the intracameral implant is formulated to achieve a travoprost acid concentration in the aqueous humor of about 0.0327 nMol/L to about 0.380 nMol/L (e g., 0.051 nMol/L to about 0.165 nMol/L)

In particular embodiments, the methods provide for lowering intraocular pressure provided herein, comprising administering two intracameral implants to the anterior chamber of said subject’s eye, wherein said intracameral implant comprises A) a biodegradable polymer matrix; and B) travoprost. In embodiments, the biodegradable polymer matrix contains a mixture of polymers, comprising as a wt % per implant: i) 22 +/- 5% of a biodegradable poly(D,L-lactide) homopolymer having an inherent viscosity of 0.25 to 0.35 dL/g measured at 0.1% w/v in CHCl₃ at 25° C. with a Ubbelhode size 0c glass capillary viscometer; and ii) 45 +/- 5% of a biodegradable poly(D,L-lactide) homopolymer having an inherent viscosity of 1.8 to 2.2 dL/g measured at 0.1% w/v in CHCl₃ at 25° C. with a Ubbelhode size 0c glass capillary viscometer. In embodiments, the intracameral implant is about 190 × 130 × 1,500 µm ± 20% of each dimension. In embodiments, the travoprost is present in an amount of about 14.1 µg per implant (28.2 µg total). In embodiments, the implant is formulated to lower intraocular pressure for at least 7 months. In embodiments, intraocular pressure is lowered by about at least about 20% (e.g., to about 50%). In embodiments, the implant is formulated to achieve IOP-lowering by releasing the travoprost at a concentration which is below the EC₅₀ calculated for travoprost when administered without said intracameral implant. In embodiments, travoprost is released at a concentration at least about 10%, at least about 20%, at least about 30%, at least about 40%, at least about 50%, at least about 60%, at least about 70%, at least about 80%, or at least about 90% below the EC50 calculated for travoprost when administered without said intracameral implant. In embodiments, the intracameral implant is formulated to achieve a travoprost acid concentration in the aqueous humor of about 0.0327 nMol/L to about 0.380 nMol/L (e.g. about 0.051 nMol/L to about 0.165 nMol/L).

In particular embodiments, the methods provide for lowering intraocular pressure provided herein, comprising administering three intracameral implants to the anterior chamber of said subject’s eye, wherein said intracameral implant comprises: A) a biodegradable polymer matrix; and B) travoprost. In embodiments, the biodegradable polymer matrix contains a mixture of polymers, comprising as a wt % per implant: i) 22 +/- 5% of a biodegradable poly(D,L-lactide) homopolymer having an inherent viscosity of 0.25 to 0.35 dL/g measured at 0.1% w/v in CHCl₃ at 25° C. with a Ubbelhode size 0c glass capillary viscometer; and ii) 45 +/- 5% of a biodegradable poly(D,L-lactide) homopolymer having an inherent viscosity of 1.8 to 2.2 dL/g measured at 0.1% w/v in CHCl₃ at 25° C. with a Ubbelhode size 0c glass capillary viscometer. In embodiments, the intracameral implant is about 190 × 150 × 1,500 µm ± 20% of each dimension. In embodiments, the travoprost is present in an amount of about 14-26 µg per implant (about 28 µg to about 52 µg total dose). In embodiments, the implant is formulated to lower intraocular pressure for at least 7 months. In embodiments, intraocular pressure is lowered by about 15% to about 50% (e.g., at least about 20%). In embodiments, the implant is formulated to achieve IOP-lowering by releasing the travoprost at a concentration which is below the EC₅₀ calculated for travoprost when administered without said intracameral implant. In embodiments, travoprost is released at a concentration at least about 10%, at least about 20%, at least about 30%, at least about 40%, at least about 50%, at least about 60%, at least about 70%, at least about 80%, or at least about 90% below the EC50 calculated for travoprost when administered without said intracameral implant. In embodiments, the intracameral implant is formulated to achieve a travoprost acid concentration in the aqueous humor of about 0.0327 nMol/L to about 0.380 nMol/L (e.g., about 0.051 nMol/L to about 0.165 nMol/L)

In particular embodiments, the methods provide for lowering intraocular pressure provided herein, comprising administering two intracameral implants to the anterior chamber of said subject’s eye, wherein said intracameral implant comprises: A) a biodegradable polymer matrix; and B) travoprost. In embodiments, the biodegradable polymer matrix contains a mixture of polymers, comprising as a wt % per implant: i) 22 +/- 5% of a biodegradable poly(D,L-lactide) homopolymer having an inherent viscosity of 0.25 to 0.35 dL/g measured at 0.1% w/v in CHCl₃ at 25° C. with a Ubbelhode size 0c glass capillary viscometer; and ii) 45 +/- 5% of a biodegradable poly(D,L-lactide) homopolymer having an inherent viscosity of 1.8 to 2.2 dL/g measured at 0.1% w/v in CHCl₃ at 25° C. with a Ubbelhode size 0c glass capillary viscometer. In embodiments, the intracameral implant is about 200 × 200 × 1,500 µm ± 20% of each dimension. In embodiments, the travoprost is present in an amount of about 14-26 µg per implant (about 28 µg to about 52 µg total dose). In embodiments, the implant is formulated to lower intraocular pressure for at least 7 months. In embodiments, intraocular pressure is lowered by about 15% to about 50% (e.g., about 20% to about 30%). In embodiments, the implant is formulated to achieve IOP-lowering by releasing the travoprost at a concentration which is below the EC₅₀ calculated for travoprost when administered without said intracameral implant. In embodiments, travoprost is released at a concentration at least about 10%, at least about 20%, at least about 30%, at least about 40%, at least about 50%, at least about 60%, at least about 70%, at least about 80%, or at least about 90% below the EC₅₀ calculated for travoprost when administered without said intracameral implant. In embodiments, the intracameral implant is formulated to achieve a travoprost acid concentration in the aqueous humor of about 0.0327 nMol/L to about 0.380 nMol/L (e.g., about 0.051 nMol/L to about 0.165 nMol/L).

In particular embodiments, the methods provide for lowering intraocular pressure provided herein, comprising administering at least one intracameral implants to the anterior chamber of said subject’s eye, wherein said intracameral implant comprises: A) a biodegradable polymer matrix; and B) travoprost. In embodiments, the biodegradable polymer matrix contains a mixture of polymers, comprising as a wt % per implant. i) 9 ± 5% of an ester end-capped biodegradable poly(D,L-lactide-co-glycolide) co-polymer having an inherent viscosity of approximately 0.8 to approximately 1.2 dL/g as measured at 25° C. in 0.1% w/v CHCl₃ and ii) 48 ± 5 % of an ester end-capped biodegradable poly(D,L-lactide) homopolymer having an inherent viscosity of approximately 1.8 to approximately 2.2 dL/g as measured at 25° C. in 0.1% w/v CHCl₃. In embodiments, the intracameral implant is about 200 × 200 × 1,500 µm ± 20% of each dimension. In embodiments, the travoprost is present in an amount of about 28-31 µg per implant. In embodiments, the implant is formulated to lower intraocular pressure for at least 7 months. In embodiments, intraocular pressure is lowered by about 15% to about 50% (e.g, at least about 20%). In embodiments, the implant is formulated to achieve IOP-lowering by releasing the travoprost at a concentration which is below the EC₅₀ calculated for travoprost when administered without said intracameral implant. In embodiments, travoprost is released at a concentration at least about 10%, at least about 20%, at least about 30%, at least about 40%, at least about 50%, at least about 60%, at least about 70%, at least about 80%, or at least about 90% below the EC₅₀ calculated for travoprost when administered without said intracameral implant.

In particular embodiments, the methods provide for lowering intraocular pressure provided herein, comprising administering at least two intracameral implants to the anterior chamber of said subject’s eye, wherein said intracameral implant comprises: A) a biodegradable polymer matrix; and B) travoprost. In embodiments, the biodegradable polymer matrix contains a mixture of polymers, comprising as a wt % per implant: i) 9 ± 5% of an ester end-capped biodegradable poly(D,L-lactide-co-glycolide) co-polymer having an inherent viscosity of approximately 0.8 to approximately 1.2 dL/g as measured at 25° C. in 0.1% w/v CHCl₃ and ii) 48 ± 5 % of an ester end-capped biodegradable poly(D,L-lactide) homopolymer having an inherent viscosity of approximately 1.8 to approximately 2 2 dL/g as measured at 25° C. in 0 1% w/v CHCl₃. In embodiments, the intracameral implant is about 200 × 200 × 1,500 µm ± 20% of each dimension In embodiments, the travoprost is present in an amount of about 31 µg per implant (for a total dose of 62 µg). In embodiments, the implant is formulated to lower intraocular pressure for at least 7 months. In embodiments, intraocular pressure is lowered by about 15% to about 50% (e.g., about 20% to about 30%). In embodiments, the implant is formulated to achieve IOP-lowering by releasing the travoprost at a concentration which is below the EC₅₀ calculated for travoprost when administered without said intracameral implant. In embodiments, travoprost is released at a concentration at least about 10%, at least about 20%, at least about 30%, at least about 40%, at least about 50%, at least about 60%, at least about 70%, at least about 80%, or at least about 90% below the EC₅₀ calculated for travoprost when administered without said intracameral implant.

In particular embodiments, the methods provide for lowering intraocular pressure provided herein, comprising administering at least one intracameral implant (e.g., two or more) to the anterior chamber of said subject’s eye, wherein said intracameral implant comprises: A) a biodegradable polymer matrix; and B) travoprost. In embodiments, the biodegradable polymer matrix contains a mixture of polymers, comprising as a wt % per implant. (i) 7 ± 5% of an ester end-capped biodegradable poly(D,L-lactide-co-glycolide) copolymer having an inherent viscosity at 25° C. in 0.1% w/v CHCl₃ of approximately 0.16 to approximately 0.24 dL/g, (ii) 45 ± 5% of an ester end-capped biodegradable poly(D,L-lactide) homopolymer having an inherent viscosity at 25° C. in 0.1% w/v CHCl₃ of approximately 0.25 to approximately 0.35 dL/g, and (iii) 15 ± 5% of an ester end-capped biodegradable poly(D,L-lactide) homopolymer having an inherent viscosity at 25° C. in 0.1% w/v CHCl₃ of approximately 1.8 to approximately 2.2 dL/g In embodiments, the intracameral implant is about 200 × 200 × 1.500 µm ± 20% of each dimension. In embodiments, the travoprost is present in an amount of about 14.7 µg per implant (a total dose of 29.4 µg in embodiments in which two implants are administered). In embodiments, the implant is formulated to lower intraocular pressure for at least 7 months. In embodiments, intraocular pressure is lowered by about 15% to about 50% (e.g., about 20% to about 30%). In embodiments, the implant is formulated to achieve IOP-lowering by releasing the travoprost at a concentration which is below the EC₅₀ calculated for travoprost when administered without said intracameral implant In embodiments, travoprost is released at a concentration at least about 10%, at least about 20%, at least about 30%, at least about 40%, at least about 50%, at least about 60%, at least about 70%, at least about 80%, or at least about 90% below the EC₅₀ calculated for travoprost when administered without said intracameral implant.

In embodiments, a rod-shaped mold having dimensions of 215 × 230 × 2,925 µm (W × H × L) is used to fabricate an implant having dimensions of 175 × 215 × 2,780 µm (W × H × L). In embodiments, a rod-shaped mold having dimensions of 145 × 190 × 1,500 µm (W × H × L) is used to fabricate an implant having dimensions of 132 × 180 × 1,438 µm (W × H × L). In embodiments, a rod-shaped mold having dimensions of 210 × 220 × 1,550 µm (W × H × L) is used to fabricate an implant having dimensions of 200 × 190 × 1,500 µm (W × H × L). In embodiments, a rod-shaped mold having dimensions of 175 × 215 × 1,390 µm (W × H × L) is used to fabricate an implant having dimensions of 170 × 210 × 1,325 µm (W × H × L).

In certain embodiments, the intracameral implant is ENV515-3, ENV515-3-2, ENV-515-4/5, or ENV515-16-2.

### BRIEF DESCRIPTION OF THE DRAWINGS

FIG. 1A is a schematic of the anatomy of a human eye. FIG. 1B is a schematic of an intracameral implant placed in the iridocorneal angle of the eye and also a depiction of the aqueous humor outflow located in the iridocorneal angle of the eye.

FIG. 2 illustrates the design of the Phase 2a clinical study for the ENV515-3 (travoprost) intracameral implants.

FIGS. 3A and 3B graphically illustrate the IOP measurements acquired during the Phase 2a clinical studies. The y-axis shows the IOP measurements in mmHg at different time points shown in the x-axis FIG. 3A graphically illustrates the median IOP measured for each ocular treatment FIG. 3B graphically illustrates the median IOP measurements adjusted to establish a baseline during the post washout period for each ocular treatment.

FIG. 4A graphically illustrates the diurnal IOP measurement at day 25 of the study. The x-axis shows the three time points (8 AM, 10 AM, and 4 PM) at which IOP was measured for each ocular treatment. The y-axis shows the median IOP measurements as a percent change from the baseline. FIG. 4B and FIG. 4C illustrate the average, and percent change from baseline, in diurnal IOP average (Average of 8 AM, 10 AM, and 4 PM IOPs), respectively. FIG. 4D illustrates change from baseline in time-matched diurnal IOP at 8 AM, 10 AM and 4 PM. FIGS. 4E and 4F illustrate the average 8 AM IOP and percent change from baseline in 8 AM IOP, respectively. The figure, as well as others herein, contains the legend “low dose” and “high dosc.” The legend has the following meaning throughout the specification and figures: ENV515-3 low dose is 2 implants per eye. ENV515-3 high dose is 3 implants per eye ENV515-1 low dose is 1 implant per eye. ENV515-1 high dose is 2 implants per eye.

FIG. 5 graphically illustrates the concentration of travoprost acid (nMol/L) in the aqueous humor (shown on the y-axis) responsible for lowering IOP as measured for 2x ENV515-3 (14.1 µg total travoprost in two intraocular implants), 3x ENV515-3 (28.2 µg total travoprost in three intraocular implants), and TRAVATAN Z® eye drops. Also shown in FIG. 5 is the EC₅₀ of travoprost acid for the prostaglandin F (FP) receptor when administered using TRAVATAN Z® eye drops, and this indicates the concentration of free travoprost acid needed to inhibit half of the maximum IOP

FIGS. 6A and 6B illustrate the mean hyperemia score and change from baseline in hyperemia score for study participants, respectively.

FIG. 7A illustrates the aqueous humor travoprost acid levels of study participants. FIG. 7B illustrates mean hyperemia scores of study participants.

FIG. 8A illustrates the mean recovered implant travoprost ester concentration. FIG. 8B illustrates the mean recovered implant travoprost acid concentration.

FIG. 9 illustrates the ENV515 Phase 2a Cohort 2 study design, which was designed to assess long term safety and efficacy of low dose ENV51 5-3 (2 implants/eye).

FIG. 10A illustrates 6 month 8 AM IOP values. FIG. 10B illustrates 6 month diurnal IOP values. FIG. 10C illustrates an individual IOP plot measured for patent 212 over 168 days. FIG. 10D illustrates an individual IOP plot measured for patent 214 over 168 days FIG. 10E illustrates an individual IOP plot measured for patent 215 over 168 days. FIG. 10F illustrates an individual IOP plot measured for patent 231 over 168 days FIG. 10G illustrates an individual diurnal IOP plot measured for patent 212 over 24 weeks. FIG. 10H illustrates an individual diurnal IOP plot measured for patent 231 over 24 weeks. FIG. 10I illustrates an individual diurnal IOP plot measured for patent 214 over 24 weeks. FIG. 10J illustrates an individual diurnal IOP plot measured for patent 215 over 24 weeks FIG. 10K illustrates 6 month 8 AM and diurnal IOP values

FIG. 11A illustrates 7 month 8 AM IOP values. FIG. 11B illustrates 6 month diurnal TOP values

FIG. 12A illustrates ENV515 Ph2a Cohort 2 interim analysis of hyperemia score measured for the ENV515-3 implants. FIG. 12D illustrates ENV515 Ph2a Cohort 2 interim analysis of hyperemia score in terms of a change in baseline measured for the ENV515-3 implants.

FIG. 13A illustrates gonioscopy image analysis of implant orientation at day 42 for subject 214 and subject 215. FIG. 13B illustrates gonioscopy image analysis of implant orientation at 4 months for subject 214 and subject 215.

FIG. 14A illustrates corneal thickness measured for 168 days after administration of ENV515-3 implants FIG. 14B illustrates mean endothelial cell count measured for 180 days after administration of ENV515-3 implants

FIG. 15A illustrates in-vitro release of travoprost (µg) from an ENV515-16-2 implant (ENV-1G-167-16-2). FIG. 15B illustrates in-vitro release of travoprost (%) from an ENV515-16-2 implant (ENV-1G-167-16-2) FIG. 15C illustrates in-vitro release rate of travoprost from an ENV-515-16-2 implant (ENV-1G-167-16-2). FIG. 15D illustrates in-vitro release of travoprost (µg) from ENV515-4/5 implants FIG. 15E illustrates in-vitro release of travoprost (%) from ENV515-4/5 implants FIG. 15F illustrates in-vitro release rate of travoprost from ENV515-4/5 implants.

FIG. 16 depicts optical images of implants captured in an in-vitro travoprost release assay measured for ENV515-16-2 at the following time points (A) two weeks; (B) 4 weeks, and (C) 8 weeks, and for ENV515-5-4/5 measured at the following points: (D) two weeks; (E) 8 weeks; (F) 12 weeks, and (G) 14 weeks FIG. 16H depicts a gonioscopy image from a beagle dog IOP study obtained at day 14.

FIG. 17A illustrates ENV515-3 average in-vitro daily release of travoprost (ng/day) over 140 days. FIG. 17B illustrates ENV515-3 average in-vitro release of travoprost (%) over 140 days. FIG. 17C illustrates ENV515-3 IOP lowering measured with ENV515-3 over 196 days compared to Timolol administered daily.

FIG. 18 illustrates IOP lowering as measured with ENV515-4 implants (1 implant/eye and 2 implants/eye).

FIG. 19A illustrates in-vitro travoprost release (ng/day) from ENV515-3-2 implants, batch 29A. FIG. 19B illustrates in-vitro travoprost release (%) from ENV515-3-2 implants, batch 29-A FIG. 19C illustrates in-vitro travoprost release (ng/day) from ENV515-3-2 implants, batch 16087. FIG. 19D illustrates in-vitro travoprost release (%) from ENV515-3-2 implants, batch 16087.

FIG. 20 illustrates greater than 7 month IOP lowering observed in a beagle dog model utilizing a ENV515-3-2 implant.

FIG. 21 illustrates greater than 7 month IOP lowering observed in a beagle dog model utilizing a ENV515-3-1 implant.

### DETAILED DESCRIPTION

Provided herein are pharmaceutical compositions for treating an ocular condition. In embodiments, the pharmaceutical composition comprises: a biodegradable polymer matrix and a therapeutic agent, which is included in the polymer matrix. In embodiments, the therapeutic agent is dispersed homogeneously throughout the polymer matrix.

As described herein, multiple pharmaceutical compositions have been fabricated and/or contemplated in the form of an implant, resulting in highly effective pharmaceutically active products including ocular therapeutic treatments including sustained release ocular implants.

In various embodiments, these pharmaceutical compositions include a therapeutic agent dispersed throughout a polymer matrix formed into an ocular implant

In a particular embodiment, the pharmaceutical composition of the present disclosure comprises: i) a biodegradable polymer or blend of biodegradable polymers, and ii) a therapeutic agent such as, for example, a drug effective for use in the treatment of an ocular condition, such as elevated intraocular pressure (IOP).

### Definitions

“About” means plus or minus a percent (e.g., ±1%, ±5%, and ±10%) of the number, parameter, or characteristic so qualified, which would be understood as appropriate by a skilled artisan to the scientific context in which the term is utilized. Furthermore, since all numbers, values, and expressions referring to quantities used herein, are subject to the various uncertainties of measurement encountered in the art, and then unless otherwise indicated, all presented values may be understood as modified by the term “about.”

As used herein, the articles “a,” “an,” and “the” may include plural referents unless otherwise expressly limited to one-referent, or if it would be obvious to a skilled artisan from the context of the sentence that the article referred to a singular referent.

Where a numerical range is disclosed herein, then such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include any and all subranges between the minimum value of 1 and the maximum value of 10. Exemplary subranges of the range “1 to 10” include, but are not limited to, 1 to 6.1, 3.5 to 7.8, and 5.5 to 10.

As used herein, the term “polymer” is meant to encompass both homopolymers (polymers having only one type of repeating unit) and copolymers (a polymer having more than one type of repeating unit).

“Biodegradable polymer” means a polymer or polymers, which degrade in vivo, under physiological conditions. The release of the therapeutic agent occurs concurrent with, or subsequent to, the degradation of a biodegradable polymer over time.

The terms “biodegradable” and “bioerodible” are used interchangeably herein. A biodegradable polymer may be a homopolymer, a copolymer, or a polymer comprising more than two different polymeric units.

As used herein, the term “polymer matrix” refers to a homogeneous mixture of polymers. In other words, the matrix does not include a mixture wherein one portion thereof is different from the other portion by ingredient, density, and etc. For example, the matrix does not include a composition containing a core and one or more outer layers, nor a composition containing a drug reservoir and one or more portions surrounding the drug reservoir. The mixture of polymers may be of the same type, e.g. two different PLA polymers, or of ditterent types, e.g. PLA polymers combined with PLGA polymers.

“Ocular condition” means a disease, ailment, or condition, which affects or involves the ocular region

The term “hot-mett extrusion” or “hot-melt extruded” is used herein to describe a process, whereby a blended composition is heated and/or compressed to a molten (or softened) state and subsequently forced through an orifice, where the extruded product (extrudate) is formed into its final shape, in which it solidifies upon cooling.

The term “non-extruded implant” or “non-hot melt extruded implant” refers to an implant that was not manufactured in a process that utilizes an extrusion step, for example, the implant may be made through molding in a mold cavity

“Sustained release” or “controlled release” refers to the release of at least one therapeutic agent, or drug, from an implant at a sustained rate. Sustained release implies that the therapeutic agent is not released from the implant sporadically, in an unpredictable fashion. The term “sustained release” may include a partial “burst phenomenon” associated with deployment. In some example embodiments, an initial burst of at least one therapeutic agent may be desirable, followed by a more gradual release thereafter. The release rate may be steady state (commonly referred to as “timed release” or zero order kinetics), that is the at least one therapeutic agent is released in even amounts over a predetermined time (with or without an initial burst phase), or may be a gradient release. For example, sustained release can have substantially constant release over a given time period or as compared to topical administration

“Therapeutically effective amount” means a level or amount of a therapeutic agent needed to treat an ocular condition; the level or amount of a therapeutic agent that produces a therapeutic response or desired effect in the subject to which the therapeutic agent was administered Thus, a therapeutically effective amount of a therapeutic agent, such as a travoprost, is an amount that is effective in reducing at least one symptom of an ocular condition.

As used herein, the term “baseline” refers to a proper reference measurement established prior to surgery. The baseline measurement can be obtained by any suitable method. In embodiments, “baseline” refers intraocular pressure measured prior to administration of an implant

### Ocular Anatomy

In particular embodiments, the implants described herein are intracameral implants manufactured for placement at or into the iridocorneal angle of the human eye.

In these embodiments, the sustained release of therapeutic agent from the implant achieves a concentration of drug in the aqueous humor of the patient’s eye that significantly lowers IOP over the period of sustained release. Furthermore, in embodiments, the intracameral implant placed at or into the iridocorneal angle of a patient’s eye achieves a drug concentration in the aqueous humor that does not fluctuate below a therapeutic level for any consecutive period of 48 hours or more over the sustained release period of the implant and thus overcomes an inherent problem associated with a topical administration paradigm and prior art implants. In some embodiments, the therapeutic level achieved by the sustained release of a PGA via the intracameral implants described herein may be lower than the therapeutic level achieved using traditional topically administered eye drops.

The anterior and posterior chambers of the eye are filled with aqueous humor, a fluid predominantly secreted by the ciliary body with an ionic composition similar to the blood. The function of the aqueous humor is: a) to supply nutrients to the avascular structures of the eye, e.g the lens and cornea, and b) to maintain IOP.

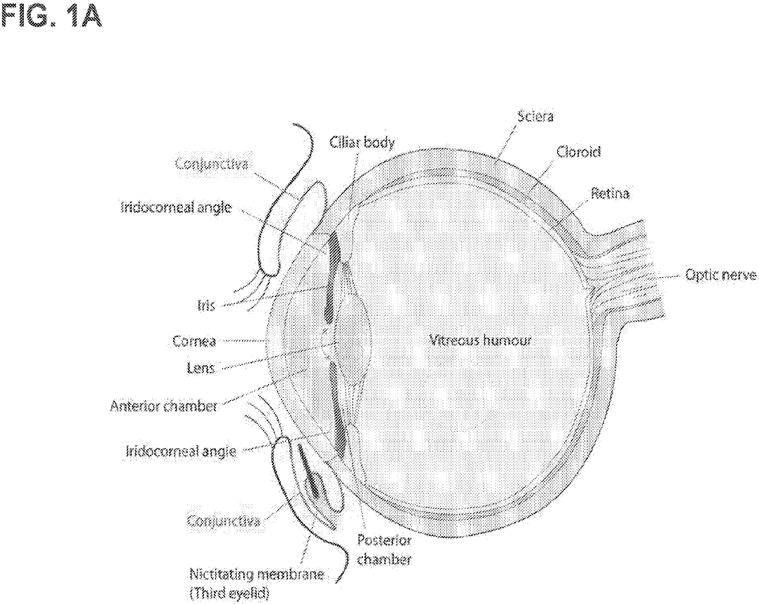

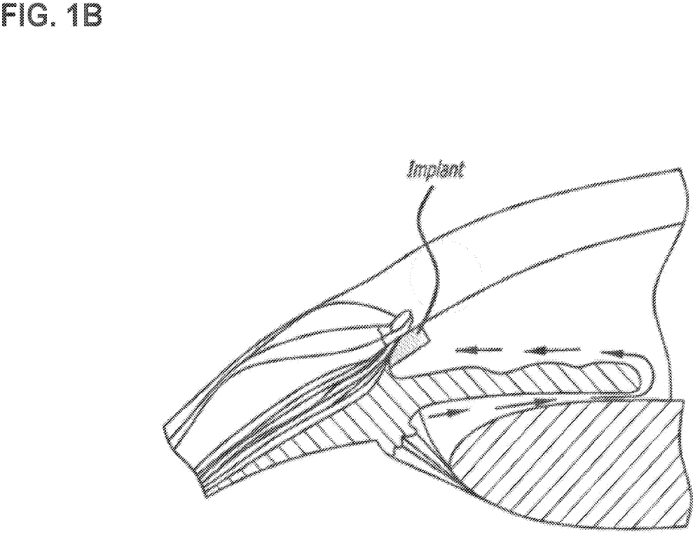

Aqueous humor is predominantly secreted to the posterior chamber of the eye by the ciliary processes of the ciliary body and a minor mechanism of aqueous humor production is through ultrafiltration from arterial blood Aqueous humor reaches the anterior chamber by crossing the pupil and there are convection currents where the flow of aqueous humor adjacent to the iris is upwards, and the flow of aqueous humor adjacent to the cornea flows downwards (FIG. 1B).

There are two different pathways of aqueous humor outflow, both located in the iridocorneal angle of the eye (FIG. 1). The uveoscleral, or nonconventional pathway, refers to the aqueous humor leaving the anterior chamber by diffusion through intercellular spaces among ciliary muscle fibers. Although this seems to be a minority outflow pathway in humans, the uveoscleral pathway is the target of specific antihypertensive drugs, such as the hypotensive lipids.

The aqueous humor drains 360° into the trabecular meshwork that initially has pore size diameters ranging from 10 to under 30 microns in humans. Aqueous humor drains through Schlemm’s canal and exits the eye through 25 to 30 collector channels into the aqueous veins, and eventually into the episcleral vasculature and veins of the orbit.

Therapeutic agent eluting from an implant as described herein enters the aqueous humor of the anterior chamber via convection currents. The therapeutic agent is then dispersed throughout the anterior chamber and enters the target tissues such as the trabecular meshwork and the ciliary body region through the iris root region

Both in the aforementioned trabecular meshwork and in the uveoscleral tissue, various prostanoid receptors have been found, which indicates that prostanoids are invoked in the regulation of aqueous humor production and/or drainage and thereby influence the intraocular pressure. In the trabecular network, genes encoding the EP, FP. IP, DP and TP receptor families are expressed, whereas the EP and FP receptor families are dominant in the uveoscleral tissue (Toris et al., Surv Ophthalmol 2008; 53. Suppl. I. S107-S120).

Prostanoids are physiological fatty acid derivatives representing a subclass of eicosanoids They comprise prostaglandins, prostaniidcs, thromboxanes, and prostacyclins, all of which compounds are mediators involved in numerous physiological processes Natural prostaglandins such as PGF₂ₙ, PGE₂, PGD_{2,} and PGI₂ exhibit a particular affinity to their respective receptors (FP, EP, DP, IP), but also have some non-selective affinity for other prostaglandin receptors Prostaglandins also have direct effects on matrix metalloproteinases. These are neutral proteinases expressed in the trabecular meshwork, which play a role in controlling humor outflow resistance by degrading the extracellular matrix.

Several prostaglandin analogues have been found effective as topically administered medicines in reducing the intraocular pressure, such as latanoprost, bimatoprost, tafluprost, travoprost, and unoprostone By some experts, bimatoprost is understood as a prostamide rather than prostaglandin derivative. Latanoprost, travoprost, tafluprost, and probably also bimatoprost, are potent and selective PGF₂ₙ agonists Their net effect is a reduction of intraocular pressure, which is predominantly caused by a substantial increase in aqueous humor drainage, via the uveoscleral pathway. Probably they also increase the trabecular outflow to some degree. Unoprostone is sometimes also classified as a PGF₂ₙ analogue even though its potency and selectivity are much lower than in the case of the previously mentioned compounds. It is most closely related to a pulmonary metabolite of PGF₂ₙ It is also capable of reducing the intraocular pressure, but appears to act predominantly by stimulating the trabecular drainage pathway, whereas it has little effect on the uveoscleral outflow.

An advantage of injection and intracamcral placement of a biodegradable implant described herein is that the anterior chamber is an immune privileged site in the body and less likely to react to foreign material, such as polymeric therapeutic agent delivery systems.

### Biodegradable Polymers

In certain embodiments, the implants described herein are engineered in size, shape, composition, and combinations thereof, to provide maximal approximation of the implant to the iridocorneal angle of a human eye. In certain embodiments, the implants are made of polymeric materials.

In embodiments, the polymer materials used to form the implants described herein are biodegradable. In embodiments, the polymer materials may be any combination of polylactic acid, glycolic acid, and co-polymers thereof that provides sustained-release of the therapeutic agent into the eye over time.

Suitable polymeric materials or compositions for use in the implants include those materials which are compatible, that is biocompatible, with the eye so as to cause no substantial interference with the functioning or physiology of the eye. Such polymeric materials may be biodegradable, bioerodible or both biodegradable and bioerodible.

In particular embodiments, examples of useful polymeric materials include, without limitation, such materials derived from and/or including organic esters and organic ethers, which when degraded result in physiologically acceptable degradation products. Also, polymeric materials derived from and/or including, anhydrides, amides, orthoesters and the like, by themselves or in combination with other monomers, may also find use in the present disclosure The polymeric materials may be addition or condensation polymers. The polymeric materials may be cross-linked or non-cross-linked For some embodiments, besides carbon and hydrogen, the polymers may include at least one of oxygen and nitrogen The oxygen may be present as oxy, e.g. hydroxy or ether, carbonyl, e.g non-oxo-carbonyl, such as carboxylic acid ester, and the like. The nitrogen may be present as amide, cyano and amino.

In one embodiment, polymers of hydroxyaliphatic carboxylic acids, either homopolymers or copolymers, and polysaccharides are useful in the implants. Polyesters can include polymers of D-lactic acid, L-lactic acid, racemic lactic acid, glycolic acid, polycaprolactone, co-polymers thereof, and combinations thereof.

Some characteristics of the polymers or polymeric materials for use in embodiments of the present disclosure may include: biocompatibility; compatibility with the selected therapeutic agent; ease of use of the polymer in making the therapeutic agent delivery systems described herein, a desired half-life in the physiological environment; and hydrophilicity.

In one embodiment, the biodegradable polymer matrix used to manufacture the implant is a synthetic aliphatic polyester, for example, a polymer of lactic acid and/or glycolic acid, and includes poly-(D,L-lactide) (PLA), poly-(D-lactide), poly-(L-lactide), polyglycolic acid (PGA), and/or the copolymer poly-(D, L-lactidc-co-glycolide) (PLGA).

PLGA and PLA polymers are known to degrade via backbone hydrolysis (bulk erosion) and the final degradation products are lactic and glycolic acids, which are non-toxic and considered natural metabolic compounds. Lactic and glycolic acids are eliminated safely via the Krebs cycle by conversion to carbon dioxide and water.

PLGA is synthesized through random ring-opening co-polymerization of the cyclic dimers of glycolic acid and lactic acid. Successive monomeric units of glycolic or lactic acid are linked together by ester linkages. The ratio of lactide to glycolide can be varied, altering the biodegradation characteristics of the product. By altering the ratio it is possible to tailor the polymer degradation time. Importantly, drug release characteristics are affected by the rate of biodegradation, molecular weight, and degree of crystallinity in drug delivery systems. By altering and customizing the biodegradable polymer matrix, the drug delivery profile can be changed.

PLA, PGA, and PLGA are cleaved predominantly by non-enzymatic hydrolysis of its ester linkages throughout the polymer matrix, in the presence of water in the surrounding tissues. PLA, PGA, and PLGA polymers are biodegradable, because they undergo hydrolysis in the body to produce the original monomers, lactic acid and/or glycolic acid. Lactic and glycolic acids are nontoxic and eliminated safely via the Krebs cycle by conversion to carbon dioxide and water. The biocompatibility of PLA, PGA and PLGA polymers has been further examined in both non-ocular and ocular tissues of animals and humans. The findings indicate that the polymers are well tolerated.

Examples of PLA polymers, which may be utilized in an embodiment of the disclosure, include the RESOMER® product line available from Evonik Industries identified as, but are not limited to, R 207 S, R 202 S, R 202 H, R 203 S, R 203 H, R 205 S, R 208, R 206, and R 104. Examples of suitable PLA polymers include both acid terminated (H) and ester terminated (S) polymers with inherent viscosities ranging from approximately 0.15 to approximately 2.2 dL/g when measured at 0.1% w/v in CHCl₃ at 25° C. with an Ubbethode size 0c glass capillary viscometer.

In one embodiment, ester terminated (S) PLA polymers with an inherent viscosity ranging from approximately 0.25 to approximately 2.2 dL/g when measured at 0.1% w/v in CHCl₃ at 25° C. with an Ubbelhode size 0c glass capillary viscometer can be used in the present invention.

The synthesis of various molecular weights of PLA is possible. In one embodiment, PLA, such as RESOMER® R208, with an inherent viscosity of approximately 1.8 to approximately 2.2 dl/g (0.1% in chloroform, 25° C.), can be used. In another embodiment, PLA, such as RESOMER® R203S, with an inherent viscosity of approximately 0.25 to approximately 0.35 dl/g (0.1% in chloroform, 25° C.) can be used. In this embodiment, the R208 and R203S polymers can be ester end capped.

In one embodiment, the biodegradable matrix is comprised of a mixture of RESOMER® R208 and R203S polymers. In one such embodiment, R208 constitutes 67 +/- 5% of the biodegradable polymer matrix and R203S constitutes 33 +/- 5% of the biodegradable polymer matrix.

In some aspects, R203S comprises 21% ±10% and R208 comprises 44% ±10% and the API (e.g. travoprost) comprises 34% ±10% of the total intracameral implant.

Rcsomcr’s R203S and R208 are poly(D,L-lactide) or PLA ester-terminated polymers with the general structure (1):

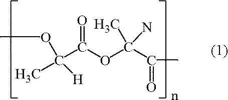

Examples of PLGA polymers, which may be utilized in an embodiment of the disclosure, include the RESOMER® Product line from Evonik Industries identified as, but are not limited to, RG 502, RG 502 H, RG 503, RG 503 H, RG 504, RG 504 11, RG 505, RG 506, RG 653 H, RG 752 H, RG 752 S, RG 753 H, RG 753 S, RG 755, RG 755 S, RG 756, RG 756 S, RG 757 S, RG 750 S, RG 858, and RG 858 S. Such PLGA polymers include both acid terminated (H) and ester terminated (S) polymers with inherent viscosities ranging from approximately 0.14 to approximately 1.7 dl/g when measured at 0.1% w/v in CHCl₃ at 25° C. with an Ubbelhode size 0c glass capillary viscometer. Example polymers used in various embodiments of the disclosure may include variation in the mole ratio of D,L-lactide to glycolide from approximately 50:50 to approximately 85:15, including, but not limited to, 50:50, 65:35, 75:25, and 85:15.

The synthesis of various molecular weights of PLGA with various D,L-lactide-glycolide ratios is possible. In one embodiment, PLGA, such as RESOMER® RG752S, with an inherent viscosity of approximately 0.16 to approximately 0.24 dl/g can be used. In one embodiment, PLGA, such as RESOMER® RG750S, with an inherent viscosity of approximately 0.8 to approximately 1.2 dl/g can be used. In one embodiment, PLGA, such as RESOMER® RG502S, with an inherent viscosity of approximately 0.16 to approximately 0.24 dl/g can be used.

Resomer RG752S is a poly(D,L-lactide-co-glycolide) or ester-terminated PLGA copolymer (lactide:glycolide ratio of 75:25) with the general structure (2):

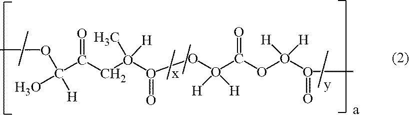

The polymers used to form the implants of the disclosure have independent properties associated with them that when combined provide the properties needed to provide sustained release of a therapeutically effective amount of a therapeutic agent.

A few of the primary polymer characteristics that control therapeutic agent release rates are the molecular weight distribution, polymer endgroup (i.e., acid or ester), and the ratio of polymers and/or copolymers in the polymer matrix. The present disclosure provides an example of a polymer matrix that possess desirable therapeutic agent release characteristics by manipulating one or more of the aforementioned properties to develop a suitable ocular implant.

The biodegradable polymeric materials which are included to form the implant’s polymeric matrix are often subject to enzymatic or hydrolytic instability Water soluble polymers may be cross-linked with hydrolytic or biodegradable unstable crosslinks to provide useful water insoluble polymers The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the implant is the relative average molecular weight of the polymeric composition employed in the implants. Different molecular weights of the same or different polymeric compositions may be included to modulate the release profile of the at least one therapeutic agent.

In an embodiment of the present disclosure, the polymers of the present implants are selected from biodegradable polymers, disclosed herein, that do not substantially swell when in the presence of the aqueous humor. By way of example but not limitation, PLGA polymers swell when used as the matrix material of drug delivery implants whereas PLA based polymer blends do not appreciably swell in the presence of the aqueous humor. Therefore, PLA polymer matrix materials are polymer matrix materials in embodiments of the present disclosure

### Drug Release Profile Manipulation

The rate of drug release from biodegradable implants depends on several factors. For example, the surface area of the implant, therapeutic agent content, and water solubility of the therapeutic agent, and speed of polymer degradation. For a homopolymer such as PLA, the drug release is also determined by (a) the lactide stereoisomeric composition (i.e., the amount of L- vs. D,L-lactide) and (b) molecular weight. Three additional factors that determine the degradation rate of PLGA copolymers are: (a) the lactide:glycolide ratio, (b) the lactide stereoisomeric composition (i.e., the amount of L- vs. DL-lactide), and (c) molecular weight.

The lactide:glycolide ratio and stereoisomeric composition are generally considered most important for PLGA degradation, as they determine polymer hydrophilicity and crystallinity. For instance, PLGA with a 1:1 ratio of lactic acid to glycolic acid degrades faster than PLA or PGA, and the degradation rate can be decreased by increasing the content of either lactide or glycolide. Polymers with degradation times ranging from weeks to years can be manufactured simply by customizing the lactide:glycolide ratio and lactide stereoisomeric composition.

The versatility of PGA, PLA, and PLGA allows for construction of delivery systems to tailor the drug release for treating a variety of ocular diseases.

When the versatility of PGA, PLA, and PLGA polymers are combined with the manufacturing techniques of the present disclosure, i.e. PRINT® technology (Envisia Therapeutics Inc ) particle fabrication, then a host of custom tailored and highly consistent and predictable drug release profiles can be created, which were not possible based upon the technology of the prior art, such as for example extrusion.

That is, with the present mold based particle fabrication technology, implants can be manufactured that exhibit a drug release profile that has highly reproducible characteristics from implant to implant. The drug release profiles exhibited by various implants of the present disclosure are consistent implant to implant and demonstrate variation that is not statistically significant. Consequently, the drug release profiles demonstrated by embodiments of the implants exhibit coefficients of variation that are within a confidence interval and does not impact the therapeutic delivery. The ability to produce implants that demonstrate such a high degree of consistent drug loading or release is an advancement over the state of the art.

### Drug Release Kinetics

Drug release from PLA- and PLGA-based polymer matrix drug delivery systems generally follows pseudo lirst-orcler or square root kinetics. A non-linear drug release profile from PLA- and PLGA-based polymer matrix drug delivery systems may also occur using polymeric matrices described herein.

Drug release is influenced by many factors including: polymer composition, therapeutic agent content, implant morphology, porosity, tortuosity, surface area, method of manufacture, and deviation from sink conditions, just to name a few. The present mold based manufacturing techniques-utilized in embodiments of the disclosure—are able to manipulate implant morphology, porosity, tortuosity, and surface area in ways that the prior art methods were incapable of doing. For instance, the highly consistent drug release profiles, highly consistent implant morphologies, and highly consistent homogeneous drug dispersions achievable by the present methods, were not available to prior art practitioners relegated to utilizing an extrusion based method of manufacture.

In general, therapeutic agent release occurs in 3 phases: (a) an initial burst release of therapeutic agent from the surface, (b) followed by a period of diffusional release, which is governed by the inherent dissolution of therapeutic agent (diffusion through internal pores into the surrounding media) and lastly, (c) therapeutic agent release associated with biodegradation of the polymer matrix. The rapid achievement of high therapeutic agent concentrations, followed by a longer period of continuous lower-dose release, makes such delivery systems ideally suited for acute-onset diseases that require a loading dose of therapeutic agent followed by tapering doses over a 1-day to 3-month period.

More recent advancements in PLGA-based drug delivery systems have allowed for biphasic release characteristics with an initial high (burst) rate of therapeutic agent release followed by substantially sustained zero-order (linear) kinetic release (i.e., therapeutic agent release rate from the polymer matrix is steady and independent of the therapeutic agent concentration in the surrounding milieu) over longer periods In addition, when desired for treating chronic diseases such as elevated IOP, these therapeutic agent delivery systems can be designed to have substantially steady state release following zero order kinetics from the onset.

Furthermore, recent advancements in PLA-based drug delivery systems have allowed for dynamic release profiles in which the release rate (and concentration) of the therapeutic agent fluctuates during degradation of the polymer matrix. Importantly, therapeutically relevant levels of the therapeutic agent and lowered IOP can be maintained with a dynamic release profile

### Therapeutic Agents

Suitable therapeutic agents for use in various embodiments of the disclosure may be found in the Orange Book published by the Food and Drug Administration, which lists therapeutic agents approved for treating ocular diseases including glaucoma and/or lowering IOP.

In some embodiments, the therapeutic agents that can be used according to the disclosure include: prostaglandins, prostaglandin prodrugs, prostaglandin analogues, prostamides, pharmaceutically acceptable salts thereof, and combinations thereof

Examples include prostaglandin receptor agonists, including prostaglandin E₁ (alprostadil), prostaglandin E₂ (dinoprostone), latanoprost, and travoprost. Latanoprost and travoprost are prostaglandin prodrugs (i.e. 1-isopropyl esters of a prostaglandin); however, they are referred to as prostaglandins, because they act on the prostaglandin F receptor, after being hydrolyzed to the 1-carboxylic acid.

A prostamide (also called a prostaglandin-ethanolamide) is pharmacologically unique from a prostaglandin (i.e. because prostamides act on a different cell receptor [the prostamide receptor] than do prostaglandins), and is a neutral lipid formed a as product of cyclo-oxygenase-2 (“COX-2”) enzyme oxygenation of an endocannabinoid (such as anandamide). Additionally, prostamides do not hydrolyze in situ to the 1-carboxylic acid. Examples of prostamides are bimatoprost (the synthetically made ethyl amide of 17-phenyl prostaglandin F₂ₐ) and prostamide F₂ₐ. Other prostaglandin analogues that can be used as therapeutic agents include, but are not limited to, unoprostone, and EP₂/EP₄ receptor agonists.

Prostaglandins as used herein also include one or more types of prostaglandin derivatives, prostaglandin analogues including prostamides and prostamide derivatives, prodrugs, salts thereof, and mixtures thereof

Suitable examples of the aforementioned drugs include, but are not limited to, latanoprost, travoprost, bimatoprost, tafluprost, and unoprostonc isopropyl.

In one embodiment, the disclosure utilizes travoprost, latanoprost, and bimatoprost. In another embodiment, the disclosure utilizes travoprost and latanoprost.

In a particular embodiment, the disclosure utilizes travoprost Travoprost has a molecular formula of C₂₆H₃₅F₃O₆ and a molecular weight of 500.548 g/mol.

The chemical structure (3) of travoprost is illustrated below:

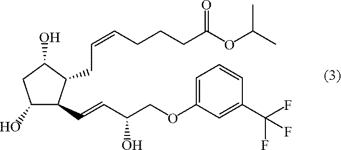 [SMILES: CC(C)OC(=O)CCC/C=C\C[C@@H]1[C@@H](/C=C/[C@@H](O)COc2cccc(C(F)(F)F)c2)[C@H](O)C[C@@H]1O]

### IUPAC Name: Propan-2-yl 7-[3,5-dihydroxy-2-[3-hydroxy-4-[3-(trifluoromethyl) phenoxy]-but-1-enyl]-cyclopentyt]hept-5-enoate

Travoprost, a prostaglandin analogue ester prodrug of the active moiety (+)-fluprostenol, is currently marketed as a 0.004% sterile, preserved, or preservative free, isotonic, multidose ophthalmic solution using well-known excipients. The formulations contain 40 ug of travoprost per mL of solution and is administered as a once a day drop with approximately 1 µg travoprost per day in patients with primary open-angle glaucoma or ocular hypertension to reduce intraocular pressure (TRAVATAN Z®, travoprost ophthalmic solution, Package Insert Alcon Laboratories, Inc. Fort Worth, TX 2004; and TRAVATAN®, travoprost ophthalmic solution, Package Insert. Alcon Laboratories, Inc. Fort Worth, TX 2013). Travoprost was first approved by the FDA as topical eye drops in 2001 under the tradename TRAVATAN® and more recently in 2006 under the tradename TRAVATAN Z®.

Travoprost is a synthetic prostaglandin analogue and is an isopropyl ester pro-drug of its free-acid active form, a selective and potent full agonist of the prostaglandin FP receptor with an EC₅₀ of 3.2 nM (Sharif NA, Kelly CR, Crider JY. “Agonist Activity of Bimatoprost, Travoprost, Latanoprost, Unoprostone Isopropyl Ester and Other Prostaglandin Analogs at the Cloned Human Ciliary Body FP Prostaglandin Receptor,” J Ocul Pharmacol Ther. 2002;18:313-324)

When dosed as topical eye drops, travoprost is hydrolyzed and appears in the aqueous humor as the free acid. Without being limited by theory, the mechanism of action by which travoprost lowers IOP is believed to occur by increasing the outflow of aqueous humor through the uveoscleral pathway, and possibly the trabecular meshwork. Lowering of IOP by travoprost has been studied in several animal models including monkey, dog, and cat (Gelatt KN, MacKay EO. “Effect of different dose schedules of travoprost on intraocular pressure and pupil size in the glaucomatous Beagle.” Vet Ophthalmol. 2004;7(1).53-57; and Bean GW. Camras CB. “Commercially available prostaglandin analogs for the reduction of intraocular pressure: similarities and differences,” Surv Ophthalmol. 2008;53 Suppl 1:S69-S84).

In ocular tissues, travoprost is known to rapidly hydrolyze to the free acid. Travoprost free acid is highly potent and selective for the FP receptor and is amongst the most potent in its classSee, Supra, Sharif et al.

A relative comparison of potency of parent and free acid for different members of the prostaglandin analogue class is presented in Table 1.

TABLE 1

Agonist Activity of Prostaglandin Analogues at the Cloned Human Ciliary Body FP Prostaglandin Receptor

| | |
|---|---|
| Compound | Functional Potency, EC₅₀ |
| Travoprost acid | EC₅₀ = 3.2 ± 0.6 nM |
| Bimatoprost acid | EC₅₀ = 5.8 ± 2.6 nM |
| Latanoprost acid | EC₅₀ = 54.6 + 12.4 nM |
| Travoprost | EC₅₀ = 42.3 ± 6.7 nM |
| Bimatoprost | EC₅₀ = 694 ± 293 nM |
| Latanoprost | EC₅₀ = 126 ± 34.2 nM |

### Pharmaceutical Compositions

In embodiments, the pharmaceutical composition is comprised of the biodegradable polymer matrix and at least one therapeutic agent.

The biodegradable polymer matrix is comprised of polymers meeting the desired characteristics. For example, desired characteristics may include a specific therapeutic agent release rate or a specific duration of action. The biodegradable polymer matrix may be comprised of one polymer, two polymers, or many polymers, such as three, four, five polymers, or more polymers.

In some embodiments, the compositions may comprise polymers utilizing the same monomer, such as compositions comprising various poly(D,L-lactide) homopolymers, or compositions comprising various poly(D),L-lactide-co-glycolide) copolymers. However, even if the polymers of the composition utilize the same monomer, the polymers may differ in other characteristics, such as, for example, inherent viscosity or mole ratio of D,L-lactide to glycolide.

In other embodiments, the compositions may comprise polymers utilizing different monomers, such as compositions comprising a poly(D, L-lactide-co-glycolide) copolymer and a poly(D,L-lactide) homopolymer. However, even if the polymers of the compositions utilize different monomers, the polymers may be similar in other characteristics, such as for example, inherent viscosity.

In one embodiment, the pharmaceutical composition comprises a biodegradable polymer matrix and at least one therapeutic agent homogeneously dispersed throughout the polymer matrix. For example, the polymer matrix contains a mixture of polymers comprising an ester end-capped biodegradable poly(D,L-lactide) homopolymer having an inherent viscosity at 25° C. in 0.1% w/v CHCl₃ of approximately 0.25 to approximately 0.35 dL/g and an ester end-capped biodegradable poly(D,L-lactide) homopolymer having an inherent viscosity at 25° C. in 0.1% w/v CHCl₃ of approximately 1.8 to approximately 2.2 dL/g. The ratio of the homopolymers in the polymer matrix can vary from approximately 15:85 to approximately 33:67 (lower inherent viscosity to higher inherent viscosity) Further, the presently discussed pharmaceutical composition comprising a biodegradable polymer matrix and at least one therapeutic agent, may, in certain embodiments, also exclude other polymers. That is, in some embodiments, the aforementioned polymer matrix only includes the two poly(D,L-lactide) homopolymers described above and no other polymer.

In an embodiment, the pharmaceutical composition comprises a biodegradable polymer matrix and at least one therapeutic agent homogeneously dispersed throughout the polymer matrix. For example, the polymer matrix contains a mixture of R203S and R208. The ratio of the homopolymers in the polymer matrix can vary from approximately 15:85 to approximately 33:67 (lower inherent viscosity to higher inherent viscosity). Further, the presently discussed pharmaceutical composition comprising a biodegradable polymer matrix and at least one therapeutic agent, may, in certain embodiments, also exclude other polymers. In an embodiment, the polymer matrix only includes R203S and R208 and excludes other polymers.

In one such embodiment, the biodegradable matrix includes a mixture of R203S and R208 polymers where the R203S polymer comprises 33% (± 1%, ±2%, ±5%, or ±10%) of the matrix and the R208 polymer comprises 67% (±1%, ±2%, ±5%, or ±10%) of the matrix.

In a further embodiment, the therapeutic agent comprises approximately 30-40% (±1%, ±2%, ±5%, or ±10%) of the total weight of the intracameral implant, and the remainder of the implant is composed of 20-30% (±1%, ±2%, ±5%, or ±10%) wt of the R203S polymer and 40-50% (±1%, ±2%, ±5%, or ±10%) wt R208 polymer.

In another embodiment, the intracameral implant comprises i) the active agent travoprost (33 +/- 1%, 2%, 5%, or 10% loading w/w); and ii) a biodegradable polymer matrix comprising: a poly(D,L-lactide) (PLA) blend of R203S (22 +/- 1%, 2%, 5%, or 10% w/w) and R208 (45 +/- 1%, 2%, 5%, or 10% w/w) polymers, wherein said ocular implant is molded from a mold cavity having dimensions of 225 µm × 225 µm × 2,925 µm.

In another embodiment, the ocular implant comprises: i) the active agent travoprost (34% +/- 1%, 2%, 5%, or 10% loading w/w); and ii) a biodegradable polymer matrix comprising: a poly(D,L-lactide) (PLA) blend of R203S (22% +/- 1%, 2%, 5%, or 10% w/w) and R208 (44% +/- 1%, 2%, 5%, or 10% w/w) polymers, wherein said ocular implant is molded from a mold cavity having dimensions of 150 µm × 150 µm × 1,500 µm.

In one embodiment, the pharmaceutical composition comprises a biodegradable polymer matrix and at least one therapeutic agent homogeneously dispersed throughout the polymer matrix For example, the polymer matrix contains a mixture of polymers comprising an ester end-capped biodegradable poly(D,L-lactide-co-glycolide) co-polymer having an inherent viscosity at 25° C. in 0.1% w/v CHCl₃ of approximately 0.8 to approximately 1.2 dL/g and an ester end-capped biodegradable poly(D,L-lactide) homopolymer having an inherent viscosity at 25° C. in 0.1% w/v CHCl₃ of approximately 1.8 to approximately 2.2 dL/g. The ratio of the polymers in the polymer matrix can vary from approximately 10:90 to approximately 20:80 (lower inherent viscosity to higher inherent viscosity). In embodiments, the ratio of the polymers in the polymer matrix is 15:80 (lower inherent viscosity to higher inherent viscosity). Further, the presently discussed pharmaceutical composition comprising a biodegradable polymer matrix and at least one therapeutic agent, may, in certain embodiments, also exclude other polymers. That is, in some embodiments, the aforementioned polymer matrix only includes the poly(D,L-lactide-co-glycolide) co-polymer and the poly(D,L-lactide) homopolymer described above and no other polymer.

In an embodiment, the pharmaceutical composition comprises a biodegradable polymer matrix and at least one therapeutic agent homogeneously dispersed throughout the polymer matrix. For example, the polymer matrix contains a mixture of RG750S and R208. The ratio of the polymers in the polymer matrix can vary from approximately 10:90 to approximately 20 80 (lower inherent viscosity to higher inherent viscosity). Further, the presently discussed pharmaceutical composition comprising a biodegradable polymer matrix and at least one therapeutic agent, may, in certain embodiments, also exclude other polymers. In an embodiment, the polymer matrix only includes RG750S and R208 and excludes other polymers.

In one such embodiment, the biodegradable matrix includes a mixture of RG750S and R208 polymers where the RG750S polymer comprises 15% (±1%, ±2%, ±5%, or ± 10%) of the matrix and the R208 polymer comprises 85% (±1%, ±2%, ±5%, or ± 10%) of the matrix.

In a further embodiment, the therapeutic agent comprises approximately 40-50% (±1%, ±2%, ±5%, or ±10%) of the total weight of the intracameral implant, and the remainder of the implant is composed of 5-10% (±1%, ±2%, ±5%, or ± 10%) wt of the RG750S polymer and 45-55% (±1%, ±2%, ±5%, or ±10%) wt R208 polymer.

In another embodiment, the intracameral implant comprises: i) the active agent travoprost (43 +/- 1%, 2%, 5%, or 10% loading w/w), and ii) a biodegradable polymer matrix comprising: a poly(D,L-lactide-co-glycolide) (PLGA) blend of RG750S (9 +/- 1%, 2%, 5%, or 10% w/w) and R208 (48 +/- 1%, 2%, 5%, or 10% w/w) polymers, wherein said ocular implant is molded from a mold cavity having dimensions of 210 µm × 200 µm × 1,500 µm.

In one embodiment, the polymer matrix contains a mixture of polymers comprising: (i) an ester end-capped biodegradable poly(D,L-lactide-co-glycolide) copolymer having an inherent viscosity at 25° C. in 0.1% w/v CHCl₃ of approximately 0.16 to approximately 0.24 dL/g, (ii) an ester end-capped biodegradable poly(D,L-lactide) homopolymer having an inherent viscosity at 25° C. in 0.1% w/v CIICl₃ of approximately 0.25 to approximately 0.35 dL/g, and (iii) an ester end-capped biodegradable poly(D,L-lactide) homopolymer having an inherent viscosity at 25° C. in 0.1% w/v CHCl₃ of approximately 1.8 to approximately 2.2 dL/g. The ratio of the homopolymers in the polymer matrix may be 10:67:23 (lower inherent viscosity to higher inherent viscosity). Further, the presently discussed pharmaceutical composition comprising a biodegradable polymer matrix and at least one therapeutic agent, may, in certain embodiments, also exclude other polymers. That is, in some embodiments, the aforementioned polymer matrix only includes the poly(D-L-lactide-co-glycolide) copolymer and the two poly(D,L-lactide) homopolymers described above and no other polymer

In an embodiment, the pharmaceutical composition comprises a biodegradable polymer matrix and at least one therapeutic agent homogeneously dispersed throughout the polymer matrix. For example, the polymer matrix contains a mixture of RG 502, R203S, and R208. The ratio of the polymers in the polymer matrix can vary from approximately 5:65:30 to approximately 10:70:20 (lower inherent viscosity to higher inherent viscosity). In embodiments, the ratio of the polymers in the polymer matrix is 10:67:23. Further, the presently discussed pharmaceutical composition comprising a biodegradable polymer matrix and at least one therapeutic agent, may, in certain embodiments, also exclude other polymers. In an embodiment, the polymer matrix only includes RG 502, R203S, and R208, and excludes other polymers.

In one such embodiment, the biodegradable matrix includes a mixture of RG 502, R203S, and R208 polymers, where the RG 502 polymer comprises 7% (±1%, ±2%, ±5%, or ±10%) of the matrix, the R203 comprises 45% (±1%, ±2%, ±5%, or ±10%), and the R208 polymer comprises 15% (±1%, ±2%, ±5%, or ±10%) of the matrix.

In a further embodiment, the therapeutic agent comprises approximately 30-40% (± 1%, ±2%, ±5%, or ± 10%) of the total weight of the intracameral implant, and the remainder of the implant is composed of 5-10% (±1%, ±2%, ±5%, or ± 10%) wt of the RG 502 polymer, 40-50% (± 1%, ±2%, ±5%, or ±10%) wt of the R203S polymer, and 10-20% (±1%, ±2%, ±5%, or ±10%) wt R208 polymer. In some aspects, the mold cavity utilized for creating an intracameral implant of the disclosure has dimensions of 225 µm (± 100 µm) × 225 µm (± 100 µm) × 2,925 µm (± 1000 µm); or 225 µm (± 50 µm) × 225 µm (± 50 µm) × 2,925 µm (± 500 µm); or 225 µm (+ 40 µm) × 225 µm (+ 40 µm) × 2,925 µm (+ 500 µm)

In some aspects, the mold cavity utilized for creating an intracameral implant of the disclosure has dimensions of 210 µm (± 100 µm) × 200 µm (± 100 µm) × 1,500 µm (± 1000 µm); or 210 µm (± 50 µm) × 200 µm (± 50 µm) × 1,500 µm (± 500 µm).

In some aspects, the mold cavity utilized for creating an intracamcral implant of the disclosure has dimensions of 150 µm (± 100 µm) × 150 µm (± 100 µm) × 1,500 µm (± 1000 µm); or 150 µm (± 50 µm) × 150 µm (± 50 µm) × 1,500 µm (± 500 µm); or 150 µm (± 40 µm) × 150 µm (± 40 µm) × 1,500 µm (± 500 µm).

In some aspects, the mold cavity utilized for creating an intracameral implant of the disclosure has dimensions of about 150 µm × 150 µm × 1,500 µm, but the implant that results from the PRINT™ processing procedure utilizing such a mold cavity has dimensions of about 190 µm × 130 µm × 1,500 µm, or about 130 µm × 190 µm × 1,500 µm.

In some aspects, the mold cavity utilized for creating an intracameral implant of the disclosure has dimensions of about 150 µm × 150 µm × 1,500 µm, but the implant that results from the PRINT™ processing procedure utilizing such a mold cavity has dimensions of about 190 µm (± 100 µm) × 130 µm (± 100 µm) × 1,500 µm (± 500 µm), or about 130 µm (± 100 µm) 190 µm (± 100 µm) × 1,500 µm (± 500 µm), or about 190 µm (± 50 µm) × 130 µm (± 50 µm) × 1,500 µm (± 100 µm); or about 130 µm (± 50 µm) × 190 µm (± 50 µm) × 1,500 µm (± 100 µm), or about 190 µm (± 40 µm) × 130 µm (± 40 µm) × 1,500 µm (± 100 µm); or about 130 µm (± 40 µm) × 190 µm (± 40 µm) × 1,500 µm (± 100 µm), or about 190 µm (± 30 µm) × 130 µm (± 30 µm) × 1,500 µm (± 100 µm); or about 130 µm (± 30 µm) × 190 µm (± 30 µm) × 1,500 µm (± 100 µm), or about 190 µm (± 20 µm) × 130 µm (± 20 µm) × 1,500 µm (± 100 µm), or about 130 µm (± 20 µm) × 190 µm (± 20 µm) × 1,500 µm (± 100 µm), or about 190 µm (± 10 µm) × 130 µm (± 10 µm) × 1,500 µm (± 100 µm), or about 130 µm (± 10 µm) × 190 µm (± 10 µm) × 1,500 µm (± 100 µm).

The aforementioned mold cavities used to fabricate the ocular implants may vary from the recited dimensions by ± 200 µm, ± 150 µm, ± 100 µm, ± 50 µm, ± 40 µm, ± 30 µm, ± 20 µm, ± 10 µm, or ± 5 µm, in various aspects. The aforementioned mold cavities used to fabricate the ocular implants may vary from the recited dimensions by less than or equal to about 50%, 40%, 30%, 20%, 10%, or 5% of any given dimension, in various aspects.

The aforementioned intracameral implants—which result from the discussed mold cavities used to fabricate the implants—may vary from the recited dimensions by ± 200 µm, ± 150 µm, ± 100 µm, ± 50 µm, ± 40 µm, ± 30 µm, ± 20 µm, ± 10 µm, or ± 5 µm, in various aspects. The aforementioned intracameral implants—which result from the discussed mold cavities used to fabricate the implants may vary from the recited dimensions by less than or equal to about 50%, 40%, 30%, 20%, 10%, or 5% of any given dimension, in various aspects The exact amount that the implant may vary from the utilized mold cavity will depend upon the particular PRINT™ processing parameters utilized to create the implant

In embodiments, the therapeutic agent is blended with the biodegradable polymer matrix to form the pharmaceutical composition. The amount of therapeutic agent used in the pharmaceutical composition depends on several factors such as: biodegradable polymer matrix selection, therapeutic agent selection, rate of release, duration of release desired, configuration of pharmaceutical composition, and ocular PK, to name a few

For example, the therapeutic agent content of the overall implant may comprise approximately 0.1 to approximately 60.0 weight percent of the total implants pharmaceutical composition. In some embodiments, the therapeutic agent comprises approximately 10.0 to approximately 50.0 weight percent of the pharmaceutical composition. In other embodiments, the therapeutic agent comprises approximately 20.0 to approximately 40.0 weight percent of the pharmaceutical composition In other embodiments, the therapeutic agent comprises approximately 30.0 to approximately 40.0 weight percent of the pharmaceutical composition. In yet other embodiments, the therapeutic agent comprises approximately 30.0 to approximately 35.0 weight percent of the pharmaceutical composition In yet still other embodiments, the therapeutic agent comprises approximately 30.0 weight percent of the pharmaceutical composition. Or in other embodiments the therapeutic agent comprises approximately 33.0 weight percent of the pharmaceutical composition. In still other embodiments the therapeutic agent comprises approximately 34.0 weight percent of the pharmaceutical composition

In embodiments, the pharmaceutical composition is prepared by dissolving the polymer or polymers and the therapeutic agent in a suitable solvent to create a homogeneous solution. For example, acetone, alcohol, acetonitrile, tetrahydrofuran, chloroform, and ethyl acetate may be used as solvents. Other solvents known in the art are also contemplated. The solvent is then allowed to evaporate, leaving behind a homogeneous film. The solution can be aseptically filtered prior to evaporation of the solvent.

### Fabrication of an Ocular Implant

Various methods may be used to produce the implants. Methods include, but are not limited to, solvent casting, phase separation, interfacial methods, molding, compression molding, injection molding, extrusion, co-extrusion, heat extrusion, die cutting, heat compression, and combinations thereof. In certain embodiments, the implants arc molded, preferably in polymeric molds.

In particular embodiments, the implants of the present disclosure are fabricated through the PRINT’ Technology (Liquidia Technologies, Inc.) particle fabrication. In particular, the implants are made by molding the materials intended to make up the implants in mold cavities.

The molds can be polymer-based molds and the mold cavities can be formed into any desired shape and dimension. Uniquely, as the implants are formed in the cavities of the mold, the implants are highly uniform with respect to shape, size, and composition. Due to the consistency among the physical and compositional makeup of each implant of the present pharmaceutical compositions, the pharmaceutical compositions of the present disclosure provide highly uniform release rates and dosing ranges The methods and materials for fabricating the implants of the present disclosure are further described and disclosed in the following issued patents and co-pending patent applications, each of which are incorporated herein by reference in their entirely. U.S. Pat. Nos. 8,518,316; 8,444,907, 8,420,124; 8,268,446; 8,263,129, 8,158,728; 8,128,393; 7,976,759, U.S. Pat. Application Publications Nos. 2014-0072632, 2014-0027948, 2013-0249138, 2013-0241107, 2013-0228950, 2013-0202729, 2013-0011618, 2013-0256354, 2012-0189728, 2010-0003291, 2009-0165320, 2008-0131692.

The mold cavities can be formed into various shapes and sizes. For example, the cavities may be shaped as a prism, rectangular prism, triangular prism, pyramid, square pyramid, triangular pyramid, cone, cylinder, torus, or rod. The cavities within a mold may have the same shape or may have different shapes. In certain aspects of the disclosure, the shapes of the implants are a cylinder, rectangular prism, or a rod. In a particular embodiment, the implant is a rod.

The mold cavities can be dimensioned from nanometer to micrometer to millimeter dimensions and larger. For certain embodiments of the disclosure, mold cavities are dimensioned in the micrometer and millimeter range. For example, cavities may have a smallest dimension of between approximately 50 nanometers and approximately 750 µm. In some aspects, the smallest mold cavity dimension may be between approximately 100 µm and approximately 300 µm. In other aspects, the smallest mold cavity dimension may be between approximately 125 µm and approximately 250 µm. The mold cavities may also have a largest dimension of between approximately 750 µm and approximately 10,000 µm. In other aspects, the largest mold cavity dimension may be between approximately 1,000 µm and approximately 5000 µm. In other aspects, the largest mold cavity dimension may be between approximately 1,000 µm and approximately 3,500 µm.

In one embodiment, a mold cavity having generally a rod shape with dimensions of 225 µm × 225 µm × 2,925 µm (W × H × L) is utilized to fabricate the implants of the present disclosure

In one embodiment, a mold cavity having generally a rod shape with dimensions of 215 µm × 230 µm × 2,925 µm (W × H × L) is utilized to fabricate the implants of the present disclosure.

In another embodiment, a mold cavity having generally a rod shape with dimensions of 150 µm × 150 µm × 1,500 µm (W × H × L) is used to fabricate the implants of the present disclosure.

In another embodiment, a mold cavity having generally a rod shape with dimensions of 210 µm × 200 µm × 1,550 µm (W × H × L) is used to fabricate the implants of the present disclosure.

In one embodiment, a mold cavity having generally a rod shape with dimensions of 175 µm × 215 µm × 1,390 µm (W × H × L) is utilized to fabricate the implants of the present disclosure.

Intracameral implants fabricated from the aforementioned mold cavities can vary from the recited dimensions of the mold cavity by about ± 500 µm, ± 400 µm, ± 300 µm, ± 200 µm, ± 100 µm, ± 90 µm, ± 80 µm, ± 70 µm, ± 60 µm, ± 50 µm, ± 40 µm, ± 30 µm, ± 20 µm, ± 10 µm, or ± 5 µm, in various aspects, including all values in between, or by about + 50%, or + 40%, or + 30%, or + 20%, or + 15%, or + 10%, or + 9%, or ± 8%, or ± 7%, or ± 6%, or ± 5%, or ± 4%, or ± 3%, or ± 2%, or ± 1%, in various aspects, including all values in between. For example, an intracameral implant can have dimensions that vary by about ± 5 µm to about ± 100 µm from the mold cavity with dimensions of 150 µm × 150 µm × 1,500 µm (W × H × L) used to fabricated the implant. Accordingly, in an embodiment, when using a mold cavity with dimensions of 150 µm 150 µm × 1,500 µm (W × H × L), the resultant implant can have dimensions of 190 µm × 130 µm × 1,500 µm, or 130 µm × 190 µm × 1,500 µm, or 190 µm × 130 µm × 1,420 µm, or 130 µm × 190 µm × 1,420 µm.

Once fabricated, the implants may remain on an array for storage, or may be harvested immediately for storage and/or utilization. Implants may be fabricated using sterile processes, or may be sterilized after fabrication. Thus, the present disclosure contemplates kits that include a storage array that has fabricated implants attached thereon. These storage array/implant kits provide a convenient method for mass shipping and distribution of the manufactured implants.

In other embodiments, the implants can be fabricated through the application of additive manufacturing techniques. Additive manufacturing, such as disclosed in U.S. Published Application US 2013/0295212 and the like can be utilized to either make the master template used in the PRINT process, utilized to make the mold used into the PRINT process otherwise disclosed herein or utilized to fabricate the implants directly.

In a particular embodiment, the implants are fabricated through the process of i) dissolving the polymer and active agent in a solvent, for example acetone, ii) casting the solution into a thin film; iii) drying the film; iv) folding the thin film onto itself; v) heating the folded thin film on a substrate to form a substrate; vi) positioning the thin film on the substrate onto a mold having mold cavities; vii) applying pressure, and in some embodiments heat, to the mold-thin film-substrate combination such that the thin film enters the mold cavities; ix) cooling; x) removing the substrate from the mold to provide implants that substantially mimic the size and shape of the mold cavities.

### Delivery Devices

In embodiments, a delivery device may be used to insert the implant into the eye or eyes for treatment of ocular diseases

Suitable devices can include a needle or needle-like applicator In some embodiments, the smallest dimension of an implant may range from approximately 50 µm to approximately 750 µm, and therefore a needle or needle-like applicator with a gauge ranging from approximately 22 to approximately 30 may be utilized. The delivery implant may be a syringe with an appropriately sized needle or may be a syringe-like implant with a needle-like applicator In an embodiment, the device uses a 27 gauge ultra thin wall needle. In aspects, the needle may have an inner diameter of 300 +/- 10 micrometers, or 250 +/- 10 micrometers, or 200 +/- 10 micrometers, or an inner diameter from about 300 to about 200 micrometers ± 10%. In aspects, a 27 gauge needle or smaller is utilized to deliver the intracameral implants, as it has been discovered that a 27 gauge or smaller needle will create a self healing wound.

Delivery routes include punctual, intravitreal, subconjunctival, lens, intrascleral, fornix, anterior sub-Tenon’s, suprachoroidal, posterior sub-Tenon’s, subretinal, anterior chamber, and posterior chamber, to name a few.

In embodiments, an implant or implants are delivered to the anterior chamber of a patient’s eye to treat glaucoma and/or elevated intraocular pressure.

### Kits

In embodiments, the implant and delivery device may be combined and presented as a kit for use.

The implant may be packaged separately from the delivery device and loaded into the delivery device just prior to use.

Alternatively, the implant may be loaded into the delivery implant prior to packaging. In this case, once the kit is opened, the delivery implant is ready for use.

Components may be sterilized individually and combined into a kit, or may be sterilized after being combined into a kit.

Further, as aforementioned, a kit may include an array with implants bound thereon.

### Use of Ocular Implant for Treatment

In one aspect of the disclosure, there is presented a method of treating glaucoma and/or elevated IOP. The method comprises placing a biodegiadable implant in an eye, degrading the implant, releasing a therapeutic agent which is effective to lower IOP, and thereby treating glaucoma and/or ocular hypertension.

In aspects of the disclosure, the eye is that of an animal. For example, a dog, cat, horse, cow (or any agricultural livestock), or human.

### Course of Treatment

Over the course of treatment, the biodegradable polymer matrix degrades releasing the therapeutic agent. Once the therapeutic agent has been completely released, the polymer matrix is expected to be gone. Complete polymer matrix degradation may take longer than the complete release of the therapeutic agent. Polymer matrix degradation may occur at the same rate as the release of the therapeutic agent.

Current treatments for glaucoma and/or elevated intraocular pressure require the patient to place drops in their eyes each day. The pharmaceutical composition of the disclosure is designed for sustained release of an effective amount of therapeutic agent, thus eliminating the need for daily drops.

For example, the pharmaceutical composition may be designed to release an effective amount of therapeutic agent for approximately one month, two months, three months, four months, five months, six months, seven months, eight months, nine months, ten months, eleven months, twelve months, or longer. In aspects, the pharmaceutical composition is designed to release an effective amount of therapeutic agent for one month, two months, three months, four months, five months, or six months. In other aspects, the pharmaceutical composition is designed to release an effective amount of therapeutic agent for three months, four months, five months, or six months. In aspects, the pharmaceutical composition releases therapeutic agent for longer than 6 months In aspects, the pharmaceutical composition releases therapeutic agent for a period of time between about 6 months and one year.

In an embodiment, the pharmaceutical composition is dosed in a repetitive manner. The dosing regimen provides a second dose of the pharmaceutical composition (i.e., implant) is dosed following the first implants release of its drug cargo. The dosing regimen also provides that a third dose of the pharmaceutical composition implants is not dosed until the polymer matrix of the implants of the second dosing are sufficiently degraded In an embodiment, the implant of the first dose fully degrade before the second dosing is administered In an embodiment, the repetitive dosing regimen can continue indefinitely.

The following non-limiting examples illustrate certain aspects of the present disclosure

### EXAMPLES

### Example 1: Preparation of Polymer Matrix/Therapeutic Agent Blends

The polymer matrix/therapeutic agent blend was prepared prior to fabrication of implants. Acetone was used to dissolve the polymers and therapeutic agent to create a homogeneous mixture. The polymer blend contained travoprost as the therapeutic agent. The resulting solution was aseptically filtered. After filtering, the acetone was evaporated leaving a thin film of homogeneous material. Table 2 details the composition of the various blends.

TABLE 2

Polymer Matrix/Therapeutic Agent Blend Ratios

| ID | Polymer Matrix Blend | R 208 (PLA) wt% | R 203 S (PLA) wt% | Travoprost wt% |
|---|---|---|---|---|
| 515-3 (also termed ENV515-3) | R203S/R208 33%/67% | 44.21 | 21.83 | 33.96 |
| 515-1 (also termed ENV515-1) | R203S/R208 33%/67% | 44.86 | 22.14 | 33.00 |

### Example 2: Fabrication of Molds

A mold of appropriate dimensions was created with the PRINT™ process. The mold had dimensions of 150 µm × 150 µm × 1,500 µm (ENV515-3) or 225 µm × 225 µm × 2,925 µm (ENV5I5-1).

### Example 3: Implant Fabrication via PRINT™

Implants were fabricated utilizing the polymer matrix/therapeutic agent blends of Example 1 and the molds of Example 2. Under aseptic conditions, a portion of polymer matrix/therapeutic agent blend was spread over a PET sheet and was heated for approximately 30 to 90 seconds until fluid. Once heated, the blend was covered with the mold of Example 2 which had the desired dimensions. Light pressure was applied using a roller to spread the blend over the mold area. The mold/blend laminate was then passed through a commercially available thermal laminator using the parameters in Table 3 below. The blend flowed into the mold cavities and assumed the shape of the mold cavities The blend was allowed to cool to room temperature and created individual implants in the mold cavities. The mold was then removed leaving a two-dimensional array of implants resting on the film. Individual implants were removed from the PET film utilizing forceps.

TABLE 3

Implant Fabrication Conditions

| Process Parameter | R 203 S / R 208 Matrix |
|---|---|
| Hot Plate Temperature, °C | 120-140 |
| Hot Plate Time, seconds | 30-90 |
| Laminator Temperature, °F | 320-360 |
| Laminator Speed, ft/min | 0.1-1.0 |
| Laminator Pressure, psi | 60-80 |
| Number of Passes | 5-8 |

### Example 4. Human Studies Using Intracameral Implants For Treatment Of Glaucoma

Aseptically produced, single-dose, intracameral implants comprised of a biodegradable polymer matrix and a prostaglandin analogue (travoprost) were designed to treat glaucoma in humans by lowering intraocular pressure. The prostaglandin analogue (travoprost) is released via hydrolysis of the polymer matrix, which delivers travoprost acid to the aqueous humor of a patient’s eye in a sustained manner. The biodegradable polymer matrix consists of a mixture of PLA polymers comprising a blend of R203S and R208.

The R203S polymer is an ester end capped biodegradable poly(D,L-to 0.35 dL/g measured at 0.1% w/v in CHCl₃ at 25° C. with a Ubbelhode size 0c glass capillary viscometer. The R208 polymer is an ester end capped biodegradable poly(D,L-lactidc) homopolymer having an inherent viscosity of 1.8 to 2.2 dL/g measured at 0.1% w/v in CHCl₃ at 25° C. with a Ubbelhode size 0c glass capillary viscometer. The ratio of R203S to R208 in the implants (when just considering the polymer matrix) is about 33% R203S to 67% R208, or about 30-40% R203S to about 60-70% R208. When considering the overall weight of the implant, the R203S is about 20-30% and the R208 is about 40-50% and the API is about 30-40%.

Each ENV515-3 implant included about 14.1 µg of travoprost. The percent composition of the intracamcral implant by weight (wt) is about 22% wt R203S, about 44% wt R208, and about 34% wt travoprost.

Each ENV515-1 implant included about 42.5 µg of travoprost. The percent composition of the intracameral implant by weight (wt) is about 22% wt R203S, about 45% wt R208, and about 33% wt travoprost.

Implants were fabricated using Particle Replication in Non-wetting Template (PRINT®) technology and rod-shaped mold cavities as described herein and further described and disclosed in the following patents and patent applications, each of which is incorporated herein by reference in their entirety: U.S. Pat. Nos 8,518,316; 8,444,907, 8,420,124; 8,268,446; 8,263,129, 8,158,728; 8,128,393, 7,976,759; U.S. Pat. Application Publications Nos. 2014-0072632, 2014-0027948, 2013-0249138, 2013-0241107, 2013-0228950, 2013-0202729, 2013-0011618, 2013-0256354, 2012-0189728, 2010-0003291, 2009-0165320, 2008-0131692.

The resultant ENV515-3 rod-shaped implants had dimensions of 190 × 130 × 1,500 µm (H × W × L) ± 10% of variation in each dimension. Accordingly, in some aspects, ENV515-3 rod-shaped implants had dimensions of about 180 × 132 × 1438 µm (H × W × L)

Implants were loaded into a single-use sterile applicator in a sterile field immediately prior to dosing and delivered directly into the anterior chamber of the patient’s eye via intracameral injection. Depending on the treatment arm to which a subject was assigned, two or three ENV515-3 implants were loaded into one eye of patient. The total dosage of travoprost for two ENV515-3 implants was 282 µg and for three ENV5 15-3 implants was 42.3 µg. The total dosage of travoprost for one ENV515-1 implant was 42.5 µg and for two ENV515-1 implants was 85.0 µg.

### Example 5. Experimental Design to Measure Iop in Patients With Glaucoma

The study was conducted as a multicenter, randomized, open-label parallel-group, dose-ranging, 28-day trial on subjects with bilateral open-angle glaucoma or ocular hypertension. Safety, tolerability, efficacy, aqueous humor PK, systemic exposure, and remaining travoprost in the implants at the two ENV515-3 and two ENV515-1 dose levels of the intracameral travoprost implants were assessed.

For the study, open-angle glaucoma was defined as focal non-full thickness rim thinning with no visual field (VF) changes or small isolated nasal step or paracentral scotoma or Seidel’s scotoma with visual field mean defect (MD) ≤ -8.0.

25 to 38 days before the study was initiated (referred to herein as the “washout period”), patients discontinued the use of all glaucoma mediations. IOP baseline was established 1 to 7 days before administration of the implant.

Five subjects received 2 implants in the study eye (2x ENV 515-3; total dose of 28.2 µg travoprost in the eye), and 11 subjects received 3 implants in the study eye (3x ENV 515-3; total dose of 42.3 µg travoprost in the eye).

Two subjects received 1 implant in the study eye (1x ENV 515-1; total dose of 42.5 µg travoprost in the eye), and 2 subjects received 2 implants in the study eye (2x ENV 515-1; total dose of 85.0 µg travoprost in the eye).

The non-study eye of each patient was dosed with TRAVATAN Z® (travoprost ophthalmic solution) 0.004% administered as indicated (1 eye drop per day at 8 p.m. ± 1 hour). After 4 weeks, implants were administered, and the implants were retrieved during cataract surgery.

The treatment arms of the study are summarized in scheme below.

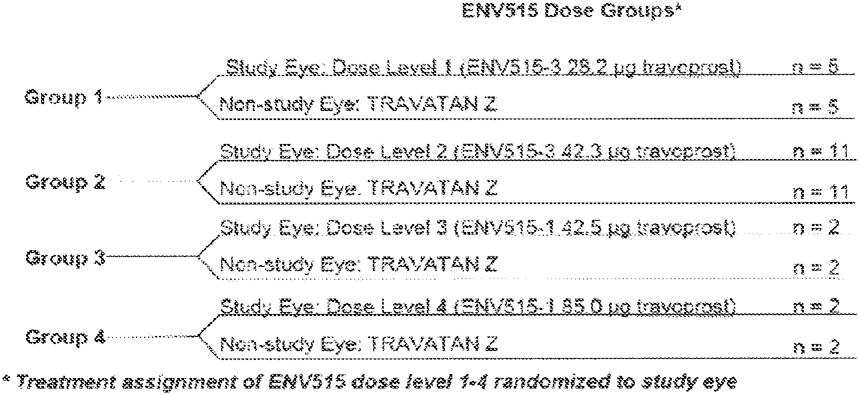

Diurnal IOP curves were measured at various points during the course of the study. (1) Initial IOP was measured at the start of the washout period after enrolment in the study, (2) Baseline IOP was measured prior to treatment (1 to 7 days before administration of the implant or TRAVATAN Z®); (3) Several IOP measurements were taken during the course of the 4 week study; and (4) Final IOP measurements were acquired 25 days after treatment was initiated. For the primary measurement of implant efficacy, the effect of the intracameral implant on IOP at Visit 8/Day 25 (±1 day) was analyzed in terms of % change in diurnal IOP from diurnal IOP baseline (established after the washout period).

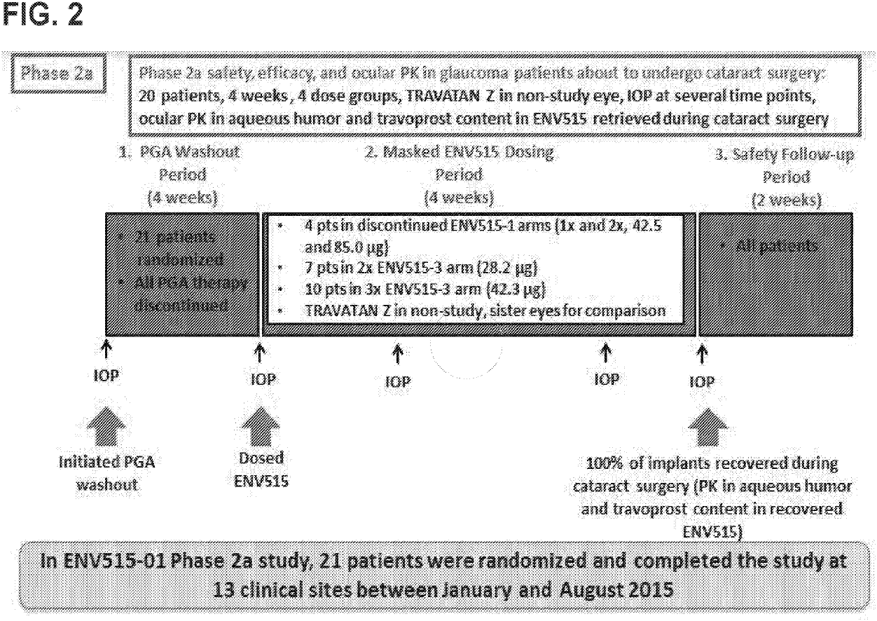

The design of the Phase 2a study is illustrated in FIG. 2.

### Example 6: Trial Objectives and Purpose

The primary objectives of the study were to: (1) Evaluate the safety and tolerability of ENV515 (travoprost) Intracameral Implants in subjects with bilateral ocular hypertension or early primary open-angle glaucoma; and (2) Evaluate the efficacy of ENV515 (travoprost) Intracameral Implants in lowering IOP in subjects with bilateral ocular hypertension or early primary open-angle glaucoma.

The secondary objectives of the study were to: (I) Determine the PK levels of travoprost in the aqueous humor at the time of the cataract surgery (4 weeks post implantation); (2) Determine the systemic exposure, i.e. the levels of travoprost in the plasma; and (3) Determine the residual level of travoprost in the implant removed at the time of the cataract surgery (4 weeks post implantation).

### Example 7 Selection of Patients

### Patient Eligibility Criteria

The following criteria were used to establish eligibility of individuals of either gender or any race to participate in the study:

1. Written informed consent provided prior to study procedures.

2. Subject was between 18 and 85 years of age.

3 Was willing to comply with the investigator’s instructions, attend study visits, and stop prior eye medications to treat glaucoma and/or ocular hypertension.

4. If female, subject was non-pregnant and non-lactating, and those of childbearing potential must be using an acceptable method of birth control (i.e., an intrauterine contraceptive device with a failure rate of <1%, hormonal contraceptives, or a barrier method). If a female subject was abstinent, she must agree to use one of the acceptable methods if she becomes sexually active.

5. Diagnosed with bilateral ocular hypertension or mild to moderate primary open-angle glaucoma and have open normal appearing anterior chamber angles (Shaffer classification Grade 3 or 4, angle of approach 20° or larger).

6. Was currently treated with topical PGA for ocular hypertension.

7. At the Baseline Visit after washout (Visit 2), IOP measurements satisfied the following criteria:
a. IOP at 8:00 a.m. (±30 minutes) and at 10:00 a.m. (±30 minutes) between 22-34 mm Hg in both eyes with a ≤4 mm Hg difference between the eyes; and
b. IOP at 4:00 p.m (±30 minutes) between 19-34 mm Hg in both eyes with a <4 mm Hg difference between each eye.

8. IOP ≤34 mm Hg in each eye at all other time points prior to the Baseline Visit (i e. Visit 2).

9. At the Screening Visit (Visit 1), the IOP in both eyes was at a level that was considered safe, so that clinical stability of vision and the optic nerve is likely throughout the trial.

10. Endothelial cell counts were at least 2000 cells/mm² and endothelial cell morphology at the Screening Visit (Visit 1) was normal as evaluated by central reading center.

11. Subject was a candidate for and had been scheduled for cataract extraction in a single eye within 60 days of Visit 1. Following cataract removal, the subject may have undergone additional procedures (e.g., iStent insertion).

Subjects meeting any one of the following conditions were excluded from the study:
1. Was currently diagnosed with closed angle glaucoma, exfoliation syndrome or exfoliation glaucoma, and pigment dispersion or secondary glaucoma;
2. Had a history of glaucoma-related surgery (trabeculectomy, cryotherapy, laser iridotomy, etc.);
3. Had intraocular conventional surgery, intraocular laser surgery, corneal refractive surgery or eyelids surgery within the past 3 months;
4. Was currently diagnosed with active infectious/noninfectious conjunctivitis, keratitis, uveitis, or moderate to severe blepharitis in either eye; (Chronic mild blepharitis or injection related to mild blepharitis, lid lag, mild dry eye or seasonal allergies are allowed.)
5. Was currently taking or had taken corticosteroids (oral, ocular, injectable, IV and/or topical) or used dermatology formulations of steroids in the vicinity of eyes in the 1 month prior to Visit 1 with the exception of inhaled, intranasal, or topical (dermal) steroids if on a stable dose; or had a history of chronic ocular corticosteroid (topical or intraocular) use within the past year,
6. Had a requirement for any ocular medications that were specifically disallowed in this protocol for any condition during the study or within the specified timeframe prior to Visit 2;
7. Had a history of recurrent corneal erosion syndrome, multiple corneal abrasions, or an abrasion that was slow to heal;
8. Had severe glaucoma with a mean defect (MD) worse than -8.0, central island of vision, or otherwise severe glaucoma that did not tolerate a possible short-term increase in intraocular pressure;
9. According to the investigator’s best judgment, were at risk for progression of glaucoma, visual field (VF) or visual acuity (VA) worsening as a consequence of participation in the trial;
10. Had any abnormality preventing reliable applanation tonometry in either eye;
11. Had any corneal opacity or are uncooperative in such a way that restricts adequate examination of the ocular fundus or anterior chamber in either eye,
12. Was unwilling to discontinue use of contact lenses at least 2 days prior to Visit 2 for soft lenses and at least 7 days prior to Visit 2 for rigid gas permeable (RGP) lenses through completion of the study at Visit 11;
13. Had progressive retinal or optic nerve disease apart from glaucoma;
14. Had any clinically significant, serious, or severe medical or psychiatric condition;
15. In the opinion of the investigator, was unable or unwilling to comply with study procedures, including attending the scheduled study visits;
16. Had a history, or a suspected history of drug or alcohol dependence in the preceding year;
17. Was unwilling to limit alcohol ingestion and smoking for the 8-hour period prior to and during study appointments after Visit 1;
18. Received any investigational drug within the past 30 days prior to Visit 1;
19. Had any history of alletgic hypersensitivity or poor tolerance to any components of the preparations used in this trial such as travoprost or PLA excipients;
20. Had a history of insufficient response to PGA topical treatment, i.e., are PGA nonresponders;
21. Was an employee of the clinical site that was directly involved in the management, administration, or support of this study or was an immediate family member of the same;
22. Had a central corneal thickness greater than 600 micrometers as determined by pachymetry at the Baseline Visit (Visit 2); or
23. Had prior intraocular surgery or any ocular or systemic condition that may confound the study outcome per the investigator’s recommendation.

At the randomized visit (Visit 3/ Day 1), an eligible subject must have continued to meet all of the inclusion/exclusion criteria defined above.

### Subject Withdrawal Criteria

Any subject who wished to withdraw from the study on his or her own accord for any reason was entitled to do so without obligation. Subjects who were withdrawn from the study prior to randomization were replaced. Any subject may have been removed from the study by the Investigator if it was deemed necessary for the subject’s safety.

In the event that withdrawal of a randomized subject was medically necessary or requested by the patient, the investigator made every attempt to complete all protocol safety assessments and visits through the cataract surgery combined with the removal of the ENV515 implants, and the post-surgery follow-up visits.

If subject withdrawal was required due to an adverse event (AE) or serious adverse event (SAE), the cataract surgery combined with the removal of the ENV515 implant(s) occurred as soon as possible based on the judgment of the Investigator and safety of the subject.

If an AE or SAE was unresolved at the time of the subject’s final study visit, an effort was made to follow the subject until the AE or SAE was resolved or stabilized (as defined), the subject was lost to follow-up, or there was some other resolution of the event. The investigator made every attempt to follow all SAEs to resolution.

### Specific Withdrawal Criteria

Following ongoing review of the data by the medical monitor, individual subject safety concerns were discussed between the medical monitor and investigator. If the investigator determined that a subject should have been discontinued and withdrawn from the study, the cataract surgery and removal of the ENV515 implants occurred as soon as possible based on the judgment of the investigator. Any rescue therapy or procedures were applied based on the judgment of the investigator

A subject may have been discontinued and withdrawn from the study at any time at the discretion of the investigator for any safety reason, including but not limited to those listed below:

1. JOP measurement of 35 mm Hg or greater in either eye at any measurement;

2. Any clinically significant pole changes, including but not limited to;
a) Cystoid macular edema (CME);
b) Retinal pigment epithelium (RPE); and
c) Disc rim pallor.

3. Pachymetry measurement of the central corneal thickness which revealed a change that falls outside of the normal variability when compared to the baseline measurement, such as:
a) An acute increase of 15% or greater in corneal thickness for a period of <24 hours after the instillation of study drug; or
b) A chronic increase of 10% or greater in corneal thickness for a period of >24 hours after the instillation of study drug.

4. A >10% decrease in central endothelial cell density as evaluated by the centralized reading center.

5. In the event that study discontinuation and withdrawal of a randomized subject was necessary, the investigator made every attempt to complete all protocol safety assessments and visits through the cataract surgery combined with the removal of the ENV515 implants, and the post-surgery follow-up visits. The cataract surgery combined with the removal of the ENV515 implants occurred as soon as possible based on the judgment of the investigator. Unless the Informed Consent was withdrawn, any subject was considered to be in the treatment phase of the study until the cataract surgery combined with ENV515 implant removal was completed, and such subjects continued to be followed and were expected to complete all pre- and post-surgery safety assessments and visits

### Example 8 Safety Evaluations

Following completion of the study, safety and tolerability assessments were to be conducted.

Endpoints for the study included;
1. Incidence of adverse events;
2. Changes in ophthalmic examination parameters (slit-lamp biomicroscopy, corneal staining, dilated funduscopic exam, anterior segment photos, pupil measurement, ocular symptom questionnaire, and visual field as measured by the Humphery Field Analyzer using program 24-2);
3. Changes in endothelial cell count and endothelial cell morphology using specular microscopy;
4. Changes in corneal thickness as measured using pachymetry;
5. Changes in IOP, including diurnal curve, as measured using a Goldman applanation tonometer,
6. Changes in visual acuity (VA) with manifest refraction using the Early Treatment of Diabetic Retinopathy Study (ETDRS) chart;
7. Changes in physical examination, vital signs, and laboratory parameters;
8. Rate of discontinuation form the study;
9. Drug levels (travoprost ester and travoprost free acid) in aqueous humor collected during cataract removal;
10. Drug levels (travoprost free acid) in plasma; and
11. Residual amount of travoprost (combined ester and free acid) remaining in the implants recovered during cataract surgery

### Example 9 Measurements and Evaluations

### Best-Corrected Distance Visual Acuity (Va) With Manifest Refraction Guidelines

VA was measured using the ETDRS chart VA was taken with the subject’s best- correction for distance at designated visits (method of correction was consistent across visits). Time was taken for careful refraction of subjects with reduced VA. Spectacle correction was not allowed. A consistent distance to the chart and method of measurement was used throughout the trial.

The VA was measured in the following way:

1. When performing VA, lighting was adjusted to approximate office levels, to present approximately uniform levels between the subject and the chart, and to be consistent throughout the trial.

2. For manifest refraction, “Chart R” was used. For actual vision testing, “Chart I” was used for the right eye and “Chart 2” for the left eye.

3. The technician asked the subject to read each letter slowly from the top of the chan and down as far as possible The technician did not point at letters to be read.

4. The subject was allowed no longer than 1 minute to see any 1 line. If the subject had difficulty reading a letter, they were encouraged to guess. When the subject could read no further, the technician asked the subject twice to read to the line below where the last correct letter was recognized When a letter was read correctly, the examiner recorded this on a score sheet with a layout identical to that of the chart.

5. The total number of letters missed at was recorded on the worksheet and entered in the case report form (CRF).

To record the Logarithm of the Minimum Angle of Resolution (LogMAR) visual acuity:

1. The last line from which at least 1 letter was read correctly was recorded; this is the Base LogMAR.

2. The total number of letters missed was recorded; this equals (N).

Example:
(a) 0.2 Base logMAR (the last line from which at least 1 letter was read correctly)
(b) 5 Total number of letters missed up to AND including the Base logMAR line

### Slit Lamp Biomicroscopy Exam Guidelines

Slit lamp biomicroscopy was performed using the investigator’s standard procedure This procedure was the same for all subjects observed at an investigator’s site. Observations were graded as normal or abnormal. In the event of abnormal observations, all findings were noted and specified as clinically significant or not clinically significant Hyperemia was evaluated against the provided scale and the findings were noted.

Observations for each eye were made of the following variables:
1. Eyelid
2. Conjunctiva
3. Cornea
4. Lens
5. Iris
6. Pupil
7. Eye motility
8. Anterior chamber

### Corneal Staining

The cornea was stained with non-preserved 2% fluorescein . When conducting all assessments, room temperature and humidity was relatively consistent throughout each visit and throughout the study, to the extent possible Observations were graded as normal or abnormal. In the event of abnormal observations, all findings were noted and specified as clinically significant or not clinically significant.

### Binocular Indirect Ophthalmoscopy (Dilated Fundus Exam) Guidelines

Dilated ophthalmoscopy was performed according to the investigator’s preferred procedure This procedure was the same for all subjects observed at an investigator’s site. Observations were graded as normal or abnormal. In the event of abnormal observations, all findings were noted and specified as clinically significant or not clinically significant The fundus was examined thoroughly and the following variables were examined:
1. Retina
2. Macula
3. Choroid
4. Vitreous
5. Optic nerve/disc
6. Cup/disc ratio

### Physical Examination

Physical examinations were performed excluding rectal, genitourinary, and breast examinations. The body systems evaluated were detailed in the source documents and CRF

### Vital Signs

Vital sign assessments included measurements of heart rate, blood pressure, and respiration rate

### Laboratory Safety Assessments and Systemic Exposure to Travoprost

Non-fasting laboratory samples were collected at Visits 1, 2, 6, and 10.

The following parameters were assessed as shown in Table 4 below:

TABLE 4

Parameters for Laboratory Safety Assessments and Systemic Exposure to Travoprost

| | | |
|---|---|---|
| Chemistry | Hematology | Urinalysis |
| ALT (SGPT) | Hematocrit | Color |
| AST (SOOT) | Hemoglobin | Specific gravity |
| Alkaline phosphatase | MCH | pH |
| Directbilubin | MCHC | Protein |
| Indirect bilirubin | MCV | Glucose |
| Total bilirubin | RBC | Ketones |
| GGT | WBC | Bilirubin |
| LDH | Basophils | Urobilinogen |
| Albumin | Eosinophils | Blood |
| Globulin | Lymphocytes | Nitrite |
| Total protein | Monoytes | Leukocyte esternse |
| Sodium | Neuirophils | Microscopic |
| Potassium | Platelets | |
| Calcium | RBC morphology | |
| Magnesium | | |
| Chloride | | |
| Bicarbonate | | |
| Phosphorous | | |
| Creatinine | | |
| Urea nitrogen | | |
| Uric acid | | |
| Creatine kinase | | |
| Glucose | | |
| Total cholesterol | | |
| Triglycerides | | |

Laboratory samples were additionally used to determine the systemic exposure to travoprost based on blood samples collected at Visits 6 and 10. A urine or serum pregnancy test was performed on all females of childbearing potential at Visit 1, 2, 3. and Visit 10.

### Anterior Chamber Optical Coherence Tomography (OCT)

Anterior chamber OCT images were acquired using a Zeiss Visantc (Car) Zeiss Meditec AG, Jena, Germany) or equivalent instrument. Images were acquired in the dark at the 6 o′clock position. Images were evaluated for angle opening distance at a central reading center. Additional details about collection, handling and interpretation of images were provided in the OCT manual

### Gonioscopy

Gonioscopy was performed to grade the iridocorneal angle according to the Shafter gonioscopy scale. Gonioscopy was also used to monitor the implant location. The Shaffer scale was used to describe the angle created between the plane of the iris and the cornea as follows:
Grade 4: 35 to 45 degrees, wide open, closure improbable
Grade 3: 20 to 35 degrees, moderately narrow, closure possible
Grade 2: <20 degrees, extremely narrow, closure probable
Grade 1: partly or totally closed, closure present

### Visual Field(Humphrey Program 24-2 SITA-Standard Strategy)

The VF assessment was performed on the Humphrey Field Analyzer using the program 24-2. All VF examinations were performed with the subject’s best correction for 33 cm. The pupil was at least 3 mm in diameter . If not, pharmacologic dilation was used for VF testing Central threshold was turned off Quantified single threshold perimetry was used if desired. Swedish Interactive Threshold Algorithms (ITA), Fastpac, or a similar program were used SITA Fast was not used

### Intraocular Pressure

All IOPs were measured with a Goldmann applanation tonometer. The calibration of the tonometer was checked at least monthly and recorded in a log. Diurnal curves were recorded at specified visits . The time of tonometry was recorded on the source document for all visits.

IOP was measured only after the biomicroscopic exam was completed and prior to pupil dilation. Measurements were taken by two qualified independent study site personnel using a Goldmann applanation tonometer affixed to a slit lamp with the subject seated. One person adjusted the dial in masked fashion and a second person read and recorded the value. The subject and slit lamp was adjusted so that the subject’s head was firmly positioned on the chin rest and against the forehead rest without leaning forward or straining. Both eyes were tested, with the right eye preceding the left eye Each IOP measurement was recorded

One person (“the measurer”) looked through the binocular viewer of the slit lamp at low power. The tension knob was pre-set at a low pressure value (4 to 6 mmHg) The measurer followed the image of the fluorescein-stained semicircles while he/she slowly rotated the tension knob until the inner borders of the fluorescein rings touched each other at the midpoint of their pulsarion in response to the cardiac cycle. When this image was reached, the measurer took his/her fingers off the tension knob and the second person (“the reader”) recorded the IOP reading along with the date and time of day in the source document, thus maintaining a masked IOP reading.

Three consecutive measurements were taken to determine IOP in the manner described above. All three measurements were recorded and the median IOP of the three measurements were recorded and used in the analysis.

### Pachymetry (Contact)

Following IOP measurements, the central corneal thickness of each eye was measured with the subject seated and visualizing fixation.

An ultrasonic pachymeter equipped with a solid tip probe was used

The probe tip was centered on the cornea and a measurement was taken once correctly positioned. 3 measurements were acquired (displayed in microns) for each eye and the values were averaged to obtain the corneal thickness measurement.

### Specular Microscopy (Non-Contact)

Assessment was performed according to the manufacturer’s specified instructions. The image analysis was conducted by centralized reading center.

### Pupil Measurement

Pupil diameter was measured in a room (not at the slit lamp) with standardized lighting that was used consistently the same way throughout the trial. The subject was instructed to gaze into the distance, and then the pupil diameter was compared to a standardized schematic. The same evaluator performed the measurement throughout the trial. A standardized schematic was provided by the sponsor.

### Aqueous Humor Sampling and Implant Recovery During Cataract Surgery

Cataract surgery and intraocular lens (IOL) implantation was conducted according to the discretion of the principal investigator per established protocols. Implant removal was conducted during the cataract surgery. The implant removal procedure described herein was used in nonclinical studies of ENV515. Based on observations in nonclinical studies of ENV515 in Beagle dogs, the implants retain their original size and shape, and do not disintegrate for at least 2 months in situ at the iridocorneal angle in vivo, and do not disintegrate when manipulated via instruments such as utrata forceps after 2 months in situ in vivo.

The following study-specific procedures were performed during the cataract surgery:

1. The implant location(s) were identified by gonioscopy exam conducted during pre-surgety assessments.

2. Following the creation of the initial incision in the clear cornea, -100 µL of aqueous humor was sampled from the anterior chamber via provided tuberculin syringe with 30 gauge needle.

3 After the removal of the aqueous humor sample, implants were recovered from the anterior chamber.

4. A stream of buffered saline solution (BSS) was directed to the iridocorneal angle location where the implants have been identified until implants were dislodged from the iridocorneal angle and floated in the anterior chamber. Utrata forceps or equivalent instrument were used to grasp the implant and remove the impant(s) through the incision in the clear cornea created for cataract removal and IOL implantation.

5. The aqueous humor samples and recovered implants were treated.

### Example 10 Phase 2a In Vivo Studies in Human Eye

In vivo studies were conducted with the ENV515.1 and ENV515-3 intracameral implants. Intraocular pressure and other parameters were evaluated at multiple visits throughout the study as described herein in detail

### Example 10a. Visit 1: Screening Evaluation (-35 To -28 Days Before Implantation)

At Visit 1, subjects were screened, and if eligible, enrolled into the study. Before any study specific assessments were performed, written informed consent was obtained from each subject During the visit, the procedures described below were performed.

### Screening Assessments

Assessments were conducted in the following order. Both eyes were evaluated at all ophthalmic assessments:
1. Obtained written informed consent
2. Obtained medical history, ocular history, and demographics (can be performed anytime during the site visit and does not need to follow the order as written)
3. Evaluated and recorded subject’s medication usage (including concomitant medications taken within the past 30 days) (can be performed anytime during the site visit and does not need to follow the order as written)
4. Assessed BCVA (ETDRS) with manifest refraction. If dilation was required to properly conduct BCVA (ETDRS) with manifest refraction, the assessment was performed after the slit lamp biomicroscopy and IOP measurement.
5. Performed pupil measurement
6. Performed slit lamp biomicroscopy
7. Performed corneal staining
8. Measured IOP
9. Performed gonioscopy
10. Performed pachymetry (contact)
11. Performed specular microscopy (non-contact). Non-contact specular microscopy was performed anytime during the clinic visit and did not need to follow the order as written.
12. Assessed visual field
13. Performed anterior chamber OCT
14. Performed dilated funduscopic exam
15. Performed physical examination (could be performed anytime during the site visit and did not need to follow the order as written)
16. Assessed vital signs (can be performed anytime during the site visit and does not need to follow the order as written)
17. Collected non-fasting blood and urine for clinical laboratory tests
18. If female of childbearing potential, performed urine or serum pregnancy test
19. Verified that subject met all applicable entry criteria
20. Queried patient about whether or not they have experienced symptoms suggesting an AE; AEs were documented

At the end of the examination, subjects were asked to discontinue their current glaucoma medication(s) in what is referred to herein as the “washout period.” The duration of the washout period for different types of topical glaucoma therapies is described in detail herein. The subject was asked to return for the baseline visit after 4 weeks. The washout period may have been extended up to 2 weeks, if it remained safe for the subject, to accommodate the subject’s or the investigator’s schedule.

### Subject Instructions

Before subjects left the clinic, they received an appointment for their next study visit and the following instructions:

1. Discontinue use of all eyedrop medications until the end of the study (if appropriate).

2 With your doctor’s approval, you may be able to use artificial tear eye drops.

3. Remember not to use alcohol or tobacco products within 8 hours of your next clinic visit.

4 At Visits 2 and 8, be prepared for a long clinic visit. You will be expected to have IOP measurements at 8:00am 10:00 a.m., and 4:00 p.m. You may leave the clinic after the 10:00 a.m. assessments with your doctor’s approval.

5. Call your study site if you have any problems.

6. Remember not to wear contact lenses 2 days for soft contact lenses and 7 days for RGP lenses prior to next visit

### Example 10b Visit 2: Baseline (-7 To -1 Days Before Implantation)

Subjects were queried about changes in medications and whether or not they had experienced symptoms suggesting an AE. AEs were documented.

### Baseline Assessments

Assessments were conducted in the following general order. Both eyes were evaluated at all ophthalmic assessments:
1. Assessed BCVA (ETDRS) with manifest refraction. If dilation was required to properly conduct DCVA (ETDRS) with manifest refraction, the assessment was performed after the slit lamp biomicroscopy and IOP measurement.
2. Performed pupil measurement
3. Performed slit lamp biomicroscopy
4. Performed corneal staining
5. Measured IOP at 8:00 a.m. (±30 minutes). IOP must be between 22-34 mm Hg in both eyes with a ≤4 mm Hg difference between each eye at 8:00 a m (±30 minutes).
6. Performed gonioscopy
7. Performed pachymetry (contact)
8. Performed specular microscopy (non-contact). Non-contact specular microscopy could be performed anytime during the site visit and did not need to follow the order as written.
9. Performed anterior chamber optical coherence tomography (OCT)
10. Performed physical examination (could be performed anytime during the site visit and did not need to follow the order as written)
11. Assessed vital signs (could be performed anytime during the site visit and did not need to follow the order as written)
12. Collected non-fasting blood and urine for clinical laboratory tests
13. If female of childbearing potential, performed urine or serum pregnancy test
14. Measured IOP at 10:00 a.m. (±30 minutes) . IOP must have been between 22-34 mm Hg in both eyes with a ≤4 mm Hg difference between each eye at 10:00 a.m. (±30 minutes)
15. Measured IOP at 4:00 p.m. (±30 minutes) IOP must have been between 19-34 mm Hg in both eyes with a ≤4 mm Hg difference between each eye at 4:00 p.m. (±30 minutes)
16. Performed dilated funduscopic examination
17. Verified that subject meets all entry criteria

Subjects were expected to remain in the clinic for the completion of all procedures (~8:00 a.m to ~5:00 p.m.) However, at the discretion of the investigator, subjects were permitted to leave the clinic after completing the 10:00 a.m. IOP measurement and return to the clinic before the 4:00 p.m. IOP measurement Any subject that left the clinic was instructed to return no later than 30 minutes prior to the 4:00 p.m. IOP measurement

### Subject Instructions

Before subjects left the clinic, they received an appointment for their next study visit and the Subject Instructions defined above.

### Example 10c. Visit 3: Randomization And Treatment (Day 1 - Date Of Implantation)

Subject was queried about changes in medications and whether or not they experienced symptoms suggesting an AE AEs were documented.

### Pre-Dose Assessments

Assessments were conducted in the following general order. Both eyes were evaluated at all ophthalmic assessments:
1. Assessed BCVA (ETDRS) with manifest refraction. If dilation was required to properly conduct BCVA (ETDRS) with manifest refraction the assessment was performed after the slit lamp biomicroscopy and IOP measurement.
2. Performed slit lamp biomicroscopy
3. Performed corneal staining
4. Measured IOP at 8:00 a.m. (±30 minutes)
5. Instilled one drop of VIGAMOX into the study eye
6. If female of childbearing potential, performed urine or serum pregnancy test
7. Verified that subject meets all entry criteria

### Randomization and Study Drug Administration

Subjects were assessed to ensure they still qualified to participate in the study based on the inclusion/exclusion criteria and randomization criteria previously described The study principal investigator administered the first and only dose of study medication into the pre-surgical study eye. The ENV515 experimental medication was delivered at 10:00 a m (±30 minutes) TRAVATAN Z® was administered into the non-study eye by the subject at 8 p.m. (±30 minutes).

At the randomization/treatment visit (Visit 3), the subject’s pre-surgical eye will be randomly assigned to 1 of the 4 dose levels of ENV515 and subjects will receive 1 to 3 ENV515 (travoprost) Intracameral Implant(s) into the pre-surgical eye via intracameral injection administered via the provided intracameral implant applicator. The site will receive randomization information based on randomization schedule following Visit 2 specifying which ENV515 formulation (ENV515-1 or ENV515-3) and how many implants to administer. The randomization code for this open-label study will be computer-generated prior to the study start. To randomize a subject (Visit 3), the investigator (or designee) will confirm in the electronic CRF that the subject remains qualified for the study. The eCRF will automatically assign the dose and number of implants that the subject should receive based on a prospectively prepared computer generated code list.

### Post-Dose Assessments

1. Performed slit lamp biomicroscopy
2. Dispensed TRAVATAN Z to the subject

Subjects were expected to remain in the clinic for the completion of all procedures (±8:00 a.m. to ~12:00 p.m.); however, at the discretion of the investigator, subjects were permitted to leave the clinic following the completion of the dosing.

### Subject Instructions

Before subjects left the clinic, they received an appointment for their next study visit and the following instructions:
1. Remember to use TRAVATAN Z® once daily in the evening (as close to 8:00 p.m. as you can) in the eye that is NOT having cataract surgery. DO NOT PUT TRAVATAN Z® in the eye that had the ENV515-3 implants. Continue using TRAVATAN Z® only through Day 24, one day prior to Visit 8 (Day 25 ± 1 day) Please remember to bring TRAVATAN Z® with you to Visit 8 (Day 25 ± 1 day) so that it can be collected from you
2. Continue to withhold (not use) all your other eyedrop medications until the end of the study (if appropriate). With your doctor’s approval, you may be able to use artificial tear eye drops.
3. Remember not to use alcohol or tobacco products within 8 hours of your next clinic visit.
4. Call your study site if you have any problems.
5. Please avoid physical activities associated with jarring physical motions, such as horseback riding, for the rest of the study.

### Example 10D. Visit 4: Treatment Period (Day 3 ± 1 Day Post Implantation)

Queried subject about changes in concomitant medications and whether or not they had experienced symptoms suggesting an AE AEs were documented.

Assessments were conducted in the following order. Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement.
2. Performed slit lamp biomicroscopy
3. Performed corneal staining
4. Measured IOP at 8:00 a.m. (±30 minutes)
5. Performed gonioscopy
6. Performed anterior chamber OCT

Subjects were expected to remain in the clinic for the completion of all procedures (~8.00 a.m. to ~10:00 a.m). Before subjects left the clinic, they received an appointment for their next study visit and the same Subject Instructions provided at Visit 3 as previously defined herein.

### Example 10E. Visit 5: Treatment Period (Day 7 ± 1 Day Post Implantation)

Subjects were queried about changes in concomitant medications and whether or not they have experienced symptoms suggesting an AE. AEs were documented.

Assessments were conducted in the following order. Both eyes were evaluated at all ophthalmic assessments:
1. Assessed BCVA (ETDRS) with manifest refraction If dilation was required to properly conduct BCVA (ETDRS) with manifest refraction the assessment was performed after the slit lamp biomicroscopy and IOP measurement.
2.. Performed slit lamp biomicroscopy
3. Performed corneal staining
4. Measured IOP at 8:00 a.m. (±30 minutes)

Subjects were expected to remain in the clinic for the completion of all procedures (- 8:00 a.m. to -9:00 a.m.). Before subjects left the clinic, they received an appointment for their next study visit and the same Subject instructions provided at Visit 3 as previously defined herein.

### Example 10F. Visit 6: Treatment Period (Day 14 ± 1 Day Post Implantation)

Queried subject about changes in concomitant medications and whether or not they had experienced symptoms suggesting an AE. AEs were documented.

Assessments were conducted in the following order. Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement.
2. Performed pupil measurement
3. Performed slit lamp biomicroscopy
4. Performed corneal staining
5. Measured IOP at 8:00 a.m. (±30 minutes)
6. Performed gonioscopy
7. Performed pachymetry (contact)
8. Performed specular microscopy (non-contact). Non-contact specular microscopy could be performed anytime during the site visit and did not need to follow the order as written.
9. Performed anterior chamber OCT
10. Performed dilated funduscopic exam
11. Collected non-fasting blood and urine for clinical laboratory tests and systemic PK

Subjects were expected to remain in the clinic for the completion of all procedures (-8:00 a.m. to ~ 10:00 am.). Before subjects left the clinic, they received an appointment for their next study visit and the same Subject Instructions provided at Visit 3 as previously defined herein.

### Example 10G. Visit 7: Treatment Period (Day 21 ± 1 Day Post Implantation)

Subject was queried about changes in concomitant medications and whether or not they have experienced symptoms suggesting an AE AEs were documented.

Assessments were conducted in the following order Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement.
2. Performed slit lamp biomicroscopy
3. Performed corneal staining
4. Measured IOP at 8:00 a.m. (±30 minutes)
5. Performed gonioscopy

Subjects were expected to remain in the clinic for the completion of all procedures (~8:00 a.m. to ~ 10:00 a.m.). Before subjects left the clinic, they received an appointment for their next study visit and the same Subject Instructions provided at Visit 3 as previously described herein.

### Example 10H. Visit 8: Treatment Period (Day 25 ± 1 Day Post Implantation)

Subject was queried about changes in concomitant medications and whether or not they had experienced symptoms suggesting an AE. AEs were documented. Collected TRAVATAN Z® from the subject

Assessments were conducted in the following order. Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement.
2. Performed pupil measurement
3. Performed slit lamp biomicroscopy
4. Performed corneal staining
5. Measured IOP at 8:00 a.m. (±30 minutes)
6. Performed gonioscopy
7. Performed pachymetry (contact)
8. Performed specular microscopy (non-contact). Non-contact specular microscopy could be performed anytime during the site visit and did not need to follow the order as written.
9. Performed anterior chamber OCT
10. Measured IOP at 10:00 a.m. (±30 minutes)
11. Measured IOP at 4:00 p.m. (±30 minutes)
12. Performed dilated funduscopic examination
13. Collected TRAVATAN Z® from the study subjects

Disbursement and First Administration of Pre-Surgical Medications

Following the completion of all assessments, the subjects received their pre-surgical anti-inflammatory and antibiotic medications: PRED FORTE®, PROLENSA®, and VIGAMOX®. The medications were administered by the subjects on Day 26, 27, and 28 twice a day for each medication, once in the morning and once in the evening. Following the removal of the ENV515 implant (Visit 9/Day 28), it was upon the discretion of the investigator to determine the post-operative medication regimen. Subjects were provided with instructions on use of these medications and what to do to prepare for their cataract surgery.

Subjects were expected to remain in the clinic for the completion of all procedures (-8:00 a.m. to ~4:30 p.m.). However, at the discretion of the investigator, subjects were permitted to leave the clinic after completing the 10:00 a.m. IOP measurement and returned to the clinic before the 4:00 p.m. IOP measurement. Any subject that left the clinic was instructed to return no later than 30 minutes prior to 4:00 p.m Before subjects left the clinic for the day, they received an appointment for their next study visit and the Subject Instructions previously described herein.

### Example 101. Visit 9: Cataract Surgery and Implant Removal (Day 28 Post Implantation)

Subject was queried about changes in concomitant medications and whether or not they had experienced symptoms suggesting an AE. AEs were documented.

### Pre-Sugery Assessment

Assessments were conducted in the following order. Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement
2. Performed slit lamp biomicroscopy
3. Performed corneal staining
4. Measured IOP at 8:00 a.m. (±30 minutes)
5. Performed gonioscopy to identify the location of the ENV515-3 implants to facilitate implant recovery during the cataract removal procedure
6. Additional assessment needed prior to cataract removal conducted per discretion of the principal investigate

### Cataract Removal Procedure Combined With Aqueous Humor Sampling and Implant Recovery

The cataract surgery and IOL implantation were conducted according to the discretion of the principal investigator per established protocols. The following study-specific procedures were performed during the cataract surgery:

The implant location(s) were identified by gonioscopy exam conducted during pre-surgery assessments.

Following the creation of the initial incision in the clear cornea, ~100 µL of aqueous humor was sampled from the anterior chamber via provided tuberculin syringe with 30 ga needle

After the removal of the aqueous humor sample, implants were recovered from the anterior chamber.

A stream of buffered saline solution (BSS) was directed to the iridocorneal angle location where the implant(s) have been identified until implant(s) were dislodged from the iridocorneal angle and floated in the anterior chamber. Utrata forceps or an equivalent instrument was used to grasp the implant(s) one at a time and remove the implant(s) through the incision in the clear cornea created for cataract removal and IOL implantation.

The aqueous humor samples and recovered implants were treated.

### Post-Surgical Assessments

The post-surgical assessments were conducted according to the discretion of the principal investigator per established protocols. Any observations associated with a standard cataract extraction followed by intraocular lens implantation as conducted by the principal investigator per established protocols, such as expected levels of aqueous cells or flare, were not recorded as AEs. Subjects were expected to remain in the clinic for the completion of all procedures (~8:00 a.m. to ~12:00 p.m.); however, at the discretion of the investigator, subjects were permitted to leave the clinic after completing all procedures and assessments. Before subjects left the clinic, they received an appointment for their next study visit and Subject Instructions as previously described herein. IOP lowering medications were prescribed per the judgement of the principal investigator at this time

### Example 10J Visit 10: Follow-Up (Day 33 To 38 Post Implantation)

Subjects were queried about changes in medications and whether or not they had experienced symptoms suggesting an AE. AEs were documented. Any observations associated with a standard cataract extraction followed by intraocular lens implantation as conducted by the principal investigator per established protocols, such as expected levels of aqueous cells or flare, were not recorded as AEs

Assessments were conducted in the following order. Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement.
2. Performed pupil measurement
3. Performed slit lamp biomicroscopy
4. Performed corneal staining
5. Measured IOP at 8:00 a.m. (±30 minutes)
6. Performed gonioscopy
7. Performed pachymetry (contact)
8. Assessed visual field
9. Performed anterior chamber OCT
10. Performed dilated funduscopic exam
11. Performed physical examination (could have been performed anytime during the site visit and did not need to follow the order as written)
12. Assessed vital signs (could have been performed anytime during the site visit and did not need to follow the order as written)
13. Collected non-fasting blood and urine for clinical laboratory tests and systemic PK
14. If female of childbearing potential, performed urine or serum pregnancy test

Subjects were expected to remain in the clinic for the completion of all procedures (~8:00 a.m. to ~10.00 a.m.). Before subjects left the clinic, they received an appointment for their next study visit and the Subject Instructions previously described herein.

### Example 10K: Study Exit (Day 42 to 49 Post Implantation)

Subjects were queried about changes in concomitant medications and whether or not they had experienced symptoms suggesting an AE AEs were documented. Any observations associated with a standard cataract extraction followed by intraocular lens implantation as conducted by the principal investigator per established protocols, such as expected levels of aqueous cells or flare, were not recorded as AEs.

Assessments were conducted in the following order. Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement.
2. Performed slit lamp biomicroscopy
3. Performed corneal staining
4. Measured IOP at 8:00 a.m. (±30 minutes)
5. Performed specular microscopy (non-contact). Non-contact specular microscopy was performed anytime during the site visit and did not need to follow the order as written.
6. Completed the exit form
7. Discharged the subject from the trial

Subjects were expected to remain in the clinic for the completion of all procedures (~8:00 a.m. to ~ 10:00 a.m.) The subject exited the trial barring any clinically significant, possibly related or related unresolved AEs.

### Example 10L Unscheduled Visits

Patients may have needed to be seen at other times than the scheduled study visits for additional safety assessments or to follow-up, as medically necessary, on changes in clinical status or to follow-up on clinical laboratory or other findings. If an additional study visit occurred, the date and nature of the visit was documented.

During unscheduled visits, subject was queried about changes in concomitant medications and whether or not they had experienced symptoms suggesting an AE. AEs were documented

Assessments were conducted in the following order Both eyes were evaluated at all ophthalmic assessments:
1. Assessed BCVA (ETDRS) with manifest refraction. If dilation was required to properly conduct BCVA (ETDRS) with manifest refraction, the assessment was performed after the slit lamp biomicroscopy and IOP measurement.
2. Performed slit lamp biomicroscopy
3. Measured IOP at 8:00 a.m. (±30 minutes) or when feasible
4. Performed additional assessments as deemed necessary per the investigator’s discretion

### Example 11 Stopping Rules

### Stopping Rules for a Patient

If necessary, a subject was discontinued and the subject was withdrawn at any time during the study at the discretion of the investigator for any sound safety reason including but not limited to occurrence of an AE or SAE.

In the event that study discontinuation of a randomized subject was necessary, the investigator made every attempt to complete all protocol safety assessments and visits through the cataract surgery combined with the removal of the ENV515 implants and the post-surgery follow-up visits. The cataract surgery combined with the removal of the ENV515 implants occurred as soon as possible based on the judgment of the investigator and safety of the subject. Unless the Informed Consent was withdrawn, any subject was considered to be in the treatment phase of the study until the cataract surgery combined with ENV515 implant removal, and such subjects continued to be followed and were expected to complete all pre- and post- surgery safety assessments and visits.

Any woman who became pregnant while participating in the study was expected to make every attempt to complete all protocol safety assessments and visits per the judgment of the principal investigator. If such an event occurred, information on the pregnancy and outcome was requested. The pregnancy was entered onto the CRF and recorded in the subject chart Safety concerns for the mother and the fetus were discussed between the medical monitor and investigator. The investigator determined whether to conduct the cataract surgery and remove of the ENV515 implant(s) and when to conduct such procedure while considering the safety of the mother and the fetus.

The investigator made every attempt to complete all safety assessments and continue such assessments per their judgment until cataract surgery and removal of the ENV515 implant were conducted or the fetus was delivered, whichever comes later, or beyond if necessary based on the judgment of the investigator.

### Stopping Rules for the Study

The medical monitor evaluated safety data on a weekly basis. Consultation with the principal investigators occurred as was appropriate. Assessment of safety and tolerability included, but was not limited to, AE reports, corneal thickness, endothelial cell morphology, endothelial cell counts, and slit lamp examination.

Provisions were made for stopping the study based on various events, for example, a significant number of AEs or SAEs

### Example 12 Treatment of Subjects

### Example 12A. Treatments to Be Administered

### Implantation of ENV 515 Into the Study-Eye

Treatment will consist of a single intracameral injection of ENV515 (travoprost) intracameral implant(s) into a pre-surgical eye that is scheduled for cataract removal. A single drop of TRAVATAN Z will be administered into the non-study eye as indicated daily from Visit 3 (Day 1) to Day 24, one day prior to Visit 8 (Day 25 ± 1 day). TRAVATAN Z will be collected from the subjects during Visit 8 (Day 25, ± 1 day)

At the randomization/treatment visit (Visit 3), subject’s pre-surgical eye will be randomly assigned to 1 of the dose levels of ENV515 and subjects will receive 1 to 3 ENV515 (travoprost) Intracameral Implant(s) into the pre-surgical eye via intracameral injection administered via the provided intracameral implant applicator. All investigators will be trained in implant loading, administration and retrieval by Envisia. The site will receive randomization information based on randomization schedule following Visit 2 specifying which ENV515 formulation (ENV515-1 or ENV515-3) and how many implants to administer. The randomization code for this open label study will be computer-generated prior to the study start. To randomize a subject (Visit 3), the investigator (or designee) will confirm in the electronic CRF that the subject remains qualified for the study. The eCRF will automatically assign the dose and number of implants that the subject should receive based on a prospectively prepared computer generated code list. The study treatment assignment of 1 of 4 dose levels of ENV515 to be administered will be determined by the randomization code. ENV515-1 and ENV515-3 (travoprost) Intracameral Implant(s) will be supplied in sterile glass vials with 1 implant per vial. The sterile implant applicator will be provided in a Tyvek® pouch. The packagings will be opened and the implant applicator and the implants will be placed into a sterile field. The implants will be loaded into the implant applicator by the principal investigator immediately prior to dosing. The implant size (ENV515-1 or ENV515-3) and the number of implants to load into the implant applicator will be determined based on the randomization code identifying 1 of 4 dose levels described previously. Additionally, the study eye will be administered topical antibiotic VIGAMOX following the completion of the pre-dose assessments and immediately before and after the ENV515 implant administration as described below. The following instructions were distributed with the ENV515 implants and implant applicator

### Opening Instructions

1. Use sterile technique in sterile field to open primary packaging for the applicator and ENV515 implants.

2. Open ENV515 Phase 2a Implant Applicator packaging and place the sterile ENV515 applicator into sterile field.

3. Do not open glass vial containing implants until ready to load into the applicator.

### Instructions for Loading the Implant Into the Applicator By the Principal Investigator

1. Load ENV515 implant(s) into the ENV515 Phase 2a implant applicator in a sterile field using sterile technique via insertion through the beveled needle end The number of ENV515 implants will be specified in the randomization code.

### Instructions for Administration by Principal Investigator

1. Treat patient’s ocular surface with topical aneasthetic (proparacaine 0.5% or equivalent).

2. Treat patient’s ocular surface, periocular skin, eyelid margins and eyelashes with povidone iodine and wait 2 minutes

3. Insert lid speculum.

4. Instill one drop of VIGAMOX® into the study eye.

5. Administer the implant(s) into the anterior chamber via intracameral injection through clear, peripheral cornea. The needle should be advanced parallel with the iris, ~1 mm anterior to the limbus with the patient sitting at the slit lamp, or with the patient supine under the operating scope.

6. Instill one drop of VIGAMOX® into the study eye

One implant applicator and 5 glass vials with one ENV515-1 or ENV51 5-3 implant per vial were packaged in an appropriately labeled carton The label on the package minimally contained the following information: (i) each package contains no less than 5 glass vials with either one ENV515-1 or ENV515-3 implant/vial and one ENV5I5 implant applicator; (ii) study ENV515-01; (iii) storage temperature: (iv) and “Caution. Limited by Federal (or United States) Law to Investigational use” An unmasked disclosure panel was displayed on the bottle label of the study medication and minimally contained the following information (i) ENV515-01; and (ii) name of product. The study medications were stored in a secure area with limited access to study personnel under refrigerated storage at approximately 2 to 8° C.

### Treatment of Non-Study Eye With Travatan Z

TRAVATAN Z® was provided for the non-study eye with its original packing, labeling, and instructions for use. A single drop of TRAVATAN Z® was administered into the non-study eye as indicated daily from Visit 3 (Day 1) to Day 24, one day prior to Visit 8 (Day 25 ± 1 day). TRAVATAN Z® was collected from the subjects during Visit 8 (Day 25, ± 1 day).

### Example 12B. Concomitant Medications

### Permitted Medications

Medications permitted included systemic medications with the exception of oral, ocular, or IV steroids. Only non-preserved artificial tears were allowed to be administered as an ocular treatment. Medications not specifically excluded were taken as necessary

All medications taken by a subject 30 days prior to Visit 1 through the end of the study were recorded in the eCRF and the subject’s medical chart. The generic name (if known, otherwise the trade name) of the drug, dose, route of administration, duration of treatment (including start and stop dates), frequency, and indication were recorded for each medication

Topical medications that were administered to all subjects as part of conducting safety assessments or routine procedures were not required to be recorded in the CRF. For example, topical medications used for the following are not required to be recorded in the CRF: (i) Dilating agents; (ii) Anesthesia; and (iii) Staining (i.e., fluorescein).

### Example 12C. Medications Not Permitted

During the Screening Visit, subjects were asked to discontinue their current glaucoma medication(s), if applicable, for the appropriate time period (Table 5). The subjects were asked to return for the Baseline Visit within 4 weeks Subjects were scheduled to return for an interim IOP check if their washout period was longer than 4 weeks. The washout period was extended up to 2 weeks (if medically safe) to accommodate the subject’s or investigator’s schedule. Subjects discontinued the use of any glaucoma medication(s) with the exception of study medication for the duration of the study.

TABLE 5

Washout Periods for Topical IOP-lowering Therapies

| | |
|---|---|
| Type of Topical IOP-lowering Therapy | Duration of Washout Period |
| Beta-adrenergic blockers | 4 weeks |
| α-adrenergic agonists | 4 weeks |
| Epinephrine-related medications | 4 weeks |
| Pilocarpine or carbonic anhydrase inhibitors | 7 days |
| Prostaglandin analogs | 4 weeks |

The use of corticosteroids (oral, ocular, injectable, or IV) was disallowed with the exception of inhaled, intranasal or topical (dermal) steroids if on a stable dose.

In the event that a subject required the initiation of one or more of these medications during the study, the investigator consulted with the sponsor regarding the proper action that should be taken.

### Example 12D. Drug Accountability

Study medication was not shipped to any investigational site until the site had fulfilled all requisite regulatory requirements Accountability of study drug (ENV515) and TRAVATAN Z® for the non-study eye was conducted by the sponsor’s monitor or designee. Accountability was ascertained by performing reconciliation between the amount of drug sent to the site, the amount used and the amount unused at the time of reconciliation.

Clinical trial materials were shipped to the investigational sites under sealed conditions. Study drug shipment records were verified by comparing the shipment inventory sheet to the actual quantity of drug received at the site Accurate records of receipt and disposition of the study drug (e.g., dates, quantity, subject number, dose dispensed, returned, etc.) were maintained by the investigator or his/her designee. Study drug was stored under refrigerated storage at approximately 2 to 8° C., with controlled access.

At the end of the study, all study materials, including used and unused study drug (ENV515 and TRAVATAN Z®) were returned to the sponsor (or designee) or destroyed under the direction of the same. The removed implants were retained. The study monitor or designee verified drug accountability. All drug accounting procedures were completed before the study was considered complete.

### Example 12E. Maintenance of Randomization

A randomization code for the subject assignment of dose levels of ENV515-1 and ENV515-3 was computer-generated by either the sponsor or its designee. Randomization team members worked independently of other team members. Study personnel, study subjects, and project teams at Envisia, the medical monitor, and the CRO involved in the study were unmasked to treatment assignments. To randomize a subject (Visit 3), the investigator (or designee) confirmed in the electronic CRF that the subject remained qualified for the study. The eCRF automatically assigned the dose and number of implants that the subject received based on a prospectively prepared computer generated code list.

In the event of a medical need, the investigator treated each subject as needed. The study design allowed for removal of the ENV515 intracameral implant by scheduling the subject for cataract surgery at an earlier date as determined by the medical need during which the ENV515 implant(s) were removed.

### Example 13. Assessment of Efficacy

Assessments of efficacy included: IOP measurements completed at all visits. A diurnal curve of IOP measurements was completed on Visit 2/Baseline and Visit 8/Treatment Day 25.

The IOP assessments and their timing are outlined as previously described herein.

### Example 14. Statistics

### Example 14A. Statistical Methods

The primary objective of this trial is to evaluate the safety and tolerability of 4 dose levels of ENV515 (travoprost) Intracameral Implant in subjects with bilateral ocular hypertension or early primary open angle glaucoma. Subjects will be evenly randomized (2 subjects per dose in the 2 ENV515-1 dose groups. 5 subjects per dose in the 2 implants/eye ENV515-3 dose group and 11 subjects per dose in the 3 implants/eye ENV515-3 dose group for a total of 20 subjects) to active treatment, with one study pre-surgical eye selected to receive study medication and the other non-study eye receiving TRAVATAN Z. All arms will be enrolled in parallel.

Assessment of safety and tolerability occurred on a weekly basis and included, but was not limited to, adverse event reports, corneal thickness, endothelial cell morphology, endothelial cell counts, and slit lamp examination.

Since this study was not powered to allow formal hypothesis testing of toxicity rates or efficacy between dose groups, any examination of treatment differences was exploratory in nature. For all analyses, subject-level covariates were summarized within each group by treatment (Table 7). Eye-level covariates were summarized for each cell in the final row of Table 8.

[symbol]

A detailed statistical analysis plan describing all analyses, tables, figures, and listings included in the final clinical report was specified prior to study start, given the unmasked nature of the study.

### Subject Disposition, Demographic and Background Characteristics

Baseline demographic characteristics such as age and gender and clinical characteristics including VA, IOP, gonioscopy, and corneal thickness were summarized using descriptive statistics. Baseline was defined as the last measurement prior to administration of the first dose of study drug.

### Analysis of Efficacy

The efficacy parameter measured in this study was IOP change from pre-dose baseline. Exploratory analyses comparing the change in IOP over time between treated study pre-surgical eyes and contralateral non-study TRAVATAN Z″ eyes were performed. Difterences in IOP change from baseline between dose groups were explored.

### Analysis of Safety

Safety endpoints included adverse events, corneal thickness, VA, endothelial cell count and morphology, slit lamp biomicroscopy exam findings, corneal staining, binocular indirect ophthalmoscopy, visual field assessment, anterior segment photos, pupil measurement, vital signs, clinical laboratory values, physical exam findings, and rate of discontinuation from the study Compliance with study drug administration was also collected.

AEs were coded using the Medical Dictionary for Regulatory Activities (MedDRA) and categorized by system organ class using preferred terms. Events were tabulated with respect to their intensity and relationship to the study drug. Changes in corneal thickness, VA, endothelial cell count and morphology, slit lamp biomicroscopy exam findings, binocular indirect ophthalmoscopy, visual field assessment, anterior segment photos, and pupil measurement were summarized and compared between treated study eyes and across study arms using descriptive statistics. Continuous clinical laboratory values were summarized using mean and standard deviation for reported and change from baseline values Categorical clinical laboratory values were summarized using shift tables displaying the frequencies of subjects with abnormal or normal results In addition, subject specific data listings were provided for all safety measurements

All SAEs and other significant events, including withdrawals due to AEs were individually summarized in the clinical study report.

### Other Analyses

Any analyses not described here were specified in a detailed statistical analysis plan prior to beginning any analysis of study data.

### Example 14B. Sample Size Estimation

Since this trial was primarily a dose-finding safely and tolerability study and the first study of ENV515 in subjects, sample size estimation was not performed. This study enrolled up to 4 arms of 2-11 subjects treated unilaterally.

The proposed number of subjects was typical for a Phase 1/2a clinical trial and was sufficient to assess the safety and tolerability of the study drug. Assuming that 5 subjects received pooled active drug within a cohort, the probability of failing to observe a toxicity was determined for various true underlying toxicity rates from the binomial distribution (Table 9). For example, for a true underlying toxicity rate of 30%_{,} the probability of failing to observe toxicity with 5 subjects was 0.17. for a true toxicity rate of 40%, the probability of failing to observe toxicity was 0.08.

TABLE 9

Toxicity Probabilities (n = 5)

| | | | | | | | | | |
|---|---|---|---|---|---|---|---|---|---|
| True Toxicity Rate (%) | 10% | 20% | 30% | 40% | 50% | 60% | 70% | 80% | 90% |
| Probability of Failing to Observe Toxicity | 0.59 | 0.33 | 0.17 | 0.08 | 0.03 | 0.01 | 0.002 | <0.001 | <0.001 |

Due to the study design and discontinuation criteria in the protocol, subjects who received the ENV515 dose of study treatment and discontinued from the study for any reason were not replaced.

### Example 14C. Level of Significance

All exploratory statistical tests were 2-sided and nominal significance was determined at the 0.05 level.

### Example 14D. Procedure for Accounting for Missing, Unused, or Spurious Data

Any missing, unused, or spurious data was noted in the final statistical report.

### Example 14E. Procedure for Reporting Deviations From the Statistical Plan

Any deviations from the statistical analysis plan were described and a justification was given in the final clinical study report.

### Example 14F. Subjects to Be Included in the Analysis

Efficacy analysis was performed for all subjects randomized, who received active study drug and completed at least one post-baseline IOP assessment (the intent-to-treat or ITT population). A subset of the efficacy analysis was repeated using data from those subjects who completed all study visits and achieved reasonable compliance with the study protocol (the Per Protocol population). AEs and other safety parameters were analyzed for all subjects receiving at least one dose of study medication in the study (Safety population).

The below Tables 10-13 summarize some of the parameters of the study population.

TABLE 10

Study Participant Demographics

| | ENV515-3 | | ENV515-1 | | All Groups |
|---|---|---|---|---|---|
| | Group 1 (low dose) n = 7 n (%) | Group 2 (high dose) n = 10 n (%) | Group 3 (low dose) n = 2 n (%) | Group 4 (high dose) n = 2 n (%) | All Groups n = 21 n (%) |
| Male | 2 (28.6) | 7 (70.0) | 0 (0) | 0 (0) | 9 (42.9) |
| Female | 5 (71.4) | 3 (30.0) | 2 (100.0) | 2 (100.0) | 12(57.1) |
| Age | | | | | |
| Mean (SD) | 74.3 (4.7) | 720(4.2) | 713 (7.1) | 62.5 (8.2) | 71.8 (5.6) |
| Race | | | | | |
| White | 7 (100.0) | 7 (70.0) | 2 (100.0) | 2 (100.0) | 18 (85.7) |
| Black or African American | 0 (0.0) | 2 (20.0) | 0 (0.0) | 0 (0.0) | 2 (9.5) |
| Native Hawaiian or Pacific Islander | 0 (0.0) | 1 (0.0) | 0 (0.0) | 0 (0.0) | 1 (4.8) |
| Baseline Weight (lbs) | | | | | |
| Mean (SD) | 172.0 (40.9) | 207.2 (32.3) | 176.5 (44.6) | 165.5 (29.0) | 188.6 (38.1) |
| Body Mass Index (kg/m*) | | | | | |
| Mean (SD) | 29.0 (6.7) | 30.5 (6.7) | 305 (4.4) | 30.0 (7.3) | 30.0 (5.2) |

TABLE 11

Study Disposition Table

| | ENV V515-3 | | ENV515-1 | | All Groups |
|---|---|---|---|---|---|
| | Group 1 (low dose) n(%) | Group 2 (high dose) n(%) | Group 3 (low dose) n (%) | Group 4 (high dose) n (%) | All Groups n (%) |
| Radomized | 7 (100) | 10 (100) | 2 (100) | 2 (100) | 21 (100) |
| Treated | 7 (100) | 10 (100) | 2 (100) | 2 (100) | 21 (100) |
| Analysis Populations | | | | | |
| Safety | 7 (100) | 10 (100) | 2 (100) | 2 (100) | 21 (100) |
| Intent-to-treat | 7 (100) | 10 (100) | 2 (100) | 2 (100) | 21 (100) |
| Per-protocol | 7 (100) | 10 (100) | 2 (100) | 2 (100) | 21 (100) |
| Study Completion | | | | | |
| Completed | 7 (100) | 10 (100) | 2 (100) | 2 (100) | 21 (100) |
| Discontinued | 0 (0) | 0 (0) | 0 (0) | 0 (0) | 0 (0) |

TABLE 12

Study Participant Eye Color

| | ENV515-3 | | ENV515-1 | | All Groups |
|---|---|---|---|---|---|
| | Group 1 (low dose) n = 7 n (%) | Group 2 (high dose) n = 10 n (%) | Group 3 (low dose) n = 2 n (%) | Group 4 (high dose) n = 2 n (%) | All Groups n = 21 n (%) |
| Eye Color | | | | | |
| Brown | 2 (28.6) | 4 (40.0) | 2 (100.0) | 1 (50.0) | 9 (42.9) |
| Blue | 1 (14.3) | 5 (50.0) | 0 (0) | 0 (0) | 6 (28.6) |
| Hazel | 3 (42.9) | 1 (10.0) | 0(0) | 1 (50.0) | 5 (23.8) |
| Green | 1 (14.3) | 0 (0) | 0 (0) | 0 (0) | 1 (4.8) |
| Grey | 0 (0) | 0 (0) | 0 (0) | 0 (0) | 0 (0) |
| Black | 0 (0) | 0 (0) | 0 (0) | 0 (0) | 0 (0) |
| Other | 0 (0) | 0 (0) | 0 (0) | 0 (0) | 0 (0) |

TABLE 13

Baseline IOP of Study Participants

| | ENV515-3 | | | | ENV515-1 | | | | All Groups | |
|---|---|---|---|---|---|---|---|---|---|---|
| | Group 1 (low dose) n = 7 n (%) | | Group 2 (high dose) n = 10 n (%) | | Group 3 (low dose) a = 2 n (%) | | Group 4 (high dose) n = 2 n (%) | | All Groups n = 21 n (%) | |
| | SE | NSE | SE | NSE | SE | NSE | SE | NSE | SE | NSE |
| Intraocular Pressure (mmHg) | | | | | | | | | | |
| Mean | 24.6 | 24.3 | 24.1 | 23.4 | 24.8 | 24.0 | 26.7 | 24.2 | 24.6 | 23.8 |
| SD | 2.4 | 2.6 | 2.3 | 2.5 | 3.1 | 0.5 | 1.4 | 2.1 | 2.3 | 2.3 |
| Median | 23.7 | 23.3 | 24.8 | 22.8 | 24.8 | 24.0 | 26.7 | 24.2 | 24.3 | 23.7 |
| Range - Min, Max | 22.0, 28.3 | 21.7, 28.0 | 21.3, 27.7 | 21.0, 28.0 | 22.7, 27.0 | 23.7, 24.3 | 25.7, 27.7 | 22.7, 25.7 | 21.3, 28.3 | 21.0, 28.0 |
| Visual Acuity (LogMAR) | | | | | | | | | | |
| Mean | 1.6 | 2.1 | 1.7 | 1.7 | 2.0 | 0.5 | 1.5 | 2.0 | 1.7 | 1.8 |
| SD | 1.5 | 1.6 | 1.8 | 1.5 | 2.8 | 0.7 | 0.7 | 0.0 | 1.6 | 1.4 |
| Shafer Score | | | | | | | | | | |
| 3 | 5 | 5 | 3 | 3 | 1 | 1 | 0 | 0 | 9 | 9 |
| | (71.4) | (71.4) | (30.0) | (30.0) | (50.0) | (50.0) | (0.0) | (0.0) | (42.9) | (42.9) |
| 4 | 2 | 2 | 7 | 7 | 1 | 1 | 2 | 2 | 12 | 12 |
| | (28.6) | (28.6) | (70.0) | (70.0) | (50.0) | (50.0) | (100) | (100) | (57.1) | (57.1) |

### Example 15. Interim Analyses From Phase 2a Studies With Travoprost Intracameral Implants (Env515-3 And Env515-1): Intraocular Pressure Measured At 8 Am Through Day 25

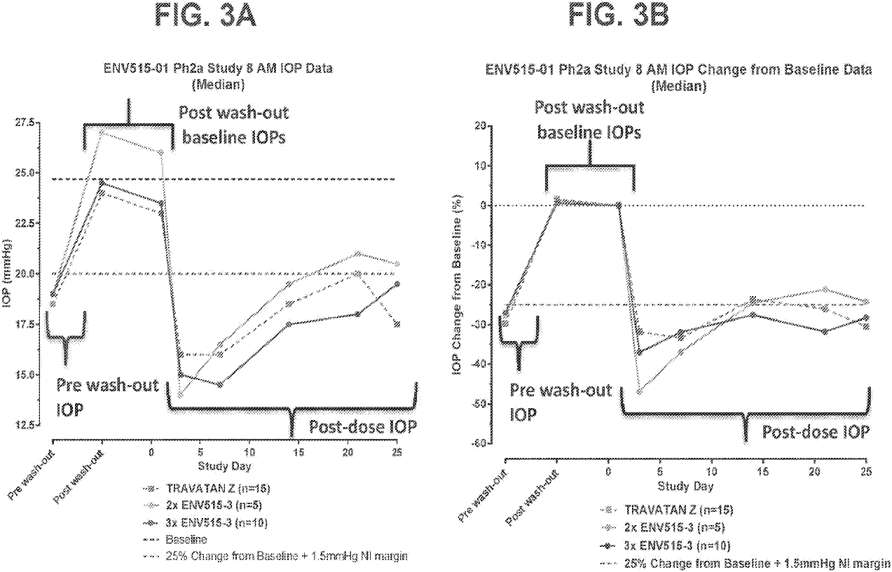

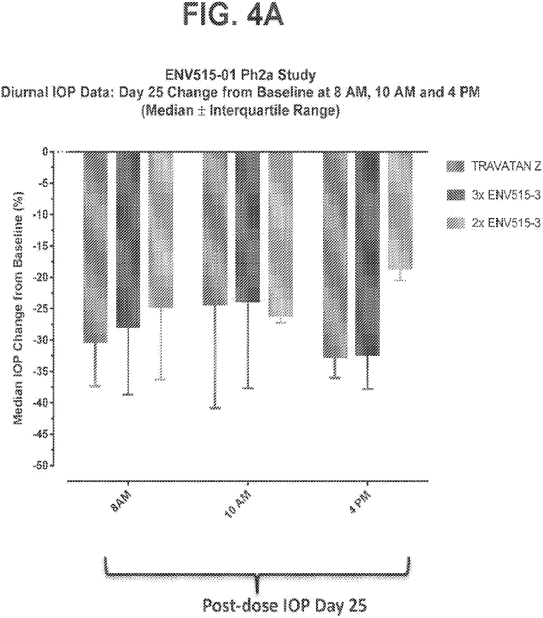

FIGS. 3A and 3B illustrate IOP measurements taken from the study eye (treated with 2-3 intracameral implants) and the non-study eye (treated with TRAVATAN Z®) over the course of the pre wash-out period, the post wash-out period, and 25 days of the phase 2a study. IOP measurements for subjects receiving 2 implants and subjects receiving 3 implants were averaged and plotted. For each time point (Study Day) displayed on the x-axis, the measured IOP (mm Hg) is displayed on the y-axis As shown in FIG. 3A, IOP was measured at the pre wash-out period, during the post wash-out period to establish a baseline of IOP without any mediation, and after treatment (i e IOP measured at days 0, 6, 10, 16, 20, and 26 after implantation). As shown in FIG. 3B, n post wash-out baseline was established by setting IOP measurements taken during washout period as 0.

FIGS. 3A and 3B show that ENV 515-3 Intracameral Implants were able to reduce IOP over 25 days by about 7.3 mm Hg or 29.3%. Both dosages of ENV 515-3 Intracameral Implants (2x ENV 515-3 and 3x ENV 515-3) significantly lowered IOP, with the higher dosage (3x ENV 515-3) showing a greater reduction of IOP

Reduction of IOP by ENV 515-3 Intracameral Implants was comparable to the reduction of IOP with daily treatment of TRAVATAN Z®.

### Example 16. Interim Analysis From Phase 2a Studies With Travaprost Intracameral Implant (Env 515-3): Diurnal IOP Change From Baseline On Day 25

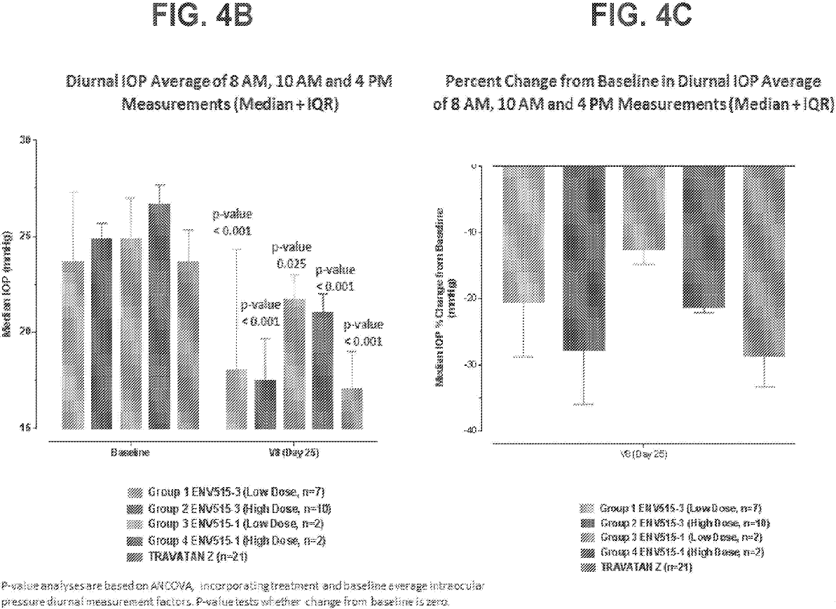

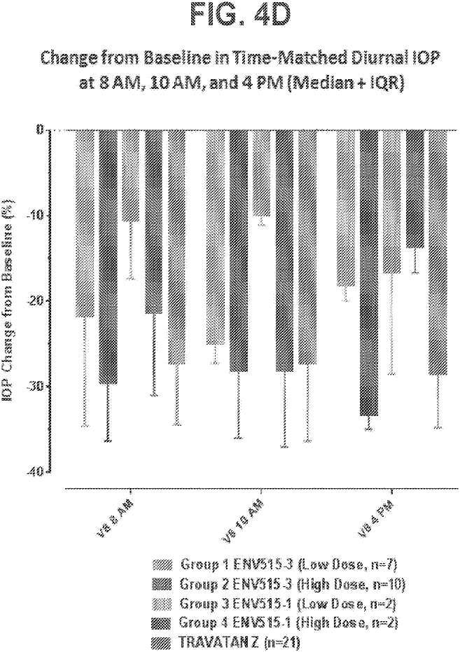

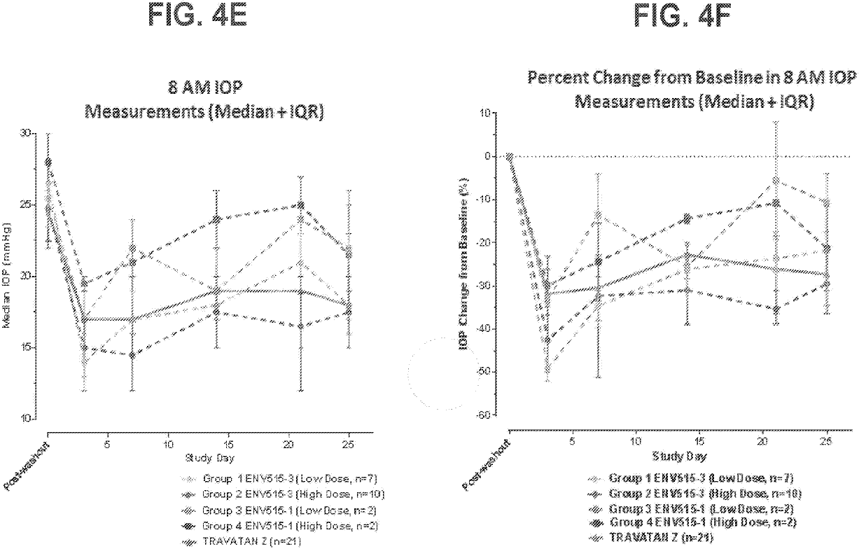

The results of diurnal IOP measurements on day 25 for the ENV 515-1 and 515-3 Intracameral Implants compared to TRAVATAN Z® are shown in FIGS. 4A-F.

For FIG. 4A, The percent change in IOP relative to the base line established at the post wash-out period is shown on the y-axis, and the x-axis shows the three time points (8 am, 10 am, and 4 pm) on day 25 at which diurnal IOP was measured.

The results indicate that ENV 515-3 Intracameral Implants lower IOP to a similar extent as TRAVATAN Z®, ENV 515-3, when administered as implants per eye (i.e. 3x ENV 515-3), lowered IOP as well as TRAVATAN Z®.

FIGS. 4B and 4C illustrate the average and percent change from baseline in Diurnal IOP Average (Average of 8 AM, 10 AM, and 4 PM IOPs), respectively.

FIG. 4D illustrates change from baseline in time-matched diurnal IOP at 8 AM, 10 AM and 4 PM.

FIGS. 4E and 4F illustrate the average 8 AM IOP and percent change from baseline in 8 AM IOP, respectively.

### Example 17. Sustained Release of Travoprost Via Env515-3 Intracameral Implant Lowers IOP at Concentrations Below Ec50 Calculated for Travatan Z® Eye Drops

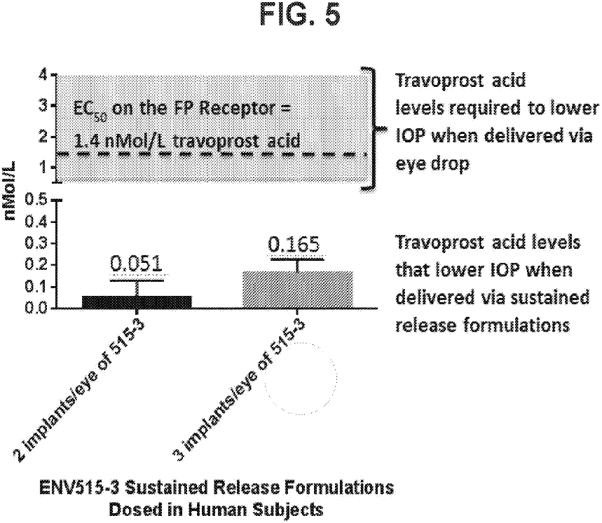

The concentration of free travoprost acid released from the Intracameral Implants into aqueous humor of the study eye was measured on day 28. FIG. 5 shows the concentration of free travoprost acid in the aqueous humor of the eye released from the ENV 515-3 Intraocular Implants.

Also shown in FIG. 5 is the EC₅₀ of travoprost acid, which is the concentration of travoprost acid that reduces IOP by half of the maximum IOP via binding the prostaglandin F (FP) receptor (i.e., the concentration of travoprost acid which induces a response halfway between the baseline and the maximum). The concentration of travoprost acid in the aqueous humor is provided on the y-axis. The x-axis shows the different treatments assessed to administer travoprost acid (i.e., 2x ENV 515-3, 3x ENV 515-3, and TRAVATAN Z®).

When two implants were administered into the study eye, the concentration of free travoprost acid in the aqueous humor was 0.051 nMol/L. When three implants were administered per eye, the concentration of free travoprost acid in the aqueous humor was 0.165 nMol/L.

For TRAVATAN Z®, the concentration of travoprost acid in the aqueous humor required to lower IOP ranged from about 0.8 nMol/L to about 4 nMol/L, as measured 1-3 hours after administration of an eye drop. See, Table 14.

The EC₅₀ measured for travoprost acid binding to the FP receptor is 1.4 nMol/L.

Thus, the results indicate that the sustained release of travoprost acid from the ENV 515-3 Intracameral Implants achieves a reduction in IOP at a significantly lower travoprost acid concentration than TRAVATAN Z® and significantly below the EC₅₀ value for the FP receptor. That is, ENV 515-3, when administered at 2 implants per eye, lowers IOP by delivering a travoprost acid concentration to the aqueous humor that is about 28 fold below the EC₅₀ for travoprost acid (i.e. 0.051 nMol/L for 2x ENV 515-3 compared to the EC₅₀ of 1.4 nMol/L). ENV 515-3, when administered as 3 implants per eye, lowers IOP by delivering a travoprost acid concentration to the aqueous humor that is about 8 fold below the EC₅₀ for travoprost acid (i.e. 0.165 nMol/L for 2x ENV 515-3 compared to the EC₅₀ of 1.4 nMol/L).

These results are summarized in Table 14 below.

TABLE 14

Sustained Release of Travoprost Acid from ENV 515-3 Intracameral Implant Lowers IOP at Significantly Lower Levels of Travoprost Acid in the Aqueous Humor

| | | | | | | |
|---|---|---|---|---|---|---|
| Prostaglandin Analog | Most potent measured EC₅₀ on FP receptor | Human aqueous humor levels 1-3 hours after travoprost eye drop administration | Human Aqueous Humor Levels 28 days after 2 ENV515-3 implants/eye were dosed with robust IOP- lowering | Human Aqueous Humor Levels 28 days after 3 ENV515-3 implants/eye were dosed with robust IOP-lowering | Ratio of EC50 levels vs. 2 implant/eye ENV515-3 levels in aqueous humor | Ratio of ECSO levels vs. 3 implant/eye ENV515-3 levels in aqueous humor |
| Travoprost acid | 1.4 nMol/L | 0.77 to 3.91 nMol/L | 0.051 nMol/L | 0.165 nMol/L | 28X | 8X |

### Example 18. Hyperemia Score Analysis Based on Standardized Hyperemia Scale

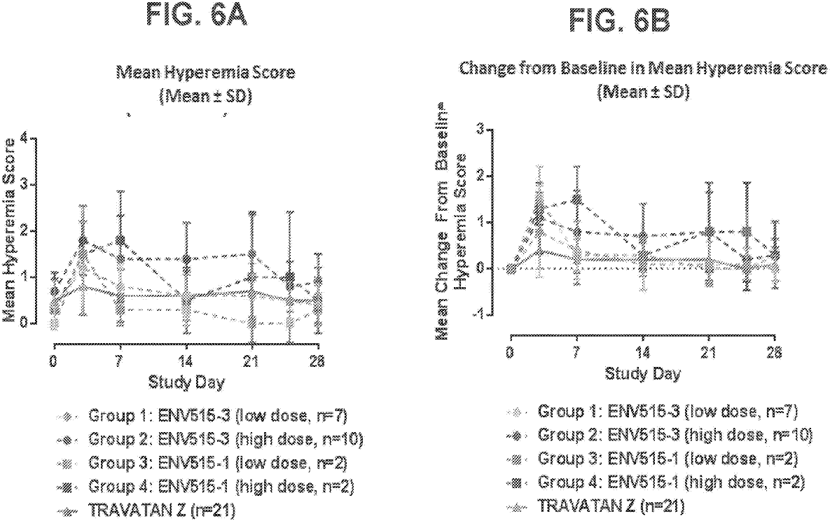

FIGS. 6A and 6B illustrate the mean hyperemia score and change from baseline in hyperemia score for study participants, respectively.

### Example 19. Aqueous Humor Travoprost Acid Levels Compared to Hyperemia

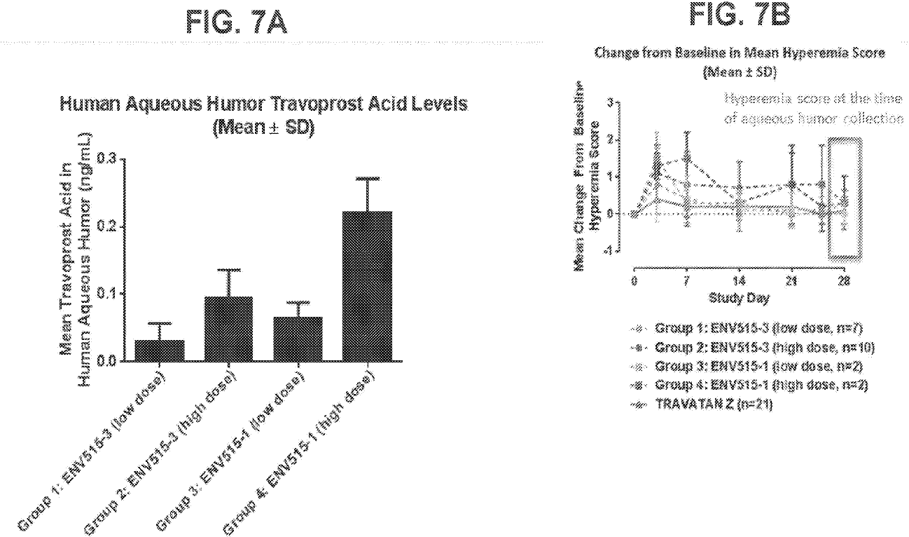

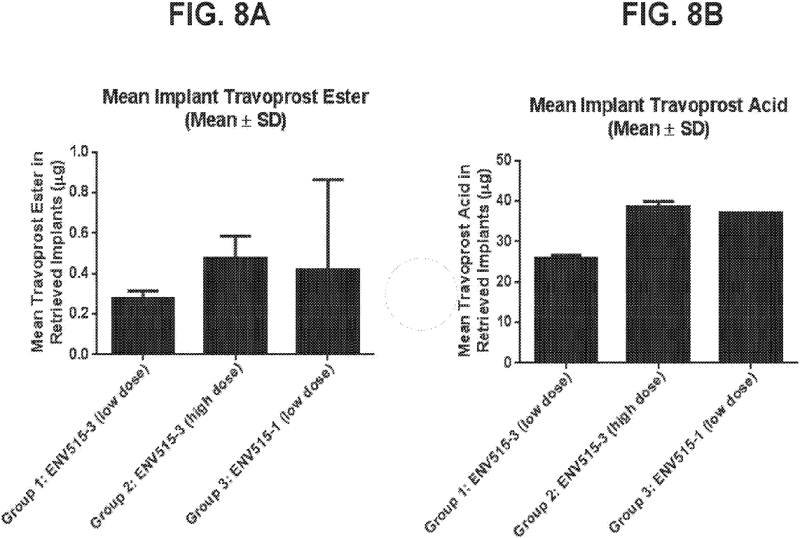

FIG. 7A illustrates the aqueous humor travoprost acid levels of study participants FIG. 7B illustrates mean hyperemia scores of study participants

As can be ascertained from these figures, the aqueous humor travoprost concentrations varied by an order of magnitude from low to high doses of ENV515 However, despite the large concentration difference, there was no apparent change in hyperemia.

### Example 20. Recovered Implant Analysis

FIG. 8A illustrates the mean recovered implant travoprost ester concentration. FIG. 8B illustrates the mean recovered implant travoprost acid concentration

The ability of the travoprost prodrug to convert to the active acid form within the intracameral implant, i.e. before being exuded into the eye, is remarkable and represents a previously unknown mechanism of delivering ester prodrugs to targeted locations within the eye.

Before the present study, it was assumed that the ester prodrug needed to be released into the aqueous humor before it could be converted to the acid form. However, surprisingly, the inventors have discovered that the unique attributes of the present intracameral implants allow the ester prodrug to be converted to the active acid form within the intracameral implant itself

### Conclusions From Human Studies of ENV515

The disclosure provides for newly identified, significantly lower levels of travoprost acid in aqueous humor sufficient for IOP lowering when achieved via sustained release formulations

The preferred levels of travoprost acid in aqueous humor, sufficient for IOP lowering when achieved via sustained release formulations of travoprost ester or travoprost acid (e.g. ENV515-3), are lower than the EC₅₀ of travoprost on the FP receptor of ~1.4 nMol/L.

The more preferred levels are lower than one half to one quarter of the EC₅₀ value or below ~0.17 nMol/L to ~ 0.05 nMol/L in aqueous humor.

Absolute value of the EC₅₀ depends on the methodology and model system used so both relative and absolute thresholds are provided.

Robust IOP-lowering effect in human subjects was demonstrated with travoprost acid values 8 to 28 times lower than EC₅₀ of travoprost acid (see FIG. 5 and Table 14) If new, more accurate EC₅₀ values are measured, the preferred levels of travoprost acid in aqueous humor would be relative to these new EC₅₀ values

There was no measurable travoprost in plasma in any subjects in the ENV515-01 Phase 2a study. Thus, the present study demonstrates that the ENV515 implants represent an improvement over the art

ENV515, dosed once on Day 1 in the 28-day dose-ranging Phase 2a study, achieved its primary efficacy endpoint, demonstrating statistically significant and clinically meaningful IOP-lowering effect at 25 days in change from baseline in mean diurnal IOP. The middle dose demonstrated numerically comparable treatment effect to topical TRAVATAN Z dosed in the non-study, fellow eye. The IOP-lowering treatment effect was sustained over the entire 25 days following a single dose of ENV515. The most common adverse event was early-onset transient hyperemia, or eye redness, related to the dosing procedure.

ENV515 is well tolerated at one dose level: ENV515-3 2 implants/eye. Larger ENV515-1 implants showed minor inferior transient corneal edema and small loss of endothelial cells. ENV515-3 implants dosed at 3 implants/eye also showed clinically significant endothelial cell loss. ENV515-3 at 2 and 3 implants per eye show sustained IOP reduction comparable to timolol and topical TRAVATAN Z, respectively. AH PK samples and retrieved implants validate long term release rate observed in dog & suggest longer duration in humans is likely. Implants easily and safely removed

### Example 21. Novel Design of the Env515-01 Phase 2as Cohort 1 Clinical Trial

Examples 5 to 20 included data generated using a novel clinical trial design displayed in FIG. 2. This design is particularly suitable for extended release formulations administered into the anterior chamber of the eye.

In established trial design of IOP lowering therapies formulated as extended release formulations and administered into the anterior chamber, glaucoma patients are administered an extended release formulation of IOP lowering agent and are studied over long periods of time. An example of such approach is demonstrated in the clinical studies of bimatoprost SR formulation (See, e.g., the study designs in NCT02250651 and NCT02247804, available at clinicaltrials.gov).

In this traditional trial design, no pharmacokinetic data is generated; if such formulations are not well tolerated, implants cannot be retrieved without subjecting patients to invasive surgical procedure, the rate of release of the drug from the formulation cannot be established; and such studies generally require prior extended toxicology studies and other non-clinical evaluations in animal models. No pharmacokinetic data can be established since the collection of aqueous humor and other relevant ocular tissues is invasive and creates anterior chamber inflammation and irritation for the glaucoma patients.

In contrast, a novel clinical trial design was employed for the first time in glaucoma patients to generate data described in Examples 5 to 20. This design employed a unique approach in utilizing glaucoma patients who were at the same time in need of cataract surgery (FIG. 2) The patients were dosed with ENV515 intracameral extended release therapy, and studied for safety and efficacy for 28 days. On Day 28, these patients underwent cataract removal followed by intraocular lens implantation to correct patients’ cataract. During this medically necessary procedure, aqueous humor was sampled and the ENV515 implants were removed without subjecting these patients to any additional surgical trauma beyond the cataract surgery. The aqueous humor was analyzed for content of travoprost released from ENV515 implants and the recovered implants were used to analyze true rate of drug release in situ in human patients’ anterior chamber of the eye (FIGS. 5, 7 and 8). This approach improved safety of the study for the enrolled patients: if there were any adverse events that required implant removal, patients could come in for their medically necessary cataract surgery at an earlier date and the ENV515 implants could be removed without subjecting the patients to any additional surgical trauma than was already needed due to the cataract formation and the medical need for its removal. Additionally, the human aqueous pharmacokinetic data and the true rate of drug release in the human eye enabled rapid evaluation of multiple formulations and projection of their duration of effect in human patients. Lastly, as the study duration was only 28 days for ENV515, which was designed as a therapy lasting longer than 6 months, this clinical trial required only a 28-day supporting toxicology evaluation in animal models.

### Example 22: Prophetic Example of Newly Identified, Significantly Lower Levels of Bimatoprost Acid in Aqueous Humor Sufficient For IOP Lowering When Achieved Via Sustained Release Formulations

The aforementioned novel study design, described in detail with respect to the ENV515 study, is expected to be used to generate the following results.

A 62 year old male presents with an intraocular pressure in his left eye of 30 mm Hg. Sustained release formulations of bimatoprost are inserted intracamerally: 50 µg dose of bimatoprost is administered via single administration of sustained release formulation on Day 1 of the study The patient’s intraocular pressure is monitored daily for one week, and then weekly thereafter through Day 28. On Day 28, patient undergoes cataract surgery in his left eye, during which the aqueous humor level is sampled and is analyzed for the levels of bimatoprost acid.

The patient’s IOP is expected to be lowered by 25% to 30% as an average IOP change from baseline on Days 1-28. The levels of bimatoprost acid, identified in patient’s aqueous humor that is collected on Day 28, are expected to be below EC₅₀ of bimatoprost acid on the FP receptor.

### Conclusions From Example 21

The disclosure provides for expected newly identified, significantly lower levels of bimatoprost acid in aqueous humor sufficient for IOP lowering when achieved via sustained release formulations

The expected preferred levels of bimatoprost acid in aqueous humor, sufficient for IOP lowering when achieved via sustained release formulations of bimatoprost prostamide or bimatoprost acid are anticipated to be lower than the EC₅₀ of bimatoprost acid on the FP receptor of ~3.3 nMol/L (see Table 15 for range of EC₅₀ potencies of bimatoprost acid and other PGAs on the FP receptor)

[symbol]

The expected more preferred levels are anticipated to be lower than one half to one quarter of the EC₅₀ value or below - 1.65 nMol/L to - 0.825 nMol/L in aqueous humor.

Absolute value of the EC₅₀ depends on the methodology and model system used so both relative and absolute thresholds are provided.

If new, more accurate EC₅₀ values are measured, the preferred levels of bimatoprost acid in aqueous humor would be relative to these new EC₅₀ values.

### Example 23. Clinically Significant Iop Lowering Sustained for at Least About 6 Months Following Implant Administration

Describe how experiment was conducted - human or dog studies, dosage, formulation (can reference earlier example showing formation.

Link data to table - Data showing IOP lowering for 6 months following administration of EVB515-3-1 is shown in FIG. X

Describe conclusions of data - i.e., The data indicates that ENV-515-3-1 can achieve clinically significant IOP lowering for 6 months or more

### Example 24: ENV515-01 Phase 2a Cohort 2

Cohort 2 is a 12-month study designed to assess the long-term safety, tolerability, effect on IOP, and systemic exposure of a single travoprost dose of 28.2 µg achieved via 2 ENV515-3 implants. The Cohort 2 phase of the study was conducted as a prospective, open-label, fellow-eye active-comparator controlled, multi-center 12-month trial in approximately 10 subjects with bilateral open-angle glaucoma or ocular hypertension In the Cohort 2 phase of the study, ENV515-3 implants were administered unilaterally in the study eye and followed for 12 months.

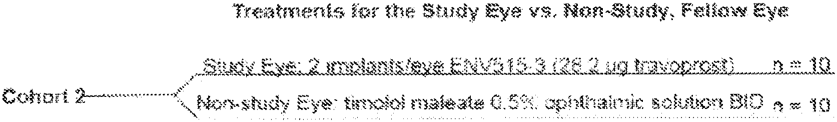

### Example 25: Clinically Significant Iop Lowering Sustained For at Least About 6 Months Following Implant Administration in Env515-01 Phase 2a Cohort 2 Clinical Trial (Examples 23 To Example 27)

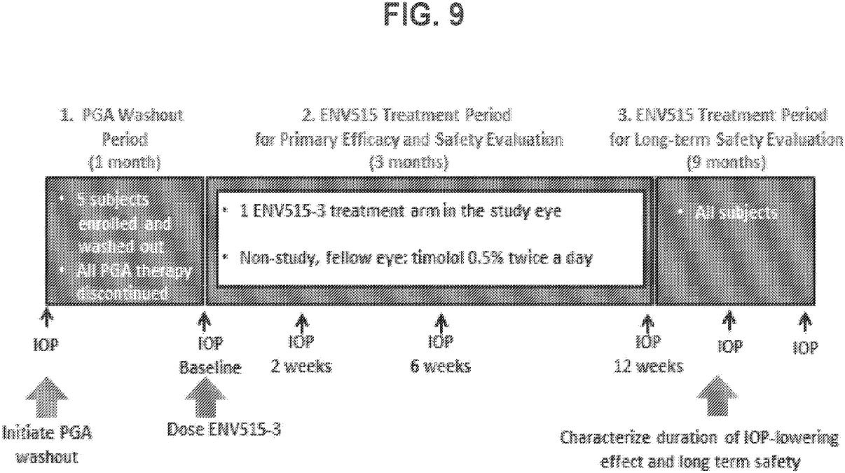

ENV515-01 Phase 2a Cohort 2 clinical trial was carried in glaucoma patients out as described in the clinical study protocol based on the design displayed in FIG. 9. Glaucoma patient disposition is presented in Table . Two ENV515-3 implants per eye were administered (14.1 ug/implant and 28.2 ug/eye) into the study eye via intracameral injection. A total of 5 patients were enrolled into the study across 2 sites. All patients completed the first 6 months of the study and there were no early discontinuations

TABLE 16

Patient Disposition Table for Cohort 2 of ENV515-01 Phase 2a Study

| | ENV515-3 (n=5) |
|---|---|
| Category | n (%) |
| Treated | 5 (100) |
| Safety | 5 (100) |
| Intent-to-treat | 5 (100) |
| Per-protocol | 4 (100) |
| Completed 3 months post dose | 5 (100) |
| Discontinued | 0 (0) |

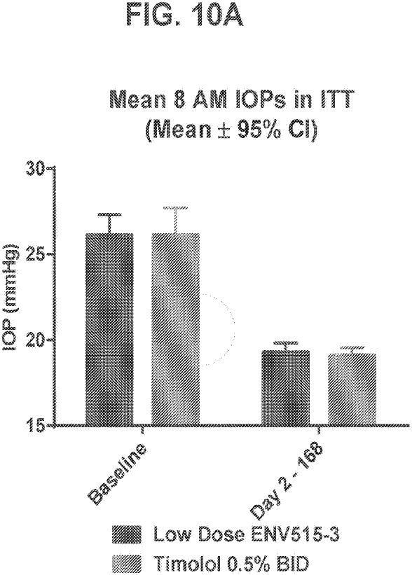

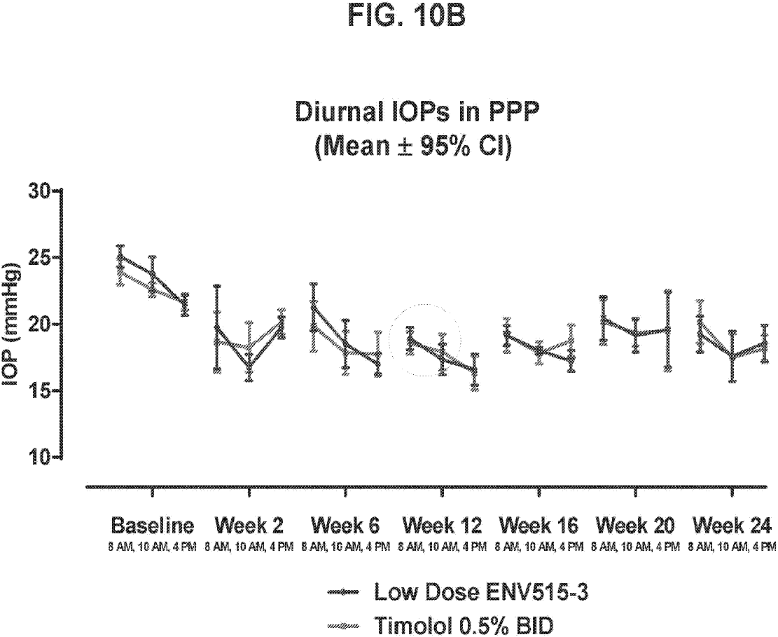

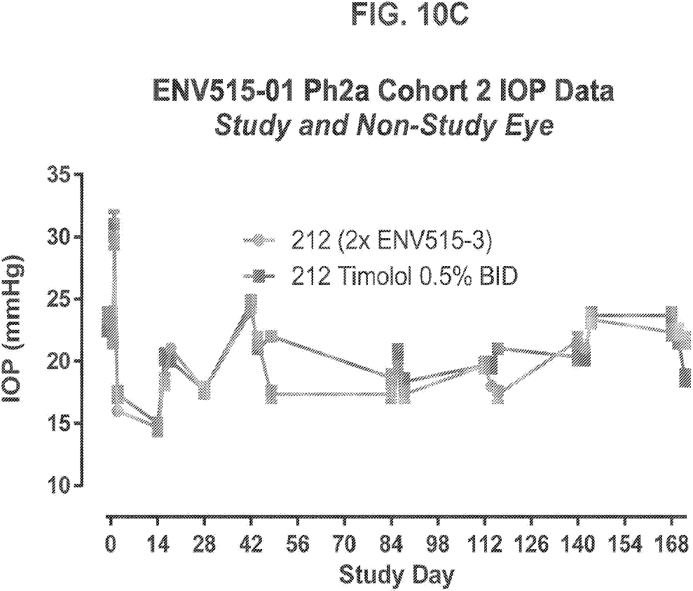

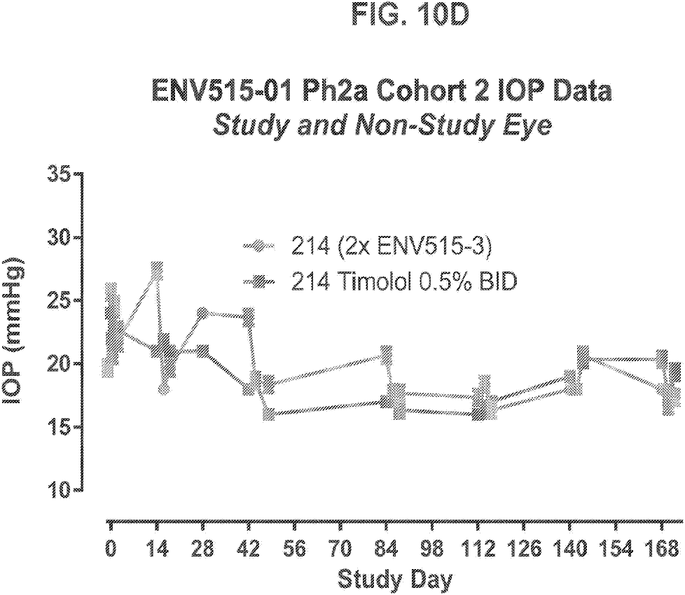

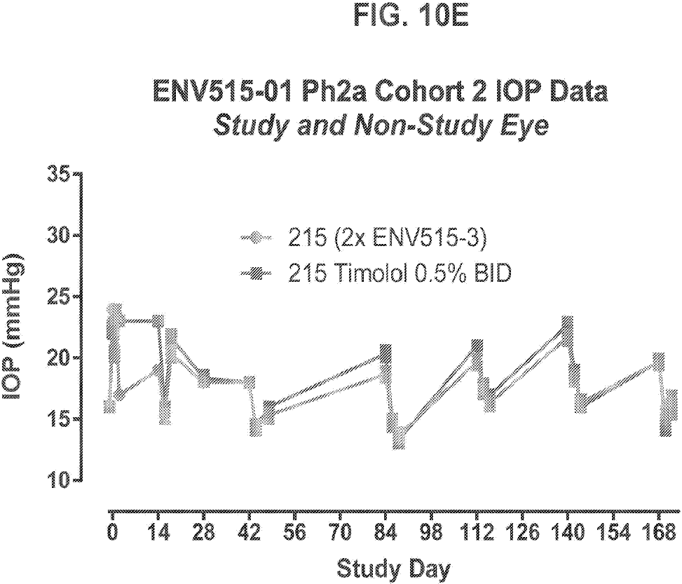

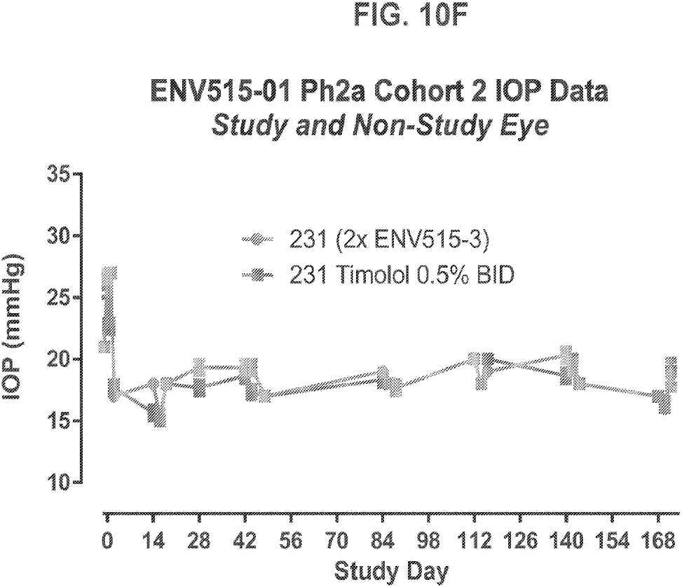

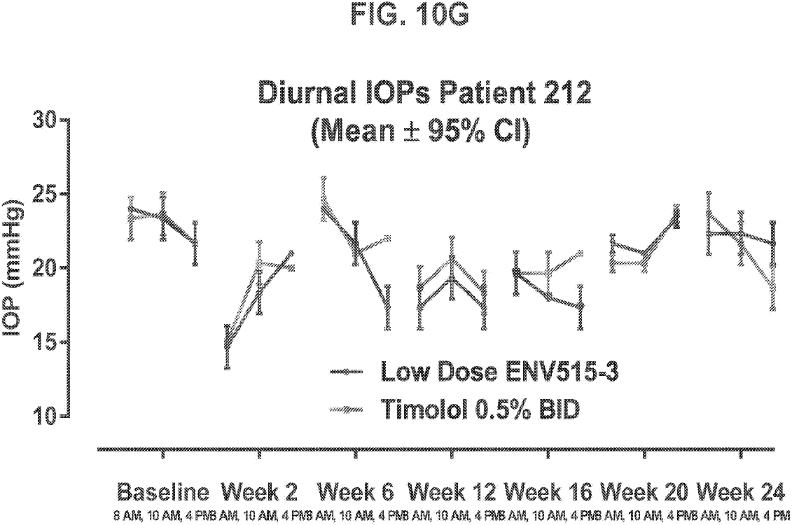

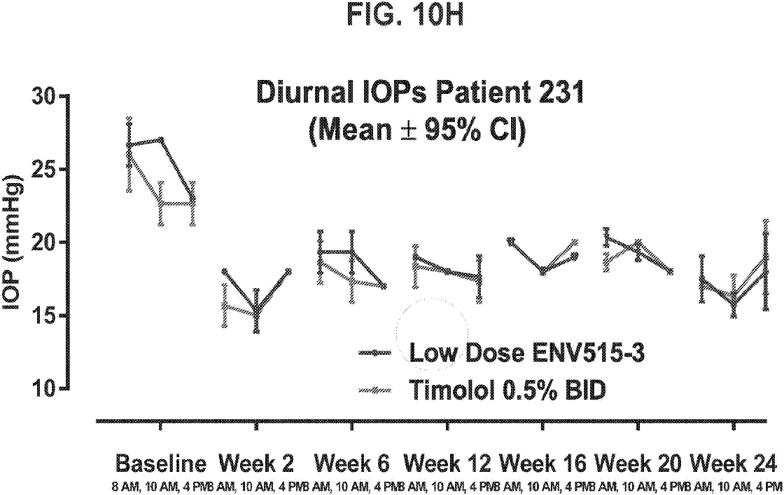

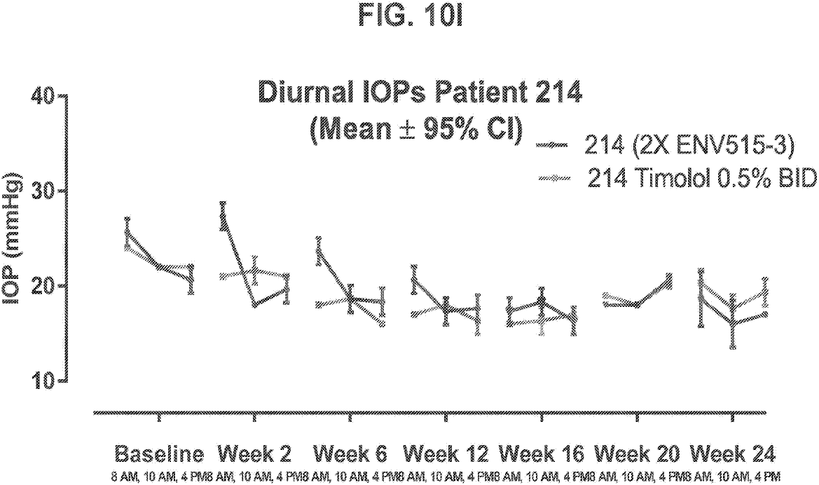

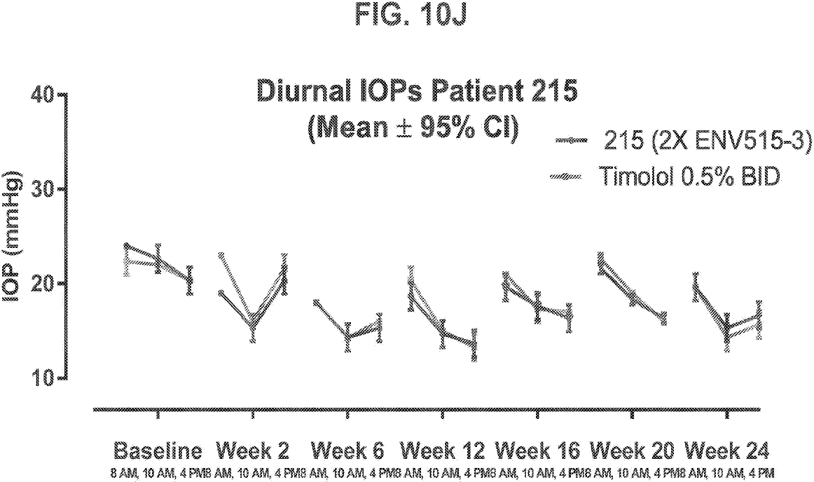

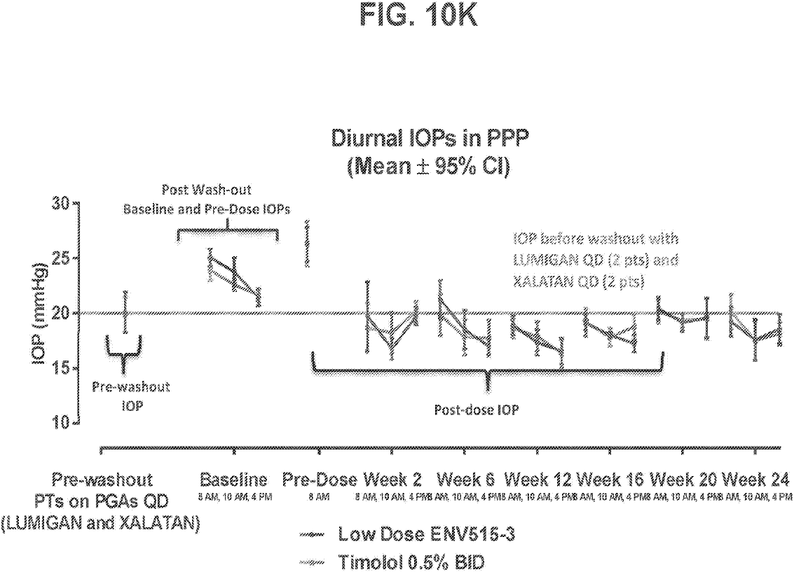

In the Cohort 2 intent-to-treat population (ITT), mean decreases from baseline diurnal IOP over 6 months of the study were observed in all 5 patients with bilateral open-angle glaucoma or ocular hypertension (the study eye, p-values < 0.05 for all dose groups) For the non-study eyes dosed with timolol maleate 0.5% ophthalmic solution, the lOP-lowering treatment effect was comparable to the studied low dose of ENV515-3.
ENV515-3 low dose change from baseline in 8 AM IOP averaged over 6 months in the ITT patient population (all timepoints were weighted equally)
Low dose (28.2 µg travoprost, 2 implants/eye): -6.8 + 3.7 mmHg or -26 % (mean ± SD, n=5 for 6 months, p < 0.001 vs. baseline)
Timolol maleate 0.5% ophthalmic solution change from baseline in 8 AM IOP averaged over 3 months in the ITT patient population (all timepoints were weighted equally).
Timolol maleate 0.5% ophthalmic solution BID: -7.1 ± 4.0 mmHg or -27% (mean ± SD, n=5 for 3 months, p < 0.001 vs. baseline)
These results are further displayed in FIGS. 10A-K. At the time of submission, the ENV515-01 Phase 2a clinical trial is ongoing and patients continue to display IOP lowering effect beyond 6 months. These results indicated robust, sustained and clinically significant IOP lowering effect after a single dose of ENV515-3 (2 implants/eye) that was comparable to active comparator agent timolol maleate 0.5% dosed twice a day into a non-study, sister eye of each patient that extend to 6 months and beyond.

### Example 26. Clinically Significant IOP Lowering Sustained for at Least About 7 Months Following Implant Administration

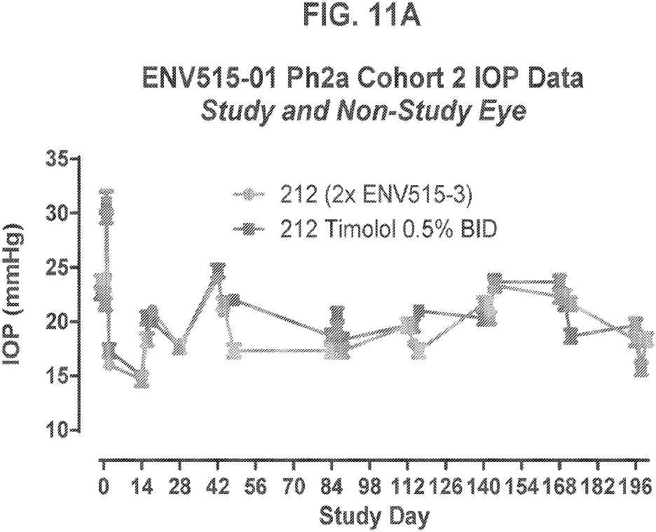

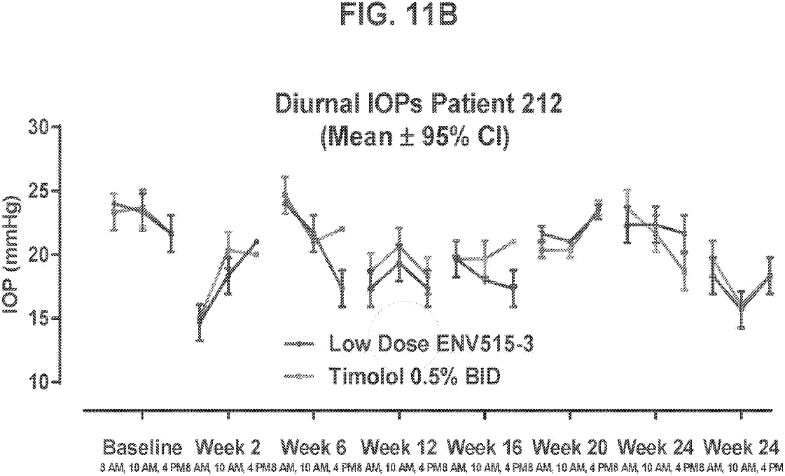

At the time of submission, the ENV515-01 Phase 2a clinical trial is ongoing and patients continue to display IOP lowering effect beyond 6 months One patient evaluated at Month 7 at the time of submission demonstrated robust IOP control without any loss of efficacy at Month 7 of the study (FIG. 11). These results indicated robust, sustained and clinically significant IOP lowering effect after a single dose of ENV515-3 (2 implants/eye) that was comparable to active comparator agent timolol maleate 0.5% dosed twice a day into a non-study, sister eye of each patient that extend to 7 months and beyond.

### Example 27. Hyperemia

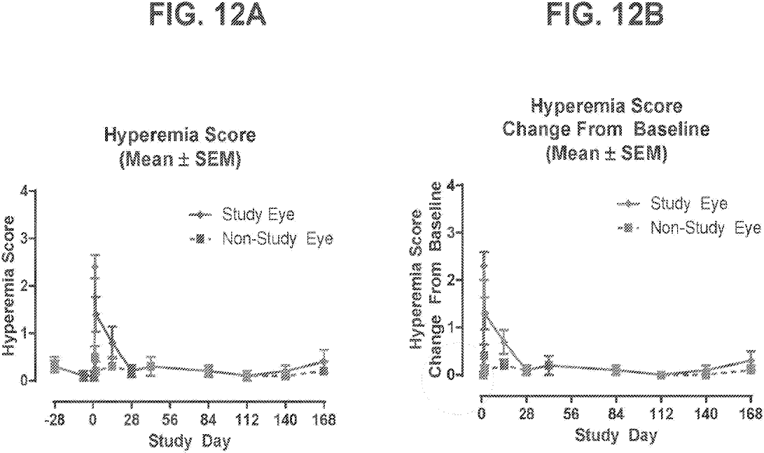

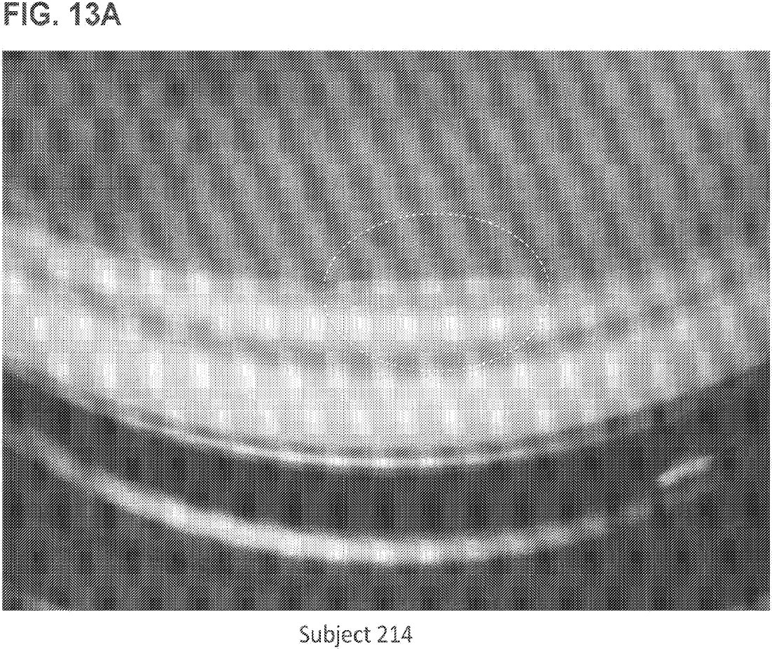

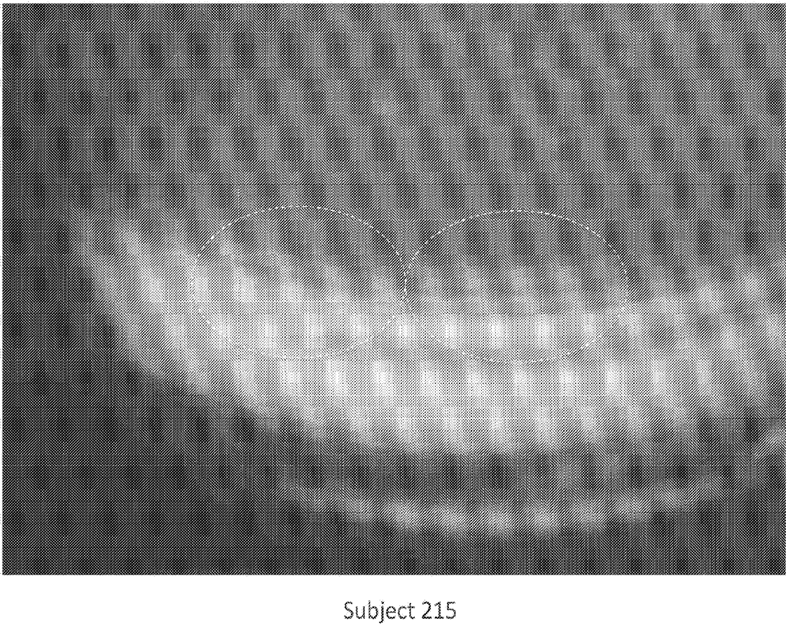

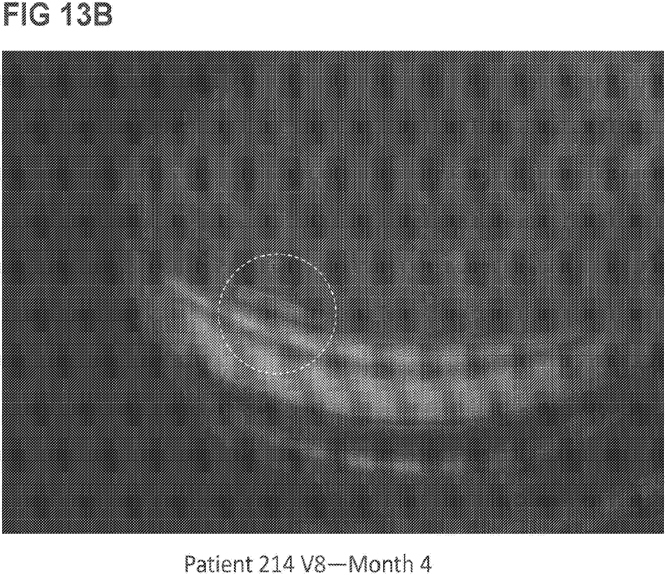

In the ENV515-01 Phase 2a Cohort 2 clinical trial, the extent of hyperemia was evaluated using a high resolution hyperemia scale. An expected increase in early onset hyperemia related to the ENV5IS-3 dosing procedure (intracameral injection) was observed. Surprisingly, beyond 28 days, no hyperemia was not observed above baseline, that was established prior to dosing of the ENV515-3, at generally low levels comparable to the topical timolol maleate 0.5% ophthalmic solution dosed twice a day (See FIGS. 12A and 12B). This is in contrast to the existing literature that demonstrates increased hyperemia for topical ophthalmic prostaglandin analogs such as TRAVATAN (travoprost), LUMIGAN (bimatoprost) and XALATAN (latanoprost). Based on this findings, the authors surprisingly discovered that travoprost and other prostaglandin analogs, when dosed intraocularly, do not cause ocular hyperemia to the same extent as prostaglandin analogs dosed topically (e.g. TRAVATAN, LUMIGAN, XALATAN and others).

### Example 28. Implant Orientation in Iridocorneal Angle

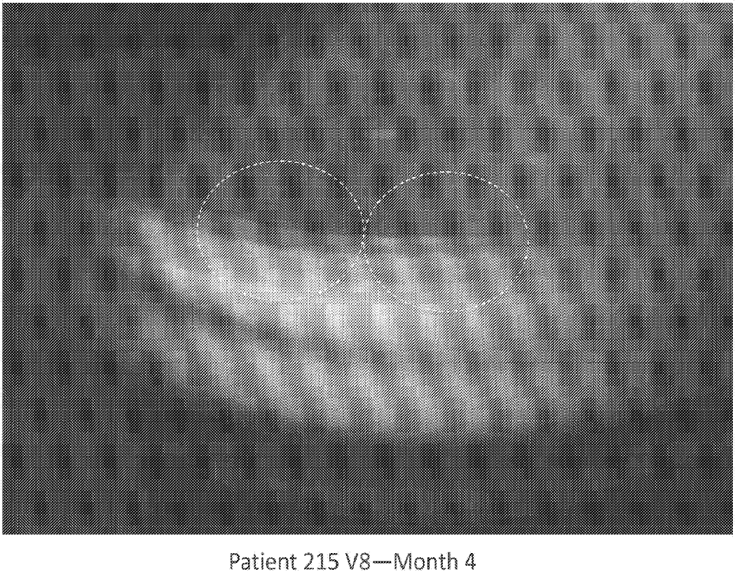

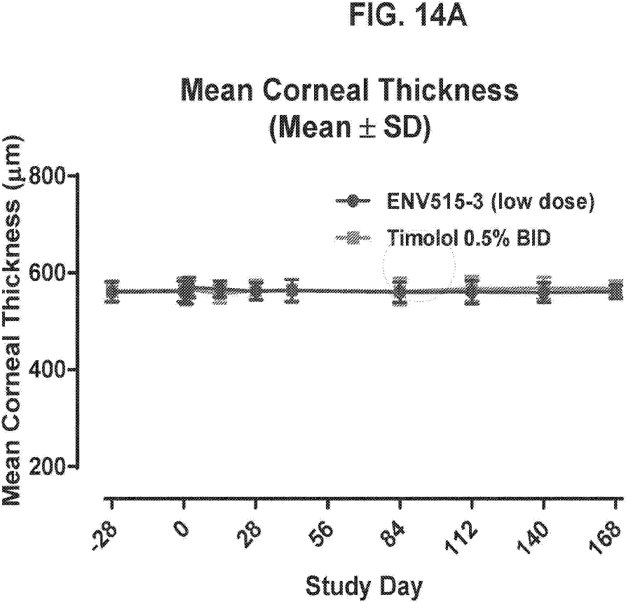

In a ENV515-01 Phase 2a Cohort 2 clinical trial, the location of the ENV515-3 implants was monitored via gonioscopy (FIGS. 13A and 13B) that was conducted as described in the study protocol. The implants localized into the irodocorneal angle to between 6-8 o’clock at the angle in a back-to-back or stacked orientations. No signs of local inflammation or synechia were observed and the implants were very well tolerated.

### Example 29. Safety of ENV515-3 Implants

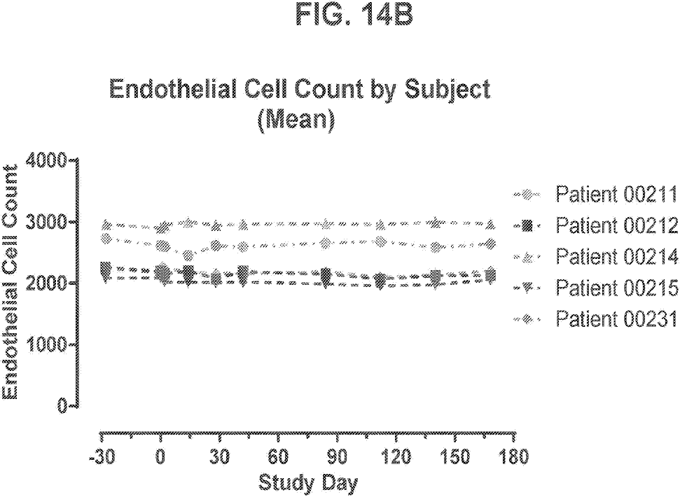

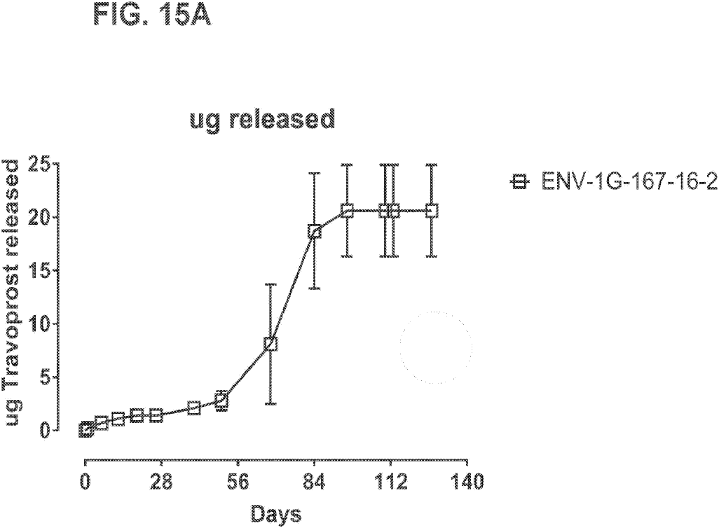

The safety of 2 ENV515-3 implants/eye was evaluated in ENV515-01 Phase 2a Cohort 2 clinical trial conducted in glaucoma patients. There were no serious adverse events (SAEs) in the first 7 months of the study. The ocular adverse events observed in the study were generally mild in nature, occurring mostly early in the study (Table 17 and Table 18). The majority of the adverse events were related to the dosing procedure which involved intracameral injection of ENV515-3. No impact on corneal endothelium was observed in the low dose arm of ENV515-3 (2 implants/eye, FIG. 14). Based on these results, ENV515-3 2 implants/eye were well tolerated and demonstrated a good safety profile.

TABLE 17

Overall Summary of Adverse Events for ENV515-3 Excluding Hyperemia

| Reported Events (summarized by preferred term) | Number of Subjects n(%) | Severity by AE incidence reported | Causality | Latest Day Resolved Post Dose by Incidence |
|---|---|---|---|---|
| Early Onset | | | | |
| Foreign body sensation in eyes | 4 (80) | 3 -Mild 1- Moderate | Injection Dosing Procedure | Day 5 |
| Anterior chamber Flare | 1 (20) | Mild | Injection Dosing Procedure | Day 10 |
| Photophobia | 1 (20) | Mild | Injection Dosing Procedure | Day 5 |
| Iritis | 1 (20) | Mild | Injection Dosing Procedure | Day 3 |
| Punctate keratitis | 1 (20) | Moderate | Injection Dosing Procedure | Day 2 |
| Eye Pain | 1 (20) | Moderate | Injection Dosing Procedure | Day 5 |
| Eyelid oedema | 1 (20) | Mild | Injection Dosing Procedure | Day 14 |
| Ocular discomfort | 1 (20) | Moderate | Injection Dosing Procedure | Day 28 |
| Late Onset | | | | |
| Vision Blurred | 1 (20) | Mild | Unrelated | Resolved |
| Keratitis (Inferior Superficial) | 1 (20) | Mild (reported in both eyes) | TBD at next visit (started 202 days post dose) | Ongoing |

TABLE 18

Classification of Hyperemia Adverse Events for ENV5-3 Dose Arm

| Reported Adverse Events (summarized by coded preferred term) | Number of Incidences Reported | Study Eye or Both Eyes | Severity | Causality | Resolution | Starting Day | Duration of Event in Days |
|---|---|---|---|---|---|---|---|
| Ocular Hyperemia | 2 | Study Eye | Moderate | Injection dosing procedure | Resolved | Same day as dosing | 15 |
| Ocular Hyperemia | 2 | Study Eye | Severe | Injection dosing procedure | Resolved | Same day as dosing | 2 |
| Ocular Hyperemia | 1 | Study Eye | Mild | Unrelated (note: patient also reported an AE of “Worsening of Seasonal Allergies” starting the same day) | Resolved | 15 days post dose | 13 |
| Ocular Hyperemia | 1 | Both Eyes | Mild | Unrelated | Resolved | 40 days post dose | 44 |
| Conjunctival Hyperemia | 1 | Study Eye | Moderate | Injection dosing procedure | Resolved | Same day as dosing | 28 |
| Ocular Hyperemia | 1 | Study Eye | Mild | To be determined | Ongoing | 202 days post dose | TBD |
| Ocular Hyperemia | 1 | Study Eye | Mild | Unrelated | Ongoing | 169 days post dose | TBD |

### Example 28. ENV515-4/5 and ENV515-16-2 Formulations Preparation of Polymer Matrix/Therapeutic Agent Blends

The polymer matrix/therapeutic agent blend was prepared prior to fabrication of implants. Acetone was used to dissolve the polymers and therapeutic agent to create a homogeneous mixture. The polymer blend contained travoprost as the therapeutic agent The resulting solution was aseptically filtered. After filtering, the acetone was evaporated leaving a thin film of homogeneous material. Table 19 details the composition of the various blends.

TABLE 19

Polymer Matrix/Therapeutic Agent Blend Ratios

| Formulation | ID | Size µm (avg.) | Polymer Matrix Blend | R208S (PLA) wt % | R203S (PLA) wt % | RG750S (PLGA) wt % | RG502S (PLGA) wt % | Travoprost wt % |
|---|---|---|---|---|---|---|---|---|
| ENV5I5-16-2 | ENV-LG-167-16-2 | 170 × 210 × 1325 | R208S/R203S/RG 502S 23/67/10 | 15.4 | 44.8 | N/A | 6.7 | 33.1 |
| ENV515-4/5 | ENV-1G-184-12-IB | 200 × 190 × 1500 | R208S/RG750S 85/15 | 50.9 | N/A | 9.0 | N/A | 40.1 |
| | 16075 | 200 × 190 × 1500 | R208S/RG750S 85/15 | 46.8 | N/A | 8.3 | N/A | 45.0 |
| | 16096 | 200 × 190 × 1500 | R208S/RG750S 85/15 | 46.8 | N/A | 8.3 | N/A | 45.0 |
| | 16138 | 200 × 190 × 1500 | R208S/RG750S 85/15 | 46.8 | N/A | 8.3 | N/A | 45.0 |

TABLE 20

ENV515-16-2 and ENV515-4/-5 Content

| Implant | ID | Average µg | STEDV µg | RSD % | Min µg | Max µg | Range µg |
|---|---|---|---|---|---|---|---|
| ENV515-16-2 | ENV-IG-167-16-2 | 14.7 | 0.1 | 0.8 | 14.5 | 14.8 | 0.3 |
| ENV-515-4/5 | ENV-IG-184-12-1B | 23.6 | 0.6 | 2.6 | 22.6 | 24.2 | 1.6 |
| ENV-515-4/5 | 16075 | 28.1 | 1.7 | 5.9 | 24.7 | 31.7 | 7.0 |
| ENV-515-4/5 | 16096 | 28.2 | 1.1 | 3.9 | 26.2 | 29.9 | 3.7 |
| ENV-515-4/5 | 16138 | 30.2 | 1.2 | 3.9 | 27.2 | 32.1 | 4.9 |

### Example 29: Fabrication of Molds

A mold of appropriate dimensions was created with the PRINT™ process The mold had dimensions of 175 µm × 215 µm × 1,390 µm (ENV515-16-2) or 210 µm 200 µm × 1,500 µm (ENV515-4/5)

### Example 30: ENV515-4 and ENV515-5 Implant Fabrication Via PRINT™

ENV515-4 and ENV515-5 are variants of the same formulation, with slightly different manufacturing process leading to the same implant formulation (Table 21 below) Implants were fabricated utilizing the polymer matrix/therapeutic agent blends of Example 28 and the molds of Example 29 Under clean or aseptic conditions, a portion of polymer matrix/therapeutic agent blend was spread over a PET sheet and was heated for approximately 60 to 90 seconds until fluid. Once heated, the blend was covered with the mold of Example 2 which had the desired dimensions. Light pressure was applied using a roller to spread the blend over the mold area. The mold/blend laminate was then passed through a commercially available thermal laminator using the parameters in Table 21 below The blend flowed into the mold cavities and assumed the shape of the mold cavities. The blend was allowed to cool to room temperature and created individual implants in the mold cavities The mold was then removed leaving a two-dimensional array of implants resting on the film. Individual implants were removed from the PET film utilizing forceps.

TABLE 21

Implant Fabrication Conditions: ENV515-4 and EINV515-5

| Parameter | Target (Range) | |
|---|---|---|
| | ENV515-4 | ENV515-5 |
| Batch Size | 5 sheets | 5 sheets |
| Blend Strength (% w/w Travoprost) | 45.0 | 45.0 |
| R208 / RG750S Ratio | 85% R208/15% RG750S | 85% R208/15% RG750S |
| Blend Dispense Volume (mL/sheet) | 6.0 | 5.0 |
| Hot Plate Temperature (°C) | 123 (120 to 125) | 123 (120 to 125) |
| Hot Plate Time (s/sheet) | 60 to 90 | 60 to 90 |
| Laminator Roll Speed (fpm) | 0.10 (± 0.02) | 0.10 (± 0.02) |
| Laminator Pressure (psi) | 80 (± 5) | 80 (± 5) |
| Laminator Temperature (°F) | 385 (384 to 387) | 385 (384 to 387) |
| Laminator Passes | 4 | 3 |

### Example 30. Env515-16-2 and Env515-4/5 Implant Travoprost Drug Release In Vitro

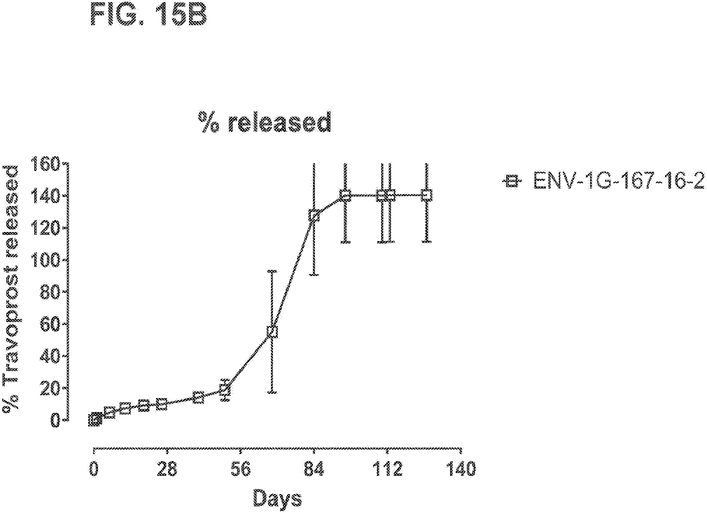

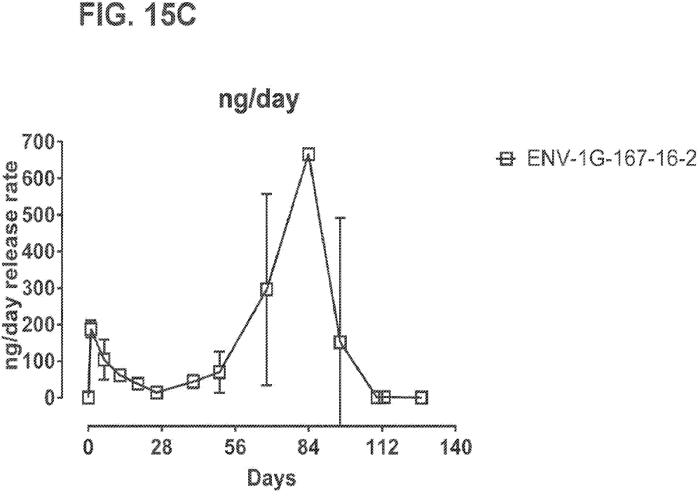

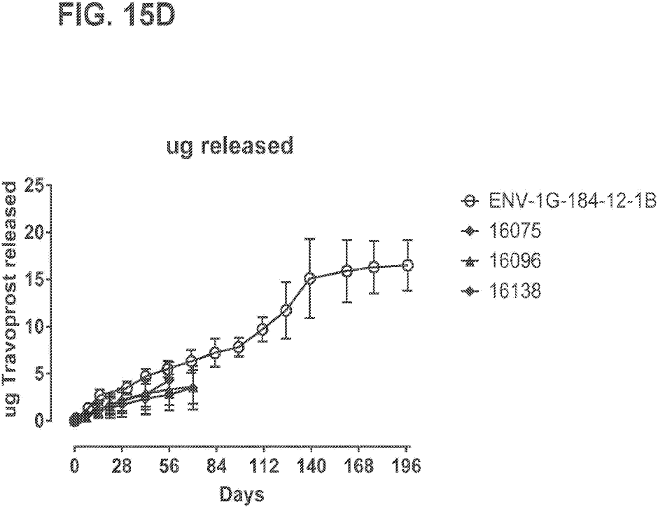

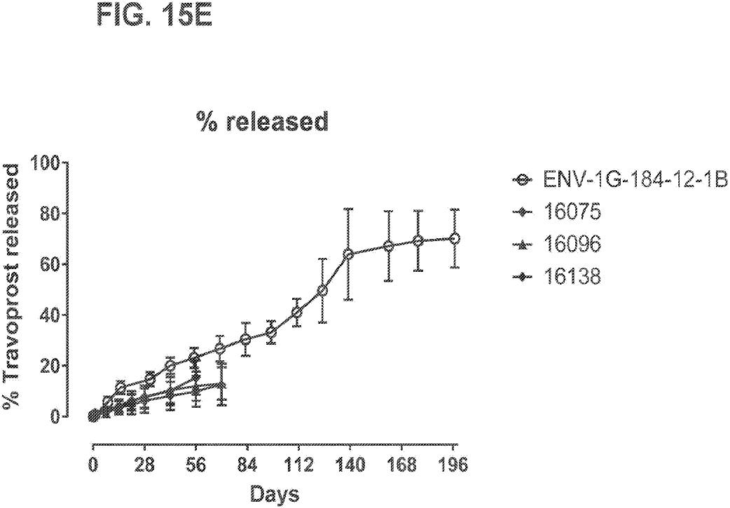

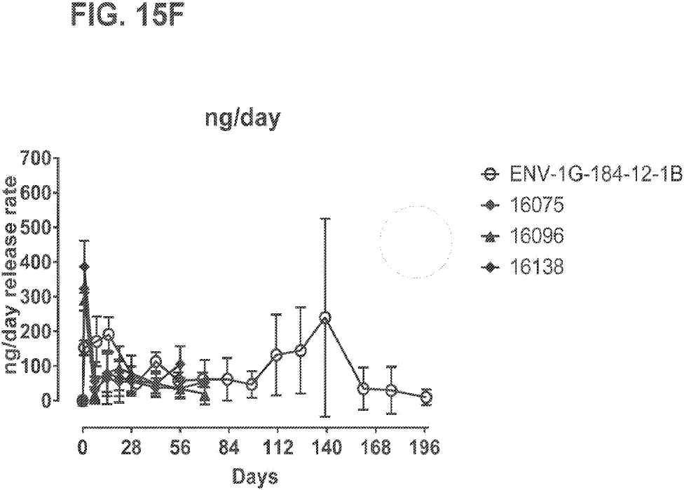

ENV515-16-2 and ENV515-4/5 were evaluated for the release of the travoprost drug in vitro based on method established previously (reference ENV515 first patent application). The data was analyzed and cumulative % of drug released as well as ng of travoprost released per day (FIGS. 15A-F). These profiles indicate more linear release of travoprost drug from the formulation containing PLGA and PLA polymeric excipients. These in vitro data indicate that travoprost release from the ENV515-4/5 formulation extends over a period of ~140 days in this in vitro assay.

### Example 31. Duration Of Env515-4/5 Formulation in Glaucoma Patients Based in Previously Established In Vitro to in Patient Correlation

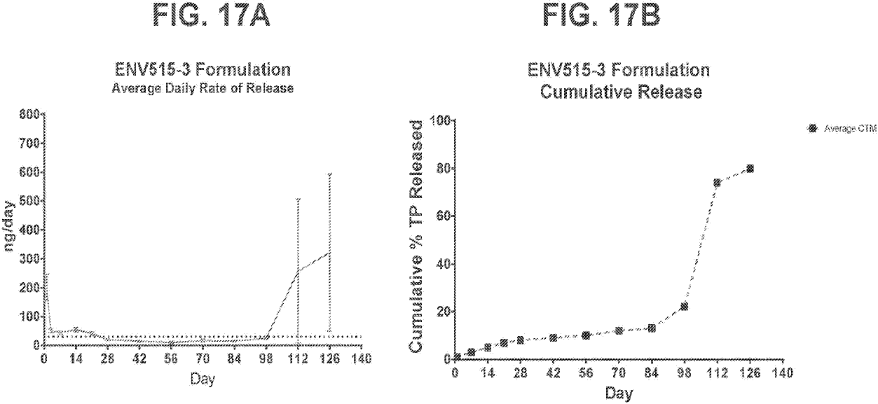

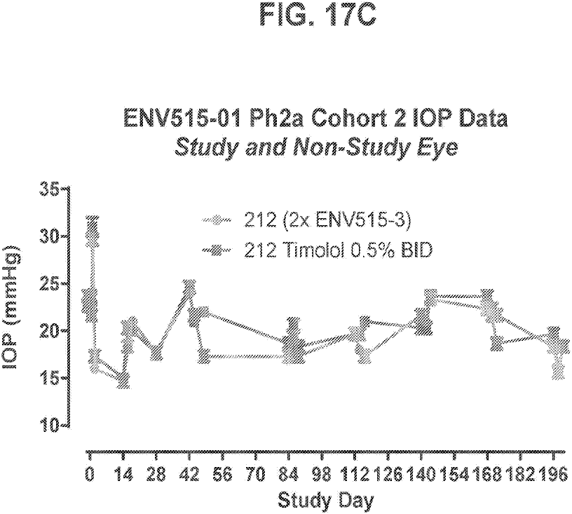

Previously, in vitro travoprost release assay used in Example 30 was also used to characterize the duration of travoprost release for ENV515-3. These in vitro data indicate that travoprost release from the ENV515-3 formulation extends over a period of ~126 days in this in vitro assay. Additionally, the duration of lOP-lowering treatment effect for ENV515-3 was established in glaucoma patients to be at least 7 months or 196 days (Examples 21 to 27 and FIGS. 17A-D). Based on the duration of travoprost release from the ENV515-3 formulation in vitro and its established duration of IOP lowering effect in glaucoma patients, in vitro to in vivo duration correlation coefficient was established ENV515-3 IOP lowering duration in glaucoma patients/ENV515-3 travoprost release in vitro = 196 days in glaucoma patients/126 days in vitro = ~1.6 factor. For the ENV515-4/5 formulation, its duration of travoprost release in vitro occurred over 140 days in vitro. Based on the in vitro to in vivo duration correlation coefficient of 1.6x, the duration of IOP lowering effect of the ENV515-4/5 formulation in glaucoma patients is ~224 days or 8 months

### Example 32. IOP-Lowering Efficacy of Env515-4/5 in Beagle Dog

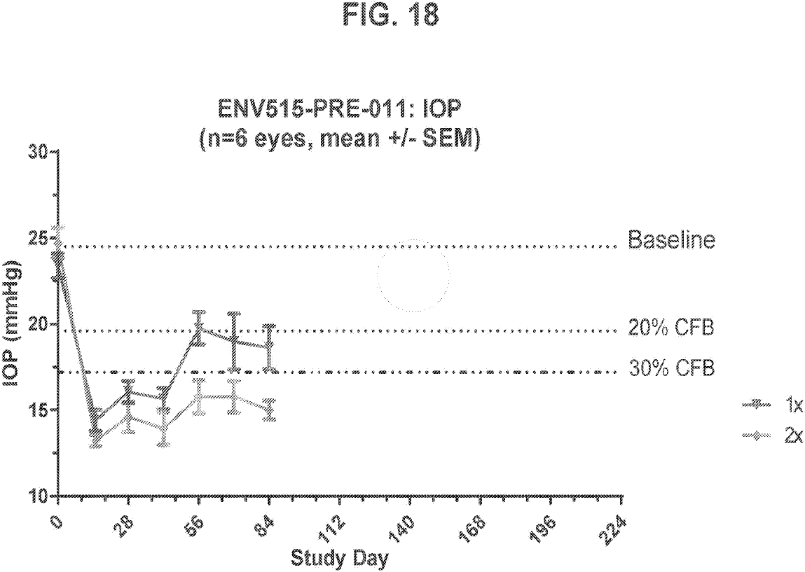

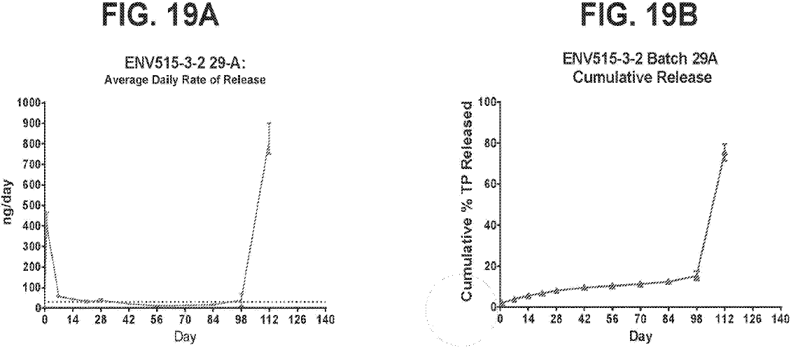

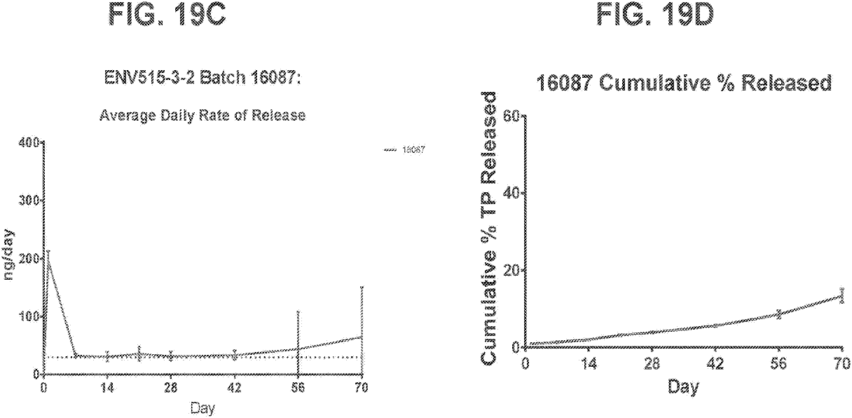

ENV515-4/5 formulation was tested for its lOP-lowering efficacy in spontaneously hypertensive Beagle dog (FIG. 18) with 1 and 2 implants dosed per eye. In this 3-month study, ENV515-4 demonstrated robust, sustained, clinically significant IOP lowering treatment effect

### Example 33. Non-Swelling Nature of ENV515-4/5 and ENV515-16-2 Formulations

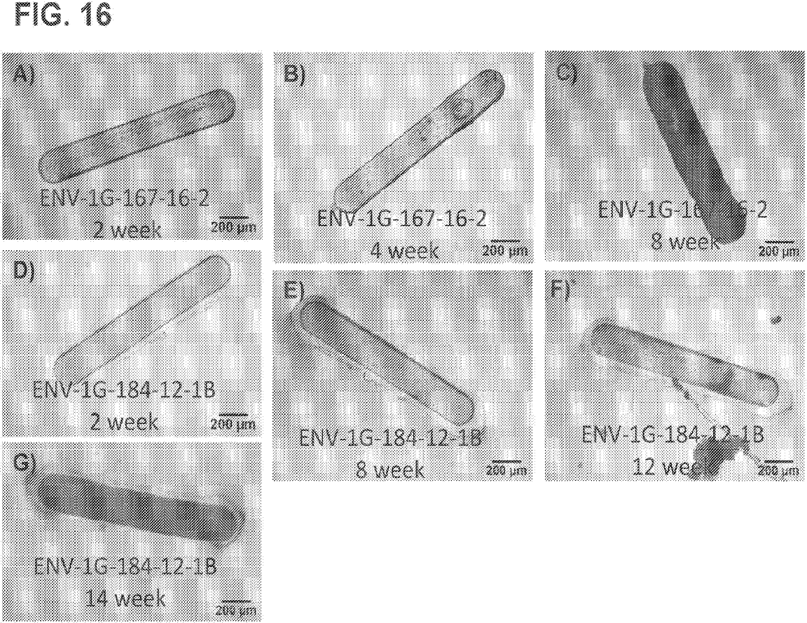

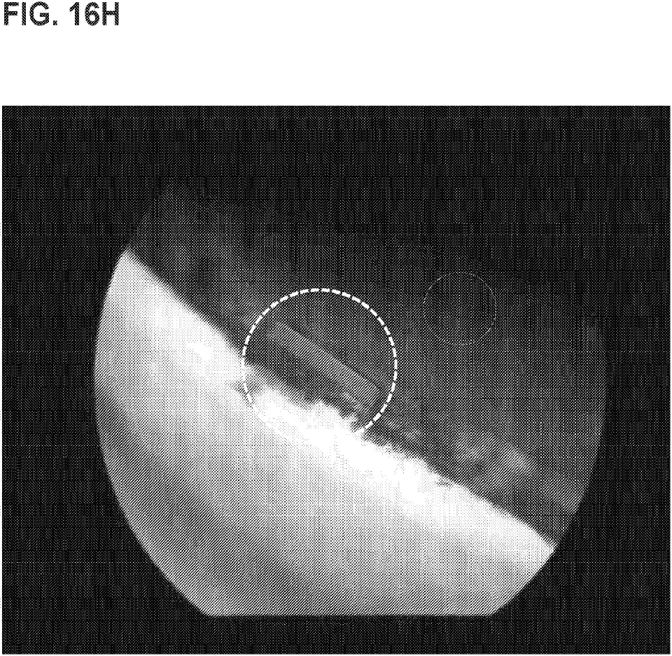

The swelling nature of the ENV515-16-2 and ENV515-4/5 formulations was evaluated by optical imaging during in vitro travoprost release assay and also in vivo in dog IOP study (Examples 30 and 32). PLGA containing formulations ordinarily swell and increase in volume when exposed to aqueous environment as in the in vitro assay and in vivo. Surprisingly, it was discovered that ENV515-16-2 and ENV515-4/5 did not swell in vitro (FIGS. 16A-G). Additionally, when gonioscopy exams were carried out in Beagle dogs during the dog IOP study during which the ENV515-4/5 implants were observed directly in the dog anterior chamber, no swelling was observed in vivo either (FIG. 16H).

TABLE 22

Non-Swelling Data

| ID | 2 weeks | 4 weeks | 6 weeks | 8 weeks | 12 weeks | 14 weeks |
|---|---|---|---|---|---|---|
| ENV-1G-167-16-2 | 205 | 205 | - | 256 | - | - |
| ENV-1G-184-12-1B | 203 | - | 200 | 202 | 203 | 227 |

### Example 34. ENV515-3-2 Formulations: Preparation of Polymer Matrix/Therapeutic Agent Blends

The polymer matrix/therapeutic agent blend was prepared prior to fabrication of implants. Acetone was used to dissolve the polymers and therapeutic agent to create a homogeneous mixture. The polymer blend contained travoprost as the therapeutic agent. The resulting solution was aseptically filtered. After filtering, the acetone was evaporated leaving a thin film of homogeneous material. Tables 23 and 24 detail the composition of the various blends.

TABLE 23

Polymer Matrix/Therapeutic Agent Blend Ratios

| Formulation | ID | Size µm (avg.) | Polymer Matrix Blend | R208S (PLA) wt % | R203S (PLA) wt % | Travoprost wt % |
|---|---|---|---|---|---|---|
| 515-3-2 | ENV-1G-184-18-29A | 200 × 190 × 1500 | R208S/R203S 67/33 | 44.2 | 21.8 | 34.0 |
| | 16087 | 200 × 190 × 1500 | R208S/R203S 67/33 | 40.9 | 20.1 | 39.0 |
| | 16100 | 200 × 190 × 1500 | R208S/R203S 67/33 | 40.9 | 20.1 | 39.0 |

TABLE 24

ENV515-3-2 Content

| ID | Average µg | STEDV µg | RSD % | Min µg | Max µg | Range µg |
|---|---|---|---|---|---|---|
| 16087 | 26.5 | 1.6 | 6.0 | 23.5 | 30.3 | 6.7 |
| 16100 | 25.3 | 1.0 | 3 8 | 23.4 | 27.4 | 4.0 |

### Example 35: Fabrication of Molds

A mold of appropriate dimensions was created with the PRINT™ process. The mold had dimensions of 210 µm × 200 µm × 1,500 µm {ENV515-3-2) or 210 µm × 200 µm × 1,500 µm (ENV515-4/5).

### Example 36: ENV515-3-2 Implant Fabrication Via PRINT™

Implants were fabricated utilizing the polymer matrix/therapeutic agent blends of Example 28 and the molds of Example 29. Under clean or aseptic conditions, a portion of polymer matrix/therapeutic agent blend was spread over a PET sheet and was heated for approximately 60 to 90 seconds until fluid. Once heated, the blend was covered with the mold of Example 2 which had the desired dimensions. Light pressure was applied using a roller to spread the blend over the mold area. The mold/blend laminate was then passed through a commercially available thermal laminator using the parameters in Table 25 below. The blend flowed into the mold cavities and assumed the shape of the mold cavities The blend was allowed to cool to room temperature and created individual implants in the mold cavities. The mold was then removed leaving a two-dimensional array of implants resting on the film Individual implants were removed from the PET film utilizing forceps.

TABLE 25

Implant Fabrication Conditions: ENV515-3-2

| Parameter | Target (Range) | | |
|---|---|---|---|
| | ENV515-3-2 | ENV515-4 | ENV515-5 |
| Batch Size | 5 sheets | 5 sheets | 5 sheets |
| Blend Strength (%w/w Travoprost) | 39.0 | 45.0 | 45.0 |
| Polymer Ratio | 67% R208/33%R203S | 85% R208/15%RG750S | 85% R208/15% RG750S |
| Blend Dispense Volume (mL/sheet) | 6.0 | 6.0 | 5.0 |
| Hot Plate Temperature (°C) | 123(120 to 125) | 123(120 to 125) | 123(120 to 125) |
| Hot Plate Time (s/sheet) | 60 to 90 | 60 to 90 | 60 to 90 |
| Laminator Roll Speed (fpm) | 0.20 (= 0.02) | 0.10 (±0.02) | 0.10 (± 0.02) |
| Laminator Pressure (psi) | 80 (± 5) | 80 (± 5) | 80 (± 5) |
| Laminator Temperature (°F) | 376 (375 to 378) | 385 (384 to 387) | 385 (384 to 387) |
| Laminator Passes | 4 | 4 | 3 |

### Example 37. ENV515-3-2 Implant Travoprost Drug Release in Vitro

ENV515-3-2 formulation was evaluated for the release of the travoprost drug in vitro based on method established previously (reference ENV515 first patent application). The data was analyzed and cumulative % of drug released as well as ng of travoprost released per day (FIGS. 19A-D) These in vitro data indicate that travoprost release from the ENV515-3-2 formulation extends over a period of ~112 days in this in vitro assay

### Example 38. Duration of ENV515-4/5 Formulation in Glaucoma Patients Based in Previously Established In Vitro to in Patient Correlation

Previously, in vitro travoprost release assay used in Example 30 was also used to characterize the duration of travoprost release for ENV515-3. These in vitro data indicate that travoprost release from the ENV515-3 formulation extends over a period of ~126 days in this in vitro assay. Additionally, the duration of IOP-lowering treatment effect for ENV515-3 was established in glaucoma patients to be at least 7 months or 196 days (Examples 21 to 27 and FIG. 17) Based on the duration of travoprost release from the ENV515-3 formulation in vitro and its established duration of IOP lowering effect in glaucoma patients, in vitro to in vivo duration correlation coefficient was established: ENV515-3 IOP lowering duration in glaucoma patients/ENV515-3 travoprost release in vitro - 196 days in glaucoma patients/126 days in vitro = ~1.6 factor. For the ENV515-3-2 formulation, its duration of travoprost release in vitro occurred over 112 days in vitro. Based on the in vitro to in vivo duration correlation coefficient of 1.6x, the duration of IOP lowering effect of the ENV515-3-2 formulation in glaucoma patients is ~179 days or > 6 months.

### Example 39. IOP-Lowering Efficacy of Env515-3-2 in Beagle Dog

EVN5152 formulation batch 29A was tested for its IOP-lowering efficacy in spontaneously hypertensive Beagle dog (FIG. 20) with 2 implants dosed per eye. In this study, ENV515-3-2 demonstrated robust, sustained, clinically significant IOP lowering treatment effect that lasted greater than 205 days or greater than 7 months.

### Example 40. IOP-Lowering Efficacy of Env515-3-1

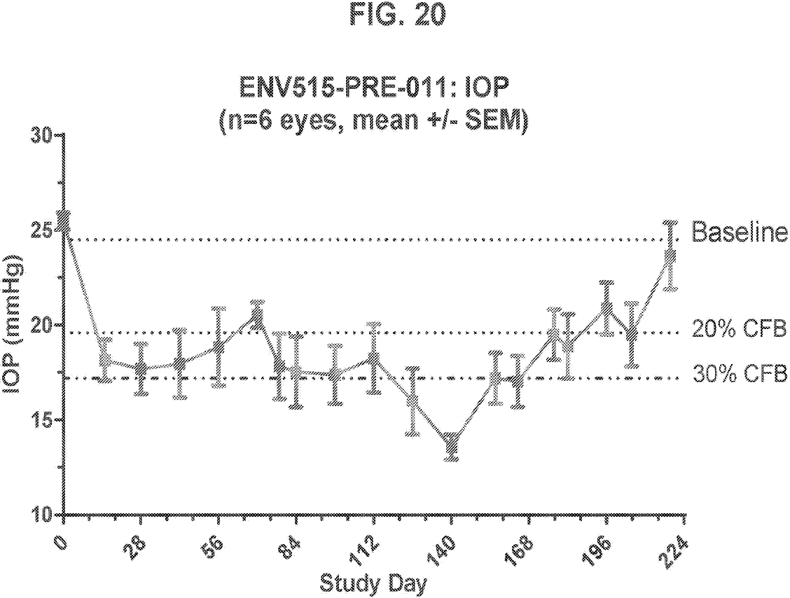

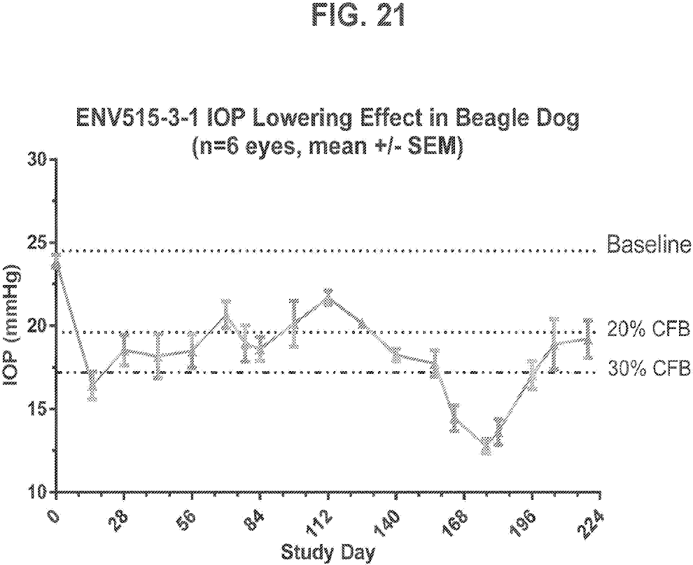

ENV515-3-1 formulation, a close variant of ENV515-3 and ENV515-3-2 differing only in size was prepared and tested for its TOP-lowering efficacy in spontaneously hypertensive Beagle dog (FIG. 21) with 3 implants dosed per eye. In this study, ENV515-3-1 demonstrated robust, sustained, clinically significant IOP lowering treatment effect that lasted greater than 224 days or greater than 8 months.

### Prophetic Example 1: ENV515-3-2 Study in Glaucoma Patients

Clinical efficacy and safety is evaluated in randomized, active comparator controlled study in which patients with glaucoma are treated with active comparator timolol 0.5% ophthalmic solution BID in control arm 1, TRAVATAN Z ophthalmic solution QD in control arm 2, and two dose levels of ENV515-3-2, 1 and 2 implants per eye in the study eye dosed unilaterally in investigational product arm 3 and 4 (i.e. this is a parallel, four-arm study).

Prior to dosing with active comparator and ENV515-3-2, patients are washed out of all of their lOP-lowering medications, i.e. all of patients IOP-lowering medications are withdrawn and not used. Following 6-week washout period, patients’ IOP is at baseline level of 25-28 mmHg at 8 AM, with corresponding diurnal IOP lowering at 10 AM and 4 PM. Following washout, timolol 0.5% BID is administered as indicated twice every day in control arm 1; TRAVATAN Z is administered as indicated in controlled arm 2; ENV515-3-2 is administered once on Day 1 with one implant per eye in the investigational product arm 3; and ENV515-3-2 is administered once on Day 1 with two implants/eye in the investigational arm 4. The mean change in 8 AM IOP from post-washout, pre-dose baseline and mean change in mean diurnal IOP from post-washout, pre-dose (mean of 8 am, 10 am and 4 pm IOP measurements) baseline is assessed over 12 months. All adverse events are tracked and evaluated, including adverse event of hyperemia, iris and pigmented tissue discoloration. The diurnal IOP is evaluated at 2 weeks, 6 weeks and 12 weeks after Day 1 dosing with ENV515-3-2 and on Month 4, 5, 6, 7, 8, 9, 10, 11 and 12. It is observed that ENV515-3-2 maintains statistically significant and clinically meaningful decrease in 8 AM IOP baseline with magnitude of 20 to 30% change from baseline for a period of approximately greater than 6 months and for some patients for a period of 7 to 10 months or longer in both treatment arms 3 and 4. It is observed that the incidence of adverse events of hyperemia score after 28 days and the incidence of increased pigmentation of iris and eyelids is decreased in ENV515-3-2 treatment arms compared to TRAVATAN-Z control arm.

### Prophetic Example 2: ENV515-4 Study in Glaucoma Patients

Clinical efficacy and safety is evaluated in randomized, active comparator controlled study in which patients with glaucoma are treated with active comparator timolol 0.5% ophthalmic solution BID in control arm 1, TRAVATAN Z ophthalmic solution QD in control arm 2, and two dose levels of ENV515-4, 1 and 2 implants per eye in the study eye dosed unilaterally in investigational product arm 3 and 4 (i.e. this is a parallel, four-arm study).

Prior to dosing with active comparator and ENV515-4, patients are washed out of all of their IOP-lowering medications; i.e. all of patients IOP-lowering medications are withdrawn and not used. Following 6-week washout period, patients’ IOP is at baseline level of 25-28 mmHg at 8 AM, with corresponding diurnal IOP lowering at 10 AM and 4 PM. Following washout, timolol 0.5% BID is administered as indicated twice every day in control arm 1; TRAVATAN Z is administered as indicated in controlled arm 2; ENV515-4 is administered once on Day 1 with one implant per eye in the investigational product arm 3; and ENV515-4 is administered once on Day 1 with two implants/eye in the investigational arm 4. The mean change in 8 AM IOP from post-washout, pre-dose baseline and mean change in mean diurnal IOP from post-washout, pre-dose (mean of 8 am, 10 am and 4 pm IOP measurements) baseline is assessed over 12 months. All adverse events arc tracked and evaluated, including adverse event of hyperemia, iris and pigmented tissue discoloration. The diurnal IOP is evaluated at 2 weeks, 6 weeks and 12 weeks after Day 1 dosing with ENV515-3-2 and on Month 4, 5, 6, 7, 8, 9, 10, 11 and 12. It is observed that ENV515-3-2 maintains statistically significant and clinically meaningful decrease in 8 AM IOP post-washout, pre-dose baseline with magnitude of 20 to 30% change from baseline as well as statistically significant and clinically meaningful change from post-washout, pre-dose baseline in mean diurnal IOP for a period of approximately greater than 6 months and for some patients for a period of 7 to 10 months or longer in both treatment arms 3 and 4. It is observed that the incidence of adverse events of hyperemia score after 28 days and the incidence of increased pigmentation of iris and eyelids is decreased in ENV515-4 treatment arms compared to TRAVATAN-Z control arm

### Prophetic Example 3: ENV515-5 Study in Glaucoma Patients

Clinical efficacy and safety is evaluated in randomized, active comparator controlled study in which patients with glaucoma are treated with active comparator timolol 0.5% ophthalmic solution BID in control arm 1, TRAVATAN Z ophthalmic solution QD in control arm 2, and two dose levels of ENV515-5, 1 and 2 implants per eye in the study eye dosed unilaterally in investigational product arm 3 and 4 (i.e. this is a parallel, four-arm study).

Prior to dosing with active comparator and ENV515-5, patients are washed out of all of their IOP-lowering medications, i.e. all of patients lOP-lowering medications are withdrawn and not used. Following 6-week washout period, patients’ IOP is at baseline level of 25-28 mmHg at 8 AM, with corresponding diurnal IOP lowering at 10 AM and 4 PM. Following washout, timolol 0.5% BID is administered as indicated twice every day in control arm 1; TRAVATAN Z is administered as indicated in controlled arm 2; ENV515-5 is administered once on Day 1 with one implant per eye in the investigational product arm 3, and ENV515-5 is administered once on Day 1 with two implants/eye in the investigational arm 4. The mean change in 8 AM IOP from post-washout, pre-dose baseline and mean change in mean diurnal IOP from post-washout, pre-dose (mean of 8 am, 10 am and 4 pm IOP measurements) baseline is assessed over 12 months. All adverse events are tracked and evaluated, including adverse event of hyperemia, iris and pigmented tissue discoloration. The diurnal IOP is evaluated at 2 weeks, 6 weeks and 12 weeks after Day 1 dosing with ENV515-5 and on Month 4, 5, 6, 7, 8, 9, 10, 11 and 12. It is observed that ENV515-5 maintains statistically significant and clinically meaningful decrease in 8 AM IOP post-washout, pre-dose baseline with magnitude of 20 to 30% change from baseline as well as statistically significant and clinically meaningful change from post-washout, pre-dose baseline in mean diurnal IOP for a period of approximately greater than 6 months and for some patients for a period of 7 to 10 months or longer in both treatment arms 3 and 4. It is observed that the incidence of adverse events of hyperemia score after 28 days and the incidence of increased pigmentation of iris and eyelids is decreased in ENV515-4 treatment arms compared to TRAVATAN-Z control arm.

As generally applicable to the Examples 1, 2 and 3 above, travoprost ophthalmic solution such as TRAVATAN Z has been reported to cause changes to pigmented tissues. The most frequently reported changes have been increased pigmentation of the iris, periorbital tissue (eyelid) and eyelashes. Pigmentation is expected to increase as long as travoprost is administered The pigmentation change is due to increased melanin content in the melanocytes rather than to an increase in the number of melanocytes. After discontinuation of travoprost, pigmentation of the iris is likely to be permanent, while pigmentation of the periorbital tissue and eyelash changes have been reported to be reversible in some patients. Patients who receive treatment should be informed of the possibility of increased pigmentation. The long term effects of increased pigmentation are not known. Iris color change may not be noticeable for several months to years. Typically, the brown pigmentation around the pupil spreads concentrically towards the periphery of the iris and the entire iris or parts of the iris become more brownish. Iris pigmentation occurs with an approximate frequency of 1-4%.

As also generally applicable to the Examples 1, 2 and 3 above, the most common adverse reaction observed in controlled clinical studies with TRAVATAN® (travoprost ophthalmic solution) 0.004% and TRAVATAN Z® (travoprost ophthalmic solution) 0.004% was ocular hyperemia which was reported in 30 to 50% of patients.

The ENV515-3-2, ENV515-4 and ENV515-5 with one and two implants per eye demonstrated a lesser rate of these adverse events in when compared to the rates of these events in the TRAVATAN Z control arms in the Examples 1, 2, and 3 above.

IMPLANT FORMULATION SUMMARY TABLE

| ID | Formulation¹ | Polymer (wt%)² | Polymer (µg)³ | API (%wt)⁴ | API (µg)⁵ | Mold Dimensions (µm)⁶, (Width x Height x Length) | Implant Dimensions (µm)⁷, (Width x Height x Length) |
|---|---|---|---|---|---|---|---|
| ENV515-1 | R203S/R208, (ratio 33/67) | 67.0% | 83.6 | | 41.2 | 215 × 230 × 2,925 | 175 × 215 × 2,780 |
| | | (R203S: 22.1 wt%) | | 33.0% | | | |
| | | (R208: 44.9 wt%) | | | | | |
| ENV515-3 | R203S/R208, (ratio 33/67) | 68.4% | 29.4 | | 13.6 | 145 × 190 × 1,500 | 132 × 180 × 1438 |
| | | (R203S: 22.6 wt%) | | 31.6% | | | |
| | | (R208 45.8 wt%) | | | | | |
| ENV515-3-2 | R203S/R208 (ratio 33/67) | 61.6% (R203S: 20.3 wt%). (R208S: 41.3 wt%). | 41.9 | 38.4% | 26.1 | 210 × 200 × 1,550 | 200 × 190 × 1500 |
| ENV515-4/5 | RG750S/R208 (ratio 15/85) | 58.8% (RG750S: 8.8 wt%), (R208S: 50.0 wt%), | 40.0 | 41.2% | 28.0 | 210 × 200 × 1,550 | 200 × 190 × 1500 |
| ENV515-16-2 | RG752S/R203S/ R208 (ratio 10/67/23) | 68.8% (RG502S:6.9 wt%), (R203S: 46.1 wt%), (R208S: 15.8 wt%), | 32.4 | 31.2% | 14.7 | 175 × 215 × 1,390 | 170 × 210 × 1,325 |

1. The ratios of polymers in the polymer matrix can vary by about 20%.
2. The wt% of polymers in the polymer matrix can vary by about 20%.
3. The mass (µm) of polymers in the implant can vary by about 20%.
4. The wt % of API in the implant can vary by about 20%
5. The mass (µm) of API in the implant can vary by about 20%.
6. The mold dimensions used to fabricate the implant can vary by about 20% in any dimension.
7. The dimension of the implant can vary by about 20% in any dimension.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed:

1-177. (canceled)

178. A method for lowering intraocular pressure in a subject in need thereof, the method comprising administering at least one intracameral implant to the anterior chamber of the eye of the subject; wherein the intracameral implant comprises a biodegradable polymer matrix and a therapeutic agent homogenously dispersed therein; wherein the intracameral implant achieves a sustained release of the therapeutic agent into the aqueous humor; and wherein the therapeutic agent is released at a concentration below an EC50 calculated for the therapeutic agent when administered without the intracameral implant.

179. The method of claim 178, wherein the biodegradable polymer matrix comprises as a wt % per implant:
(i) 22 ± 5% of a biodegradable poly(D,L-lactide) homopolymer having an inherent viscosity of 0.25 to 0.35 dL/g; and
(ii) 45 ± 5% of a biodegradable poly(D,L-lactide) homopolymer having an inherent viscosity of 1.8 to 2.2 dL/g;
wherein the inherent viscosity is measured at 0.1% w/v in CHCl₃ at 25° C. with a Ubbelhode size 0c glass capillary viscometer.

180. The method of claim 178, wherein the biodegradable polymer matrix comprises as a wt % per implant:
(i) 9 ± 5% of an ester end-capped biodegradable poly(D,L-lactide-co-glycolide) copolymer having an inherent viscosity of about 0.8 to about 1.2 dL/g; and
(ii) 49 ± 5% of an ester end-capped biodegradable poly(D,L-lactide) homopolymer having an inherent viscosity of about 1.8 to about 2.2 dL/g;
wherein the inherent viscosity is measured at 0.1% w/v in CHCl₃ at 25° C. with a Ubbelhode size 0c glass capillary viscometer.

181. The method of claim 178, wherein the therapeutic agent is a prostaglandin, a prostaglandin analog, a prostamide, a prostamide analog, or a combination of two or more thereof.

182. The method of claim 178, wherein the therapeutic agent is latanoprost, travoprost, bimatoprost, tafluprost, unoprostone isopropyl, or a combination of two or more thereof.

183. The method of claim 178, wherein the therapeutic agent is present in an amount of about 10 ug to about 35 ug per implant.

184. A method for lowering intraocular pressure in an eye of a subject, the method comprising administering travoprost to the anterior chamber of the eye of the subject, thereby achieving a level of travoprost acid in the aqueous humor of the eye which is at least 8× lower than the EC50 value of travoprost acid on its molecular target, and wherein clinically significant lowering of intraocular pressure is sustained.

185. The method of claim 184, wherein the travoprost is administered via an intracameral implant.

186. The method of claim 185, wherein the intracameral implant comprises a biodegradable polymer matrix, where the biodegradable polymer matrix comprises as a wt % per implant:
(i) 7 ± 5% of an ester end-capped biodegradable poly(D,L-lactide-co-glycolide) copolymer having an inherent viscosity of about 0.16 to about 0.24 dL/g;
(ii) 15 ± 5% of an ester end-capped biodegradable poly(D,L-lactide-co-glycolide) homopolymer having an inherent viscosity of about 0.25 to about 0.35 dL/g; and
(iii) 48 ± 5% of an ester end-capped biodegradable poly(D,L-lactide) homopolymer having an inherent viscosity of about 1.8 to about 2.2 dL/g;
wherein the inherent viscosity is measured at 0.1% w/v in CHCl₃ at 25° C. with a Ubbelhode size 0c glass capillary viscometer.

187. The method of claim 185, wherein the intracameral implant comprises a biodegradable polymer matrix, where the biodegradable polymer matrix comprises as a wt % per implant:
(i) 22 ± 5% of a biodegradable poly(D,L-lactide) homopolymer having an inherent viscosity of 0.25 to 0.35 dL/g; and
(ii) 45 ± 5% of a biodegradable poly(D,L-lactide) homopolymer having an inherent viscosity of 1.8 to 2.2 dL/g;
wherein the inherent viscosity is measured at 0.1% w/v in CHCl₃ at 25° C. with a Ubbelhode size 0c glass capillary viscometer.

188. The method of claim 185, wherein the therapeutic agent is present in an amount of about 10 ug to about 35 ug per implant.

189. A pharmaceutical composition comprising a biodegradable polymer matrix and a therapeutic agent homogenously dispersed within the biodegradable polymer matrix; wherein:
(a) the biodegradable polymer matrix comprises:
(i) 9 ± 5% of an ester end-capped biodegradable poly(D,L-lactide-co-glycolide) copolymer having an inherent viscosity of about 0.8 to about 1.2 dL/g, and
(ii) 49 ± 5% of an ester end-capped biodegradable poly(D,L-lactide) homopolymer having an inherent viscosity of about 1.8 to about 2.2 dL/g;
(b) the biodegradable polymer matrix comprises:
(i) 7 ± 5% of an ester end-capped biodegradable poly(D,L-lactide-co-glycolide) copolymer having an inherent viscosity of about 0.16 to about 0.24 dL/g,
(ii) 15 ± 5% of an ester end-capped biodegradable poly(D,L-lactide-co-glycolide) homopolymer having an inherent viscosity of about 0.25 to about 0.35 dL/g, and
(iii) 48 ± 5% of an ester end-capped biodegradable poly(D,L-lactide) homopolymer having an inherent viscosity of about 1.8 to about 2.2 dL/g; or
(c) the biodegradable polymer matrix comprises:
(i) 22 ± 5% of a poly(D,L-lactide) homopolymer having an inherent viscosity of about 0.25 to about 0.35 dL/g, and
(ii) 45 ± 5% of a poly(D,L-lactide) homopolymer having an inherent viscosity of about 1.8 to about 2.2 dL/g;
wherein the inherent viscosity is measured at 0.1% w/v in CHCl₃ at 25° C. with a Ubbelhode size 0c glass capillary viscometer;.

190. The pharmaceutical composition of claim 189, wherein the therapeutic agent is latanoprost, travoprost, bimatoprost, tafluprost, unoprostone isopropyl, or a combination of two or more thereof.

191. The pharmaceutical composition of claim 189, wherein the therapeutic agent is a prostaglandin or a prostaglandin analog.

192. The pharmaceutical composition of claim 189, wherein the therapeutic agent is a prostamide or a prostamide analog.
